(19) United States
(12) Patent Application Publication
(10) Pub. No.: US 2020/0255900 A1
(43) Pub. Date: Aug. 13, 2020

(54) MicroRNA-455-3p as a Peripheral Biomarker for Alzheimer's Disease

(71) Applicant: Texas Tech University System, Lubbock, TX (US)

(72) Inventors: P. Hemachandra Reddy, Lubbock, TX (US); Subodh Kumar, Lubbock, TX (US)

(21) Appl. No.: 16/865,392

(22) Filed: May 3, 2020

Related U.S. Application Data
(63) Continuation-in-part of application No. 16/630,068.
(60) Provisional application No. 62/531,760, filed on Jul. 12, 2017.

(51) Int. Cl. C12Q 1/6883; A61K 31/7105; C12Q 1/686; A61K 9/51

(52) U.S. Cl. CPC C12Q 1/6883; A61K 31/7105; C12Q 1/686; A61K 9/127; C12Q 2600/178; C12Q 2561/113; A61K 9/51

(57) ABSTRACT

The present invention includes a method of protecting neuronal cells from Aβ-induced toxicities in a subject comprising: providing the subject in need of treatment for an Aβ-induced toxicity with an agent that increases the miRNA-445-3p in the subject, wherein an increase in miR-455-3p expression in the neuronal cells enhances neuronal cell survival.

### CROSS-REFERENCE TO RELATED APPLICATIONS

This application is a continuation-in-part application of U.S. patent application Ser. No. 16/630,068 filed Jan. 10, 2020, which is a National Stage of International Application No. of PCT/US2018/041840, filed on Jul. 12, 2018 claiming the priority of U.S. Provisional Application No. 62/531,760, filed on Jul. 12, 2017, the content of each of which is incorporated by reference herein.

### TECHNICAL FIELD OF THE INVENTION

The present invention relates in general to the treatment of Alzheimer's Disease.

### INCORPORATION-BY-REFERENCE OF MATERIALS FILED ON COMPACT DISC

The present application includes a Sequence Listing, which has been submitted in ASCII format via EFS-Web and is hereby incorporated by reference in its entirety. Said ASCII copy, created on Jul. 12, 2018, is named TECH2106WO_SeqList and is 3 kilobytes in size.

### BACKGROUND OF THE INVENTION

Without limiting the scope of the invention, its background is described in connection with biomarkers for Alzheimer's Disease.

One such invention is taught in U.S. Pat. No. 9,188,595, issued to Zhao, et al., and entitled “Alzheimer's disease diagnosis based on mitogen-activated protein kinase phosphorylation.” Briefly, these inventors teach a method of diagnosing Alzheimer's disease in a patient by determining whether the phosphorylation level of an indicator protein in cells of the patient after stimulus with an activator compound is abnormally elevated as compared to a basal phosphorylation level, the indicator protein being e.g. Erk1/2 and the activator compound is bradykinin.

Another invention is taught in United States Patent Publication No. 20140378439, filed by Derso, et al., entitled “MicroRNA Biomarkers Indicative Of Alzheimer's Disease.” These inventors are said to teach a method of diagnosing Alzheimer's Disease in a subject, by determining the level of at least one miRNA in a sample derived from the subject, wherein a change in the level of the at least one miRNA relative to a suitable control is indicative of Alzheimer's Disease in the subject. Methods for monitoring the course of Alzheimer's Disease, methods of treating a subject having Alzheimer's Disease, and kits for diagnosing Alzheimer's Disease are also said to be taught.

Yet another invention is taught in United States Patent Publication No. 20140302068, filed by Khoo, et al., entitled “MicroRNA Biomarkers for Diagnosing Parkinson's Disease”, which is said to teach the identification, development and validation of plasma-based circulating microRNA (miRNAs) biomarkers useful in determining if a subject has Parkinson's disease (PD), is at increased risk of developing PD, or has PD that is progressing or is in remission.

Another such invention is taught in United States Patent Publication No. 20140031245, filed by Khan, et al., and entitled “Alzheimer's Disease-Specific Alterations Of The Erk1/Erk2 Phosphorylation Ratio-Alzheimer's Disease-Specific Molecular Biomarkers (ADSMB)”. Briefly, these applicants are said to teach methods of diagnosing Alzheimer's Disease as well as to methods of confirming the presence or absence of Alzheimer's Disease in a subject. These application methods of identifying a lead compound useful for the treatment of Alzheimer's Disease by contacting non-Alzheimer's cells with an amyloid beta peptide, stimulating the cells with a protein kinase C activator, contacting the cells with a test compound, and determining the value of an Alzheimer's Disease-specific molecular biomarker. The invention is also said to be directed to kits containing reagents for the detection and diagnosis of the presence or absence of Alzheimer's Disease using the Alzheimer's Disease-specific molecular biomarkers disclosed.

Yet another invention is taught in International Patent Publication No. WO2009009457A1, filed by Wang, et al., entitled “Alzheimer's disease-specific micro-RNA microarray and related methods.” These inventors are said to disclose the diagnosis and/or prognosis of Alzheimer's disease in subjects by measuring amounts of one or more micro-RNAs correlated with Alzheimer's disease present in a biological sample, including blood for example, from a subject.

Despite the prior art disclosures, compositions and methods to detect Alzheimer's disease (AD) early, before clinical symptoms develop, are urgently needed to intervene as soon as possible in disease progression. Also needed are early peripheral microRNA (miRNA) biomarkers for AD.

### SUMMARY OF THE INVENTION

In one embodiment, the present invention includes a method of protecting neuronal cells from Aβ-induced toxicities in a subject comprising: providing the subject in need of treatment for an Aβ-induced toxicity with an agent that increases the miRNA-445-3p in the subject, wherein an increase in miR-455-3p expression in the neuronal cells enhances neuronal cell survival. In one aspect, the agent that increases the miRNA-445-3p in the subject is a nucleic acid vector that expresses miRNA-445-3p in neuronal cells of the subject. In another aspect, the agent enhances at least one of: mitochondrial biogenesis, enhances synaptic activity or mitochondrial health. In another aspect, the method comprises providing the subject with a cyclooxygenase inhibitor, a Catecholamine transferase inhibitor, a protein kinases inhibitor, a Neurotransmitter transporter inhibitor, a Renin-angiotensin system inhibitor, a EGFR tyrosine kinase inhibitor, or a HMG-CoA reductase inhibitor. In another aspect, the method comprises identifying a subject in need of treatment for the Aβ-induced toxicity by detecting the presence or the increase in miRNA-445-3p to produce a score that is indicative of a likelihood of developing AD, wherein a higher score relative to a healthy control indicates that the patient is likely to have the prognosis for transitioning to classified AD, wherein the healthy control is derived from a non-AD patient with no clinical evidence of AD. In another aspect, the patient is identified at least 0.1, 0.9, 2.0, 3.5, or greater than 3.5 years prior to reaching clinical disease classification. In another aspect, the method comprises assessing comprises RT-PCR, qRT-PCR, biochip, singleplexed or multiplexed RT-PCR. In another aspect, the method comprises assessing at least one additional biomarker selected from: hsa-miR-3613-3p and hsa-miR-4668-5p, which is up-regulated. In another aspect, the method comprises assessing at least one additional biomarker selected from: hsa-mir-320d-2, hsa-miR-378h, hsa-miR-3921, hsa-miR-6805-5p, hsa-miR-92a-3p, hsa-miR-3613-5p, which is down-regulated. In another aspect, the method comprises obtaining the dataset associated with the sample comprises obtaining the sample and processing the sample to experimentally determine the dataset, or wherein obtaining the dataset associated with the sample comprises receiving the dataset from a third party that has processed the sample to experimentally determine the dataset. In another aspect, the method comprises identifying a relative of the patient at risk for AD by obtaining a score from a dataset associated with a blood, serum, or plasma sample from a relative of the AD patient prior to reaching clinical disease classification. In another aspect, the healthy control is a pre-determined average level derived from a healthy individual with no clinically documented evidence of AD. In another aspect, at least one of: the miR-455-3p has a greater that 20-fold increase in expression in Braak stage V and VI compared to controls; the expression of miR-3613-3p is higher in the brain tissues at Braak stage V when compared to controls; the expression of miR-4668-5p up-regulated in AD brains at Braak stages IV, V, and VI, but less than miR-455-3p or MiR-4674; the expression of mir-6722 was down-regulated in AD serum samples Braak stages I to III, but increased in the AD patients at Braak stage VI, V, and VI; the upregulation of miR-455-3p was significantly higher in postmortem brains from AD patients at Braak stage V having an ApoE (3/4) genotype when compared to controls; or the Braak stage can be differentiated between Stage I to III versus Brask Stage IV to VI by comparing the expression of miR-455-3p, miR-3613-3p, miR-4668-5p, and mir-6722. In another aspect, the agent is an expression vector that expresses miRNA-455-3p. In another aspect, the agent is recombinant miRNA that comprises miRNA-445-3p. In another aspect, the agent is recombinant miRNA that comprises a synthetic miRNA-445-3p miRNA. In another aspect, the agent is a synthetic miRNA-445-3p miRNA in a liposome, vesicle, polymer, or nanoparticles.

In another embodiment, the present invention includes a method for treating at least one of amyloid precursor protein (APP) processing, amyloid beta (Aβ) formation, defective mitochondrial biogenesis/dynamics and synaptic damage in AD progression in a subject suspected of having Alzheimer's disease (AD) comprising: providing the subject in need of treatment for an Aβ-induced toxicity with an agent that increases the miRNA-445-3p in the subject, wherein an increase in miR-455-3p expression in the neuronal cells enhances neuronal cell survival, wherein the amount is sufficient to at least one of correct amyloid precursor protein (APP) processing, prevent amyloid beta (Aβ) formation, decrease defective mitochondrial biogenesis/dynamics or reduce or prevent synaptic damage in AD progression. In another aspect, the agent that increases the expression of miRNA-455-3p in a subject is a nucleic acid vector that expresses miRNA-445-3p in neuronal cells of the subject, wherein an increase in miR-455-3p expression in the neuronal cells enhances neuronal cell survival, wherein the amount is sufficient to at least one of correct amyloid precursor protein (APP) processing, prevent amyloid beta (Aβ) formation, decrease defective mitochondrial biogenesis/dynamics or reduce or prevent synaptic damage in AD progression. In another aspect, the expression of miRNA-445-3p in neuronal cells reduces a level of mutant APP, Aβ(1-40) and Aβ(1-42), and C99 by miR-455-3p. In another aspect, the method comprises administering a cyclooxygenase inhibitor, a Catecholamine transferase inhibitor, a protein kinases inhibitor, a Neurotransmitter transporter inhibitor, a Renin-angiotensin system inhibitor, a EGFR tyrosine kinase inhibitor, or a HMG-CoA reductase inhibitor. In another aspect, the method comprises assessing at least one additional biomarker selected from: hsa-miR-3613-3p and hsa-miR-4668-5p, which is up-regulated. In another aspect, the method comprises assessing at least one additional biomarker selected from: hsa-mir-320d-2, hsa-miR-378h, hsa-miR-3921, hsa-miR-6805-5p, hsa-miR-92a-3p, hsa-miR-3613-5p, which is down-regulated. In another aspect, the patient is identified at least 0.1, 0.9, 2.0, 3.5, or greater than 3.5 years prior to reaching clinical disease classification. In another aspect, the step of assessing comprises RT-PCR, qRT-PCR, biochip, singleplexed or multiplexed RT-PCR. In another aspect, the agent is an expression vector that expresses miRNA-455-3p. In another aspect, the agent is recombinant miRNA that comprises miRNA-445-3p. In another aspect, the agent is recombinant miRNA that comprises a synthetic miRNA-445-3p miRNA. In another aspect, the agent is a synthetic miRNA-445-3p miRNA in a liposome, vesicle, polymer, or nanoparticles.

In another embodiment, the present invention includes a method for treating a subject with Alzheimer's Disease (AD) comprising: obtaining a blood, serum, cerebrospinal fluid, or plasma sample from the AD patient; assessing the dataset for a presence or an increase in an amount of miRNA-445-3p; and if the subject has a decrease in miRNA-445-3p expression or is in need of an increase in the expression of miRNA-445-3p, providing the subject in need of treatment for an Aβ-induced toxicity an agent that increases the miRNA-445-3p in the subject, wherein an increase in miR-455-3p expression in the neuronal cells enhances neuronal cell survival. In one aspect, the agent that increases the miRNA-445-3p in the subject is a nucleic acid vector that expresses miRNA-445-3p in neuronal cells of the subject. In another aspect, the method comprises administering a cyclooxygenase inhibitor, a Catecholamine transferase inhibitor, a protein kinases inhibitor, a Neurotransmitter transporter inhibitor, a Renin-angiotensin system inhibitor, a EGFR tyrosine kinase inhibitor, or a HMG-CoA reductase inhibitor. In another aspect, the step of assessing comprises RT-PCR, qRT-PCR, biochip, singleplexed or multiplexed RT-PCR. In another aspect, the method comprises assessing at least one additional biomarker selected from: hsa-miR-3613-3p and hsa-miR-4668-5p, which is up-regulated. In another aspect, the method further comprises assessing at least one additional biomarker selected from: hsa-mir-320d-2, hsa-miR-378h, hsa-miR-3921, hsa-miR-6805-5p, hsa-miR-92a-3p, hsa-miR-3613-5p, which is down-regulated. In another aspect, the dataset associated with the sample comprises obtaining the sample and processing the sample to experimentally determine the dataset, or wherein obtaining the dataset associated with the sample comprises receiving the dataset from a third party that has processed the sample to experimentally determine the dataset. In another aspect, the method further comprises identifying a relative of the patient at risk for AD by obtaining a score from a dataset associated with a blood, serum, or plasma sample from a relative of the AD patient prior to reaching clinical disease classification. In another aspect, the healthy control is a pre-determined average level derived from a healthy individual with no clinically documented evidence of AD. In another aspect, the agent is an expression vector that expresses miRNA-455-3p. In another aspect, the agent is recombinant miRNA that comprises miRNA-445-3p. In another aspect, the agent is recombinant miRNA that comprises a synthetic miRNA-445-3p miRNA. In another aspect, the agent is a synthetic miRNA-445-3p miRNA in a liposome, vesicle, polymer, or nanoparticles.

In another embodiment, the present invention includes a composition comprising an amount of an agent that upregulates the expression of miRNA-445-3p. In one aspect, the agent is a recombinant miRNA-455-3p nucleic acid in a nucleic acid vector that expresses miRNA-445-3p in neuronal cells of a subject. In another aspect, the agent is an expression vector that expresses miRNA-455-3p. In another aspect, the agent is recombinant miRNA that comprises miRNA-445-3p. In another aspect, the agent is recombinant miRNA that comprises a synthetic miRNA-445-3p miRNA. In another aspect, the agent is a synthetic miRNA-445-3p miRNA in a liposome, vesicle, polymer, or nanoparticles.

In another embodiment, the present invention includes a method of treating a subject in need of therapy for Alzheimer's Disease comprising: identifying a subject suspected of having Alzheimer's Disease; and providing the subject with an effective amount of a recombinant miRNA-445-3p expression vector sufficient to upregulate the expression of miRNA-445-3p in the subject in need of therapy for Alzheimer's Disease.

### DESCRIPTION OF THE DRAWINGS

For a more complete understanding of the features and advantages of the present invention, reference is now made to the detailed description of the invention along with the accompanying figures and in which:

FIG. 1 is a heat map showing hierarchical clustering of miRNAs in AD patients and healthy controls. Left side showed the Transcript cluster ID of differentially expressed 7 miRNAs. Red and green color indicated high and low expression intensities.

FIGS. 2A to 2E show qRT-PCR validation of serum samples. Expression of (FIG. 2A) miR-455-3p, (FIG. 2B) miR-4668-5p, (FIG. 2C) miR-4674, (FIG. 2D) miR-3613-3p and (FIG. 2E) mir-6722 in healthy controls, MCI subjects and AD patients' serum samples. Fold change was calculated by 2−Δct method. Significant difference among groups were calculated by one-way ANOVA with P<0.05 is considered statistically significant.

FIGS. 3A to 3E show qRT-PCR analysis of AD postmortem brains. Specificity and Expression of (FIG. 3A) miR-455-3p, (FIG. 3B) miR-3613-3p, (FIG. 3C) miR-4674, (FIG. 3D) miR-4668-5p and (FIG. 3E) mir-6722 in AD postmortem brain tissues at Braak Stages (BS) IV, V and VI. Fold change was calculated by 2-ΔΔct method. Significant difference among groups were calculated by one-way ANOVA with P<0.05 is considered statistically significant.

FIGS. 4A and 4B show the specificity and sensitivity analysis. ROC curve analysis of miR-455-3p in (FIG. 4A) serum samples from AD patients and (FIG. 4B) AD postmortem brain tissue samples.

FIGS. 5A to 5C show qRT-PCR analysis of miR-455-3p in mice model. mmu-miR-455-3p expression in (FIG. 5A) Cerebral cortex, (FIG. 5B) Cerebellum and (FIG. 5C) Serum of APP-mice compared to wild type mice. Fold change was calculated by 2-ΔΔct method. Significant difference among groups were calculated by paired t-test with two-tailed P<0.05 is considered significant.

FIGS. 6A and 6B show MiR-455-3p expression in cell lines. qRT-PCR analysis of hsa-miR-455-3p expression in (FIG. 6A) SHSY-5Y cells, and (FIG. 6B) mmu-miR-455-3p expression in N2a cells. Fold change was calculated by 2−ΔΔct method. Significant difference among groups were calculated by paired t-test with two-tailed P<0.05 is considered significant.

FIG. 7 shows a KEGG pathway analysis of miR-455-3p. MiR-455-3p regulated pathways and target genes were identified using the sources microT-CDS and TarBase to classify KEGG pathway and GO category pathway with P<0.05. MiR-455-3p targeted pathway genes identified through literature survey that were implicated in AD pathogenesis.

FIG. 8 shows MicroRNA 455-3p as a peripheral biomarker for Alzheimer's disease.

FIG. 9 shows the details of a microRNA 455-3p study.

FIGS. 10A to 10C show that expression of hsa-miR-455-3p in AD patients. (FIG. 10A) miR-455-3p expression in the postmortem brains of healthy controls (n=15) and AD patients' (n=32) was quantified by real-time RT-PCR. Data are presented as “-delta CT” values using box and whiskers plots. Significant difference between groups were calculated by unpaired t-test with P<0.05 is considered statistically significant. (FIG. 10B) Expression of hsa-miR-455-3p in human fibroblast cells from healthy controls (n=8), Familial AD cases (n=4) and sporadic AD patients' (n=6). Significant difference between groups were calculated by one-way ANOVA with P<0.05 is considered statistically significant. (FIG. 10C) Expression of hsa-miR-455-3p in human B-lymphocytes cells from healthy controls (n=10), Familial AD cases (n=6) and sporadic AD patients' (n=6). Significant difference between groups were calculated by one-way ANOVA with P<0.05 is considered statistically significant.

FIGS. 11A to 11C show that ROC curve analysis of hsa-miR-455-3p in (FIG. 11A) AD postmortem brains, (FIG. 11B) AD fibroblast cell lines, and in (FIG. 11C) B-lymphocytes cells from AD patients. The curve was plotted based on the 1CT value of miR-455-3p in AD patients and control samples. Area under the ROC curve (AUROC) was calculated along with the sensitivity and specificity values. P<0.05 is considered statistically significant.

FIGS. 12A to 12D show scattered plot diagrams showing the Pearson correlation coefficient (r) values of miR-455-3p expression with (FIG. 12A) AD postmortem brains autolysis time (FIG. 12B) Age of AD postmortem brains (FIG. 12C) Age of AD fibroblast cells and (FIG. 12D) Age of AD B-lymphocytes.

FIGS. 13A to 13H—MiR-455-3p interaction with the APP gene and its effects on neuroblastoma cells survival (FIG. 13A) The two putative binding sites of miR-455-3p at the 3′-UTR regions of a wild-type APP gene in humans and mice. Region A (522-528) had 7 nucleotide binding sites, and Region B (3139-3145) had 6 nucleotide binding sites with the seed sequence of miR-455-3p in both the human and mouse wild-type APP gene (shown in green). (FIG. 13B) Luciferase reporter assay for miR-455-3p binding with the APP gene. Normalized luciferase activity (Firefly/Renilla) in neuroblastoma cells co-transfected with the APP 3′UTR clone (HmiT009578-MT06) and miR-455-3p mimics. Firefly/Renilla luciferase activity of the APP 3′UTR clone was significantly decrease by the miR-455-3p. (FIG. 13C) Representative images of neuroblastoma cells morphology after 24 hr of miR-455-3p mimics and inhibitor transfection (10× magnification). Quantitative measurement of miR-455-3p (FIG. 13D), and APP fold-change (FIG. 13E), in cells at 24 hr post mimics and inhibitor transfection. (13F) Representative images of Annexin V and PI staining of cells at 24 hr post-transfection of miR-455-3p mimics and inhibitor. White arrow represents the viable (green) and dead (red) cells. Percentage (%) of apoptotic cells (FIG. 13G), and viable cells populations (FIG. 13H), after 24 hr of miR-455-3p mimics and inhibitor transfection. (*P<0.05) (**P<0.01) (***P<0.001)

FIGS. 14A to 14J—Regulation of mutant APP cDNA expression by miR-455-3p. (FIG. 14A) Representative images for transfection of the miR-455-3p expression vector (GFP), the miR-control (GFP) vector, and the mutant APP cDNA in neuroblastoma cells (10× magnification). Bright field images and green fluorescence detected in the same field at 24 hr post-transfection (10× magnification). (FIG. 14B) Representative images of Annexin V and Propidium Iodide (PI) stained neuroblastoma cells transfected with the miR-control vector, the miR-455-3p vector, and the mutant APP cDNA. The white arrow indicates the populations of viable (green) cells and dead (red) cells. (FIG. 14C) Percentage (%) of an apoptotic cell population after 24 hr of the miR-455-3p vector, the miR-control vector, and the mutant APP cDNA transfecting cells. (FIG. 14D) Percentage (%) of a viable cell population after 24 hr of the miR-control vector, the miR-455-3p vector and the mutant APP cDNA transfecting cells. (FIG. 14E) Quantitative measurement of miR-455-3p fold change expression after 24 hr of the miR-455-3p vector and the mutant APP cDNA transfecting cells. (FIG. 14F) Quantitative measurement of APP mRNA folds change expression after 24 hr of the miR-455-3p vector and the mutant APP cDNA transfecting cells. (FIG. 14G) Western blot for APP (6E10), the C-terminal fragment of APP (C99) and B-actin proteins in cells transfected with the miR-control vector, the miR-455-3p vector, mutant APP cDNA and co-transfected cells. Quantitative measurement of (FIG. 14H) APP (6E10) and C-terminal fragments of APP C99 (FIG. 14I), and C83 (FIG. 14J) proteins levels by densitometry in the mutant APP cDNA and the miR-455-3p vector transfecting cells. (*P<0.05) (**P<0.01) (***P<0.001).

FIGS. 15A to 15C—MiR-455-3p reduces APP and amyloid proteins. (FIG. 15A) Representative immunostaining images of neuroblastoma cells transfected with the miR-control vector, the miR-455-3p vector, the mutant APP cDNA, the mutant APP and miR-455-3p co-transfected. Cells were stain with the APP (6E10) antibody producing red fluorescence (20× magnification). Green fluorescence showed the miR-control and miR-455-3p vector expression and the nucleus of the cell stained with DAPI (blue). Fluorescence intensity (red) of the mutant APP was reduce in mutant APP and miR-455-3p co-transfected cells. (FIG. 15B) Quantitative measurement of fluorescence intensity of the APP (6E10) protein in mutant APP, miR-455-3p, and miR-control vector transfected cells. (FIG. 15C) ELISA analysis for amyloid ₍₁₋₄₀₎ and ₍₁₋₄₂₎ levels in neuroblastoma cells. Quantitative detection of the (i) human amyloid-β₍₁₋₄₀₎ and (ii) amyloid-β₍₁₋₄₂₎ peptide level in mutant APP cDNA and miR-455-3p vector transfected cells. (*P<0.05)

FIGS. 16A to 16H—Immunoblotting for mitochondrial biogenesis and synaptic genes. (FIG. 16A) Representative western blot images for PGC1α, NRF1, NRF2, TFAM, and beta-actin proteins levels in 1) Neuroblastoma cells transfected with the miR-control vector, 2) Neuroblastoma cells transfected with the miR-455-3p expression vector, 3) Neuroblastoma cells transfected with the mutant APP cDNA, 4) Neuroblastoma cells co-transfected with the miR-455-3p and mutant APP cDNA, and 5) Neuroblastoma cells co-transfected with the mutant APP and miR-control vector. Quantitative measurement of the levels of (FIG. 16B) the PGC1α protein (FIG. 16C) NRF1, (FIG. 16D) NRF2, and (FIG. 16E) TFAM using densitometry in 1) Cells transfected with the miR-control vector, 2) Cells transfected with the miR-455-3p expression vector, 3) Cells transfected with mutant APP cDNA, 4) Cells co-transfected with the miR-455-3p and mutant APP cDNA, and 5) Cells co-transfected with the mutant APP and miR-control vector. (FIG. 16F) Representative western blot images for synaptophysin, PSD95, and beta-actin proteins levels in 1) Cells transfected with the miR-control vector, 2) Cells transfected with the miR-455-3p expression vector, 3) Cells transfected with the mutant APP cDNA, 4) Cells co-transfected with the miR-455-3p and mutant APP cDNA, and 5) Cells co-transfected with the mutant APP and miR-control vector. Quantitative measurement of the levels of (G) the synaptophysin protein and (FIG. 16H) PSD95 using densitometry in same groups of cells. (*P<0.05) (**P<0.01)

FIGS. 17A to 17I—Immunoblotting for mitochondrial dynamic genes. (FIG. 17A) Representative western blot images for DRP1, FIS1, OPA1, Mfn1, Mfn2 and beta-actin proteins levels in: 1) Neuroblastoma cells transfected with the miR-control vector, 2) Neuroblastoma cells transfected with the miR-455-3p expression vector, 3) Neuroblastoma cells transfected with the mutant APP cDNA, 4) Neuroblastoma cells co-transfected with the miR-455-3p and mutant APP cDNA, and 5) Neuroblastoma cells co-transfected with the mutant APP and miR-control vector. Quantitative measurement of the levels of (FIG. 17B) the DRP1, (FIG. 17C) FIS1, (FIG. 17D) OPA1, (FIG. 17E) Mfn1 and (FIG. 17F) Mfn2 proteins using densitometry in the same groups of cells. (FIG. 17G) Representative TEM images of Neuroblastoma cells transfected with the miR-control vector, the miR-455-3p vector, mutant APP cDNA, co-transfected with mutant APP and miR-455-3p, and co-transfected with mutant APP and miR-control, showing mitochondrial organization (600 nm magnification). (FIG. 17H) Quantification of the number of mitochondria in the Neuroblastoma cells transfected with miR-control vector, the miR-455-3p vector, mutant APP cDNA, co-transfected with mutant APP and miR-455-3p, and co-transfected with mutant APP and miR-control. (FIG. 17I) Quantification of the size of mitochondria (μm) in the neuroblastoma cells transfected with miR-control vector, the miR-455-3p vector, mutant APP cDNA, co-transfected with mutant APP and miR-455-3p, and co-transfected with mutant APP and miR-control. (*P<0.05) (**P<0.01).

FIG. 18 shows a proposed mechanism of action mt miRNAs on the mitochondrial function, ATP production, and synaptic activity.

FIG. 19 is a working model of synaptic miRNAs: miRNAs localized in dendrites and axon terminals could modulate the expression of local mRNAs and proteins.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Alzheimer's disease (AD) is progressive neurological disorder affecting aged humans (1). The loss of memory, thinking skills, reasoning abilities, and changes in personality and in behavior are the main characteristics of AD (2). Currently, over 46.8 million people worldwide, including 5.4 million Americans, live with AD-related dementia, and this number is estimated to increase to 131.5 million by 2050 (2). The major pathological hallmarks of AD are the formation of extracellular amyloid plaques and intracellular neurofibrillary tangles (NFTs) in brains of patients with AD. The amyloid plaques accumulate due to the overproduction of the amyloid β peptide (Aβ). This overproduction is due to endoproteolysis of the parental amyloid precursor protein (APP), which is cleaved by the enzyme complexes α-, β-, and γ-secretases (3). Increased production and reduced clearance of Aβ in the brain, may lead to a cascade of events in disease process, including synaptic damage, hyperphosphorylated tau (p-tau), mitochondrial structural and functional changes, inflammatory responses, hormonal imbalance, cell cycle changes, and neuronal loss (4-6).

Currently, to diagnose AD, several biochemical tests are used to detect Aβ and p-tau proteins in the cerebrospinal fluid (CSF) of AD patients. This fluid then undergoes biochemical and molecular tests, to determine the levels of biomarkers of AD. In the CSF of patients diagnosed with AD, the concentration of Aβ(1-42) has been found to be 40-50% lower than concentration levels in individuals who do not have AD (6). Such lower levels of Aβ(1-42) in patients have been detected at later stages of AD progression, but they have not been detected in patients in early stages of disease progression. The use of CSF analysis to determine levels of Aβ(1-42) is considered a safe procedure, but often times, the patients complain about post-examination headaches (7). CSF examination requires highly skilled persons puncturing the lumbar to remove spinal fluid. Additional testing to diagnose AD uses highly sophisticated neuroimaging techniques, such as positron emission tomography and structural magnetic resonance imaging and scanning (8).

Neurodegenerative diseases such as Alzheimer's (AD) and Parkinson's are very debilitating diseases. While the cause of Alzheimer's disease is not known, it is generally accepted that a build up of beta-amyloid (Aβ) plaques in the brain cause the interruption of signaling pathways. This build up of beta-amyloid (Aβ) plaques causes memory problems, language difficulties, mood swings, and other debilitating symptoms. While there is no cure for Alzheimer's disease, early detection of the disease leads to a better prognosis.

### Example 1

The primary subject matter of the disclosed invention includes the use of MicroRNAs (miRNAs) found in the blood serum to detect the presence or predict future presence of AD. MicroRNAs are small segments of RNA that are a recent discovery and are currently being heavily researched. Generally, miRNAs are utilized by the body to regulate certain functions. It has been proposed that miRNAs might be useful as biomarkers for detecting disease where there is no other viable method of detection.

In the disclosed technology, miRNA-445-3p has been identified as a biomarker for detecting Alzheimer's disease. The results disclosed herein confirm that when a higher level of miRNA-455-3p is found in a patient's serum, this indicates the presence of Alzheimer's with both high sensitivity and specificity. The miRNA levels of known Alzheimer's patients against non-disease control patients to determine this correlation.

The invention uses miRNA-455-3p as a biomarker for detecting Alzheimer's at its earliest stage. The only current and definitive test for Alzheimer's is postmortem analysis of the brain. With the method disclosed herein, a single blood sample would be capable of measuring the circulating levels of miRNA-455-3p. These measurements would allow physicians to more confidently diagnose and detect Alzheimer's in the earliest stages of the disease.

Given these problems with diagnostic tests for AD, in the last decade researchers have focused on developing non-invasive diagnostic tests capable of detecting nucleic acids, particularly microRNA (miRNAs), known to regulate in patients with AD. These miRNAs are small nucleotide molecules (˜22-25 measurement unit) that expressed in humans, plants, fungi, bacteria, and some viruses (9). In neurodegenerative diseases like AD, miRNAs have been found to be deregulated in the blood, plasma, serum, CSF, extracellular fluid, and brain tissues of AD patients (10,11).

In humans, miRNAs are believed to be involved in all developmental and pathological processes by regulating gene expression. They achieve this regulation by targeting 3′ UTR and binding RNA sequences at 3′UTR in a sequence-specific manner (12). Some miRNAs are tissue-specific and are localized at certain cellular niches, while others are expressed in all tissues and organs of human body. MiRNAs synthesized in the cells and usually modulate mRNA activity of host cells while in several circumstances, miRNAs released from cells are involved in regulating signals for cells-to-cell communication, known as extracellular miRNAs (13). Extracellular miRNAs are secreted from cells via encapsulated exosomes and micro-particles, or they are released with several lipoprotein complexes, such as high-density lipoproteins (HDL), low-density lipoproteins (LDL), and argonaute 2 proteins (13). These extracellular circulatory miRNAs are very stable in blood components. In pathological conditions, such as in persons with AD, concentrations of particular miRNAs are altered (11). However, the inventors still do not have complete understanding of how expressions(s) of miRNAs progress in non-demented elderly individuals to mild cognitive impairment (MCI), and MCI to AD.

Several recent miRNA studies using CSF, serum, plasma and whole blood revealed that circulatory miRNAs as peripheral biomarkers in AD (6,14-22). However, these studies provided information about miRNAs with little or no consensus in all studies. Further, validation of differentially expressed miRNAs using AD postmortem brains is not well done in these studies.

Therefore, a more detailed study on circulatory miRNAs in AD patients and MCI subjects with thorough validation is urgently needed, in order to determine early detectable peripheral biomarkers in AD. In the present study, the inventors screened AD patients, MCI subjects, and healthy controls for circulatory miRNAs in serum samples using an Affymetrix microarray and qRT-PCR validation assay. Further, differentially expressed miRNAs were validated using AD postmortem brains, APP transgenic mice and AD cell lines.

Levels of serum miRNAs. Total RNA was extracted from 40 serum samples, for microarray analysis, the concentration of miRNAs (10-40 nucleotides) and small RNAs (0-257 nucleotides), and the ratio of miRNAs to small RNAs in each sample were analyzed. The RNA levels were calculated by an Agilent 2100 Bioanalyzer (Agilent Technologies). The average concentration of miRNAs in AD patients was 89.1 pg/μ1, in MCI subjects, 132.7 pg/μl and in controls was 119.3 pg/μ1. The average concentration of small RNAs was 186 pg/μl in AD patients, 248.5 pg/μl in MCI subjects and 240.2 pg/μl in controls. Similarly, the ratios of average miRNAs to small RNAs in the samples were 49.1%, 54.9% and 49.8% in AD patients, MCI subjects and in controls respectively. These results indicated that miRNA output was greater in the MCI subjects.

Primary screening of serum samples to detect miRNAs. AD patients (n=10), MCI subjects (n=16), and controls (n=14) were analyzed for their miRNA microarray expression using the Affymetrix GeneChip miRNA Array, v. 4.0. A total of 6631 genes were detected in all of the serum samples. Of these 6631 genes, 2578 were mature miRNAs that were listed in the miRbase database, and 2025 were the stem-loop precursor miRNAs (pre-miRNAs). The remaining genes belonged to different classes of small RNAs, such as snoRNA (1491), CDBox (319), HAcaBox (155), scaRna (31), and 5.8s rRNA (10). Of the remaining genes, 22 were spike-in control RNAs that were added externally during the array experiment. Differential miRNA expression in each miRNA was analyzed, on the fold-change intensity of each miRNA (−2 to +2) and each ANOVA P-value (<0.05).

AD patients and healthy controls. Microarray analysis was performed on the samples from the AD patients (n=10) and the controls (n=14) (FIG. 1). The miRNA bi-weight average (log 2) intensity showed significant (P<0.05) deregulation of 7 miRNAs in AD patients compared to controls (Table 1). The miRNA sequences, hsa-miR-455-3p, hsa-miR-3613-3p, hsa-miR-4668-5p, hsa-miR-5001-5p, hsa-miR-4674, and hsa-miR-4741 were up-regulated, while hsa-miR-122-5p was down-regulated. The top miRNA candidate was hsa-miR-455-3p, which showed a remarkably 11.3-fold higher expression in AD patients compared to controls. Other miRNAs were, hsa-miR-3613-3p (3.67-fold), hsa-miR-4668-5p (3.38-fold), and hsa-miR-4674 (5.62-fold) also exhibited the higher levels of fold expression in AD patients. These results identified new miRNA candidates that were not previously identified in AD.

TABLE 1
MiRNAs: log2 intensity and fold change in AD patients and controls

| | | AD | | Control | | Fold | | | |
|---|---|---|---|---|---|---|---|---|---|
| | | Bi-weight | | Bi-weight | | Change | | | |
| | | Average | AD | Average | Control | (linear) | | | |
| Transcript | miRNA | Signal | Standard | Signal | Standard | (AD vs. | ANOVA | FDR | Chromo- |
| Cluster ID | name | (log2) | Deviation | (log2) | Deviation | Control) | p-value | p-value | some |
| 20504187 | hsa- | 6.03 | 1.05 | 2.53 | 1.06 | 11.3 | 0.000003 | 0.007 | chr9 |
| | miR- | | | | | | | | |
| | 455-3p | | | | | | | | |
| 20517821 | hsa- | 3 | 1.32 | 1.13 | 0.16 | 3.67 | 0.000014 | 0.012 | chr13 |
| | miR- | | | | | | | | |
| | 3613-3p | | | | | | | | |
| 20519463 | hsa- | 3 | 1.5 | 1.25 | 0.42 | 3.38 | 0.000576 | 0.079 | chr9 |
| | miR- | | | | | | | | |
| | 4668-5p | | | | | | | | |
| 20500726 | hsa- | 2.31 | 1.4 | 4.26 | 1.24 | −3.85 | 0.004833 | 0.198 | chr18 |
| | miR- | | | | | | | | |
| | 122-5p | | | | | | | | |
| 20520198 | hsa- | 3.73 | 0.7 | 2.49 | 0.77 | 2.37 | 0.011848 | 0.263 | chr2 |
| | miR- | | | | | | | | |
| | 5001-5p | | | | | | | | |
| 20519474 | hsa- | 4.48 | 1.37 | 1.99 | 1.05 | 5.62 | 0.013827 | 0.275 | chr9 |
| | miR- | | | | | | | | |
| | 4674 | | | | | | | | |
| 20519592 | hsa- | 2.31 | 0.61 | 1.26 | 0.42 | 2.07 | 0.013923 | 0.276 | chr18 |
| | miR- | | | | | | | | |
| | 4741 | | | | | | | | |

AD patients and MCI subjects. To compare the intermediate states of disease progression, microarray data were analyzed from the AD patients (n=10) and MCI subjects (n=16). Heat map data and hierarchical clustering showed the differential expressions of 8 miRNA candidates. Based on the bi-weight average (log 2) intensity and linear fold-change values, the miRNAs hsa-miR-3613-3p and hsa-miR-4668-5p were significantly up-regulated (ANOVA, P<0.05) and hsa-mir-320d-2, hsa-miR-378h, hsa-miR-3921, hsa-miR-6805-5p, hsa-miR-92a-3p, hsa-miR-3613-5p were down-regulated in the serum samples of AD patients compared to MCI subjects.

MCI subjects and healthy controls. Microarray data were compared between MCI subjects (n=16) and controls (n=14). Hierarchical clustering showed a wide range of miRNA signatures that were deregulated. Interestingly, 50 miRNAs were identified, and all of them were significantly up-regulated in MCI subjects. Surprisingly, miR-4674 (5.24-fold) and miR-455-3p (5.18-fold) showed maximum upregulation in MCI subjects, compared to controls. These results suggested that greater number of miRNA deregulation were observed in the initial phases of disease progression.

AD patients, MCI subjects, and healthy controls. To detect disease progression through the differential expression of miRNAs, the inventors compared the differentially expressed miRNAs in the serum samples of AD patients and MCI subjects (n=10 and n=16, respectively) and controls (n=14) at the same time point in disease progression. The miRNAs in each group of serum samples were analyzed in terms of: bi-weight average (log 2) intensity, a fold change of less than −2 or more than 2, and an ANOVA/FDR, P<0.05. Results indicated that a total of 68 miRNAs (32 mature and 36 precursor) were deregulated among three groups of serum samples. Since, present study aimed to identify promising biomarkers for AD progression, the inventors focused on miRNAs, and those expressions are either gradually increased or decreased among three groups. Of the 32 mature miRNAs that were identified, 7 were gradually upregulated: hsa-miR-455-3p, hsa-miR-3613-3p, hsa-miR-4674, hsa-miR-4668-5p, hsa-miR-4317, hsa-miR-3124-3p, and hsa-miR-6856-3p, while one, hsa-miR-1972 was down-regulated in AD and MCI subjects compared to controls. Further, the remaining pre-miRNAs (hsa-mir-124-1, hsa-mir-4417, hsa-mir-1908, hsa-mir-3912, hsa-mir-4325, and hsa-4776-2) showed gradual upregulation while 4 of the pre-miRNAs (hsa-mir-6722, hsa-mir-412, hsa-mir-3153, and hsa-mir-4430) showed gradual downregulation.

Among the 68 miRNAs that the inventors studied, the most significantly upregulated (ANOVA/FDR, P<0.05) were 4 miRNAs miR-455-3p, miR-3613-3p, miR-4674, and miR-4668-5p and the one down-regulated miRNA mir-6722. These miRNAs were selected for secondary screening and validation analysis since their expression varied more in the 3 groups of serum samples (Table 2). The miR-455-3p log 2 intensity showed a 2.53-fold increase in the controls, a 4.9-fold increase in MCI subjects, and a 6.03-fold increase in AD patients. Similarly, the expression of miR-4674 also increased from 1.99-fold in controls, to a 4.38-fold increase in MCI subjects, and to a 4.48-fold increase in AD patients.

TABLE 2
miRNAs: log2 intensity and fold change in AD patients, MCI subjects, and controls

| | | AD Bi- | MCI Bi- | Controls | | | | | | Fold | Fold | Fold |
|---|---|---|---|---|---|---|---|---|---|---|---|---|
| | | weight | weight | Bi-weight | | | | ANOVA | FDR | Change | Change | Change |
| | Tran- | Average | Average | Average | | | | p-value | p-value | (linear) | (linear) | (linear) |
| Transcript | script | Signal | Signal | Signal | AD | MCI | Controls | (All | (All | (AD vs. | (AD vs. | (MCI vs. |
| Cluster ID | ID | (log2) | (log2) | (log2) | SD | SD | SD | Conditions) | Conditions) | MCI) | Controls) | AD) |
| 20504187 | hsa- | 6.03 | 4.9 | 2.53 | 1.05 | 1.29 | 1.06 | 0.000002 | 0.001994 | 2.18 | 11.3 | −2.18 |
| | miR- | | | | | | | | | | | |
| | 455- | | | | | | | | | | | |
| | 3p | | | | | | | | | | | |
| 20537464 | hsa- | 5.72 | 6.27 | 6.72 | 0.45 | 0.48 | 0.4 | 0.000016 | 0.007169 | −1.46 | −1.99 | 1.46 |
| | mir- | | | | | | | | | | | |
| | 6722 | | | | | | | | | | | |
| 20519474 | hsa- | 4.48 | 4.38 | 1.99 | 1.37 | 1.26 | 1.05 | 0.000208 | 0.024285 | 1.07 | 5.62 | −1.07 |
| | miR- | | | | | | | | | | | |
| | 4674 | | | | | | | | | | | |
| 20517821 | hsa- | 3 | 1.25 | 1.13 | 1.32 | 0.64 | 0.16 | 9.42E−07 | 0.001562 | 3.37 | 3.67 | −3.37 |
| | miR- | | | | | | | | | | | |
| | 3613- | | | | | | | | | | | |
| | 3p | | | | | | | | | | | |
| 20519463 | hsa- | 3 | 1.67 | 1.25 | 1.5 | 0.91 | 0.42 | 0.000711 | 0.045154 | 2.52 | 3.38 | −2.52 |
| | miR- | | | | | | | | | | | |
| | 4668- | | | | | | | | | | | |
| | 5p | | | | | | | | | | | |

The miR-4668-5p showed a gradual upregulation of up to 3-fold in AD patients, compared to 1.25-fold increase in controls, and a 1.67-fold increase in MCI subjects. The miR-3613-3p expression also gradually increased when the miRNAs were analyzed and compared in controls, MCI, and AD patients (1.13-fold, 1.25-fold, and 3.0-fold increases, respectively). The expression of mir-6722 gradually decreased in MCI subjects (6.27-fold) and AD patients (5.72-fold) compared to mir-6722 expression (6.72-fold) in controls. Thus, the circulatory serum miRNAs showed aberrant expression in the healthy controls and diseased states (AD and MCI). Also noteworthy was that the level of expression in these molecules consistently either increased or decreased with disease progression. Hence, such miRNAs are capable of discriminating between healthy persons and persons with AD or MCI. Such miRNAs could also be used to monitor disease progression in AD patients.

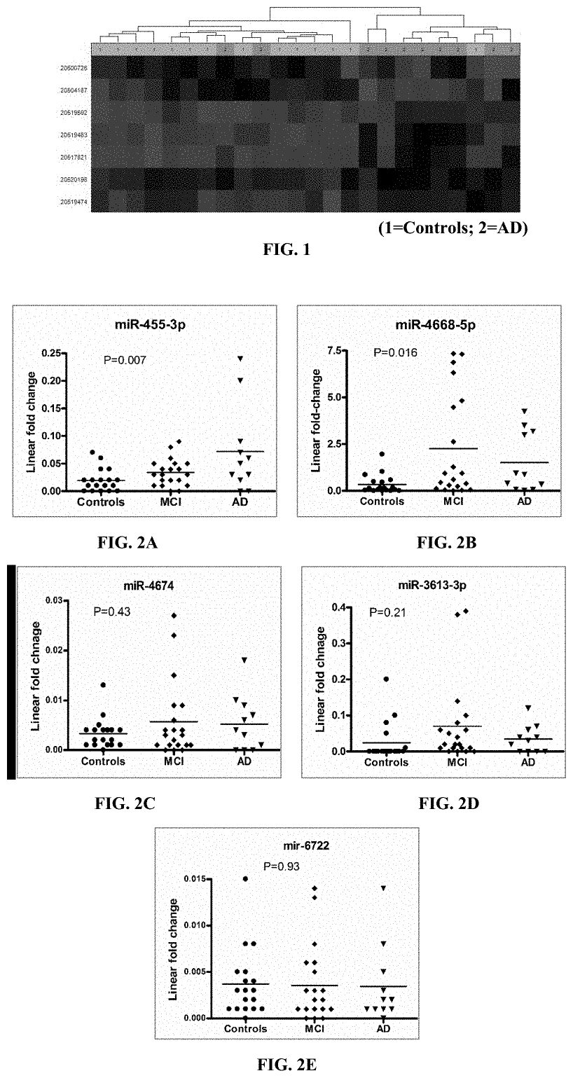

Secondary screening and validation of miRNAs in serum samples. Selected miRNAs (miR-455-3p, miR-3613-3p, miR-4674, miR-4668-5p and mir-6722) were further validated for their expression using qRT-PCR assays. The expression of miR-455-3p was quantified in serum of controls (n=18), MCI subjects (n=20), and AD patients (n=11). Interestingly, fold-change (mean±SD) analysis indicated a gradual upregulation of miR-455-3p in AD patients (0.071±0.078-fold) (P=0.007) compared to the fold-change in MCI subjects (0.034±0.024-fold) and in controls (0.019±0.020-fold) (FIG. 2A). Similarly, the expression of miR-4668-5p was also significantly (P=0.016), upregulated in MCI subjects (2.25±2.78-fold) compared to controls (0.340±0.50-fold) (FIG. 2B). However, miR-4668-5p expression did not show significant elevation in the AD patient's serum (1.50±1.61-fold). In similar way, expressions of miR-4674 and miR-3613-3p also increased in the MCI and AD patients, though it was not significant (FIGS. 2C and 2D).

The expression of precursor miRNA (mir-6722) was also quantified by qRT-PCR, with results showing a gradual down regulation in MCI and AD patients compared to controls, but not significantly (FIG. 2E). A microarray-based panel of 5 miRNAs was found to concur with qRT-PCR validation in controls, MCI and AD serum samples. However, a statistical analysis revealed that miR-455-3p and miR-4668-4p were significantly upregulated in persons with AD or MCI from healthy controls.

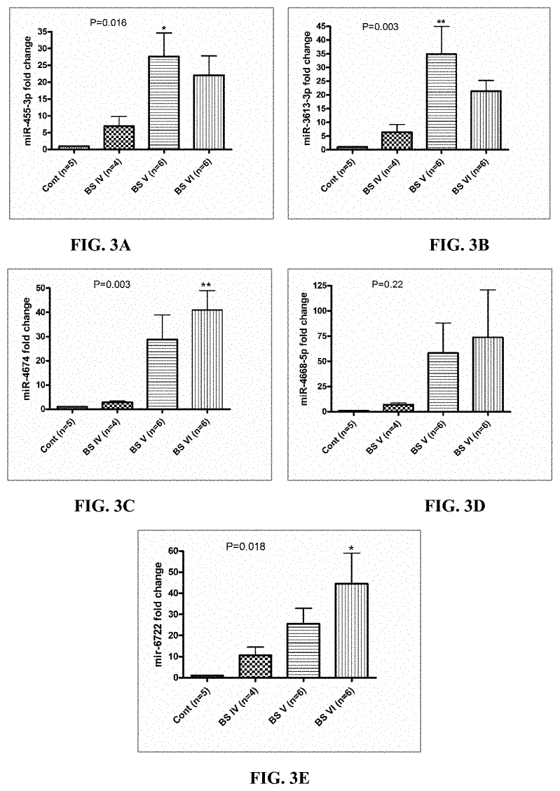

Validation of serum miRNA expressions using AD postmortem brains. Total RNA was isolated from postmortem brains (frontal cortex) of AD patients at Braak stages IV (n=4), V (n=6), and VI (n=6), and in controls (n=5). Expression of selected 5-miRNA panel was quantified by qRT-PCR. The average fold change in each miRNA was higher in AD brains at Braak stages IV, V, and VI compared to the control brains. Expression of miR-455-3p was increased in the AD brains at all Braak stages compared to controls. However, a significant upregulation was observed in brains from AD patients at the Braak stage V (26.59-fold, P=0.016) (FIG. 3A). Similarly, miR-3613-3p expression was also higher in the brain tissues at Braak stage V (P=0.003) compared to controls (FIG. 3B).

MiR-4674 expression was also higher in the postmortem brains from AD patients at the Braak stages IV and V, but it was significantly higher at stage VI (P=0.003) (FIG. 3C). MiR-4668-5p expression was also up-regulated in AD brains at Braak stages IV, V, and VI, but not the significant level (FIG. 3D). Mir-6722 expression was down-regulated in AD serum samples. However, the expression of mir-6722 was surprisingly increased in the AD patients at Braak stage VI (P=0.018) (FIG. 3E). The opposite facts were observed in the analysis of mir-6722 expression.

The upregulation of miR-455-3p was significant in postmortem brains from AD patients at Braak stage V. Interestingly, those individuals were all having the ApoE (3/4) genotype. The expression of miR-455-3p was the most significantly higher in both the AD serum and AD postmortem brains suggesting that it might be implicated in AD detection and pathogenesis.

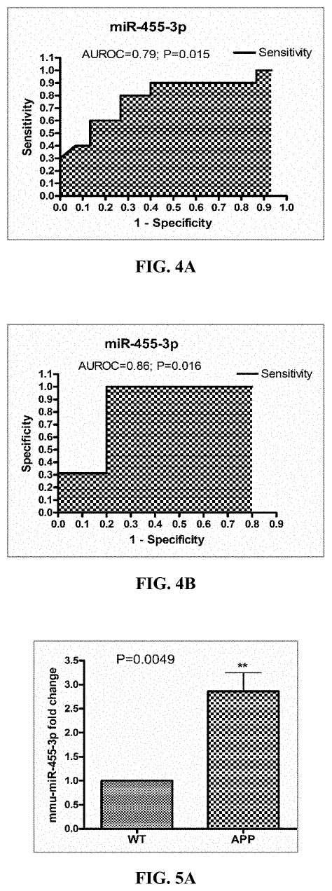

Receiver operating characteristics (ROC) curve analysis of miR-455-3p. To determine the diagnostic accuracy using miRNAs in AD patients, ROC curves analysis was studied for miR-455-3p expressions in serum and AD brain samples. The curves were plotted, based on the ΔCt value of miR-455-3p expression in serum samples from the AD patients (n=11) and controls (n=18). Upon analysis, miR-455-3p showed significant area under curve (AUC). The AUROC=0.79 with a 95% confidence interval was 0.59 to 0.98 (P=0.015) in AD serum samples compared to the healthy controls (FIG. 4A). Further, ROC analysis of miR-455-3p expression in postmortem brains from 16 AD patients and 5 healthy controls indicated the significant AUROC value of 0.86 (95% confidence interval was 0.61 to 1.11, P=0.016) (FIG. 4B). Thus, ROC analysis confirmed miR-455-3p as a valuable molecule capable of discriminating persons with and without AD.

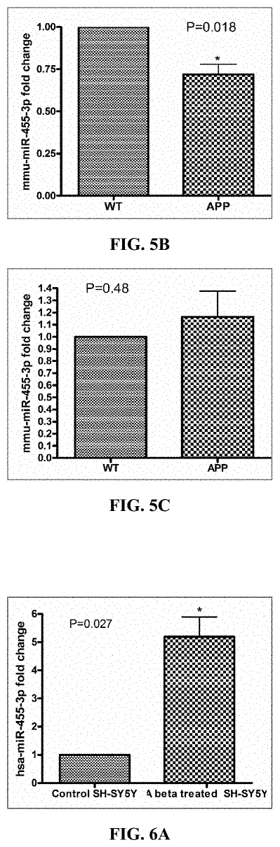

Expression of miR-455-3p in APP transgenic mice. Since miR-455-3p showed promising results in terms of its expression in AD serum samples and AD postmortem brains, miR-455-3p expression was also studied in the cortical tissues from an APP transgenic mouse model of AD (Tg2576 line). This study investigated the mmu-miR-455-3p expression in brain tissues from 6-month-old APP mice (n=6) and C57BL/6 wild-type mice (n=6). Total RNA was extracted from disease-affected tissue from the cerebral cortex and non-affected-cerebellum, and mmu-miR-455-3p expression was measured by qRT-PCR. Results showed a 1.8-fold (P=0.004) upregulation of mmu-miR-455-3p in the cerebral cortex tissues of the APP mice, compared to the wild type mice (FIG. 5A). Interestingly, in cerebellum, mmu-miR-455-3p expression was significantly (P=0.018) reduced in the APP mice (FIG. 5B). Expression of mmu-miR-455-3p was also examined in the serum samples from APP mice. Mmu-miR-455-3p expression was higher in the APP mice serum compared to the wild-type mice, although it was not statistically significant (FIG. 5C). A high level of mmu-miR-455-3p expression in the APP mice confirmed a possible role in Aβ-mediated AD pathogenesis.

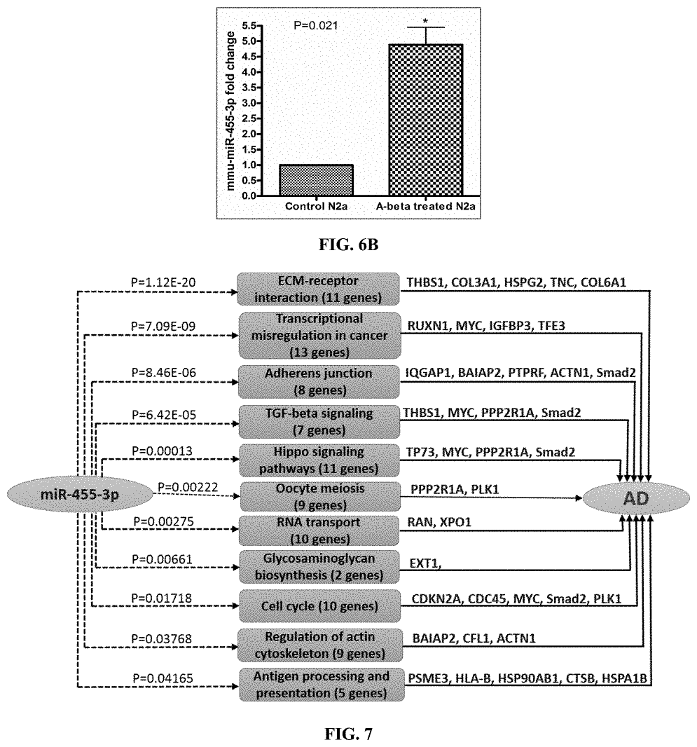

MiR-455-3p expression in Aβ(1-42) treated cells. To determine the effects of the Aβ on the expression of miR-455-3p, the SH-SY5Y (human neuroblastoma) and N2a (mouse neuroblastoma) cells were treated with the Aβ(1-42) peptide (20 μM) for 6 hours. Total intracellular RNA was extracted and expression of human and mouse miR-455-3p were measured by qRT-PCR. Results showed a 4.1-fold (P=0.027) increase in hsa-miR-455-3p expression in the Aβ-treated SH-SY5Y cells compared to control (untreated) cells (FIG. 6A). Similarly, in N2a cells, mmu-miR-455-3p expression was also upregulated by 3.8-fold (P=0.021) in Aβ-treated cells compared to control cells (FIG. 6B). These results further confirmed the significant response of miR-455-3p in Aβ pathologies.

MiRNA-associated signaling pathways. MiRNA-associated signaling pathways were analyzed using DIANA TOOLS-miRPath algorithm to identify the biological function of these miRNAs and their role in AD pathogenesis. MicroT-CDS files for all five miRNAs were run with a p value<0.05. KEGG pathway analysis unveiled more than 54 biological pathways associated with these miRNAs. This analysis focused on the miR-455-3p and identified possible molecular targets involved in AD pathogenesis. The miRNA analysis identified the relationship of miR-455-3p with 11 biological pathways and associated genes (FIG. 7). The most important signaling pathways were: the ECM-receptor interaction, the adherens junction, the TGF-beta signaling pathway, the hippo signaling pathway, and the regulation of the actin cytoskeleton. These signaling pathways and some of their genes (THBS1, COL3A1, HSPG2, COL6A1, RUXN1, MYC, Smad2, PLK1, and TNC) were directly associated with AD pathogenesis. The upregulation of miR-455-3p in AD development might be associated with the modulation of the above-mentioned genes. Thus, analysis of the signaling pathways revealed a possible molecular mechanism for how miR-455-3p is involved in AD pathogenesis.

Despite the enormous research efforts that have gone into developing ways to diagnose AD at the earliest stages possible, little progress has been made. Perhaps this is at least in partly due to initial research that focused on personal characteristics of persons who developed AD, such as their life style, body mass index, status of AD-related alleles, their genotypic and phenotypic variations, and environmental factors (11,23,24). This research recently broadened the list of potential biomarkers for AD to include blood-based miRNAs, but until the last 5-8 years, little research was actually conducted to narrow the list of miRNAs that might serve as biomarkers for AD, since more than one hundred miRNAs were found to be deregulated in AD patients (6,11,14-21).

The present study narrows the field of miRNAs that might serve as peripheral biomarkers for AD. Using Affymetrix microarray analysis, the inventors identified about 6631 types of small RNAs in the serum of patients with AD and with MCI. Of these, only 2578 were mature, and 2025 were stem-loop precursors of human miRNAs. These numbers of mature miRNAs were almost same as the miRNAs entry on miRbase release 21 (2588 mature) for Homo sapiens (www.mirbase.org/cgi-bin/browse.pl). From these results, the inventors came to know that most of genomic miRNAs were present and/or secreted in peripheral circulation. Based on a recent literature of circulatory miRNAs (11), the inventors had expected that disease-specific miRNAs or miRNAs associated with a particular pathological state, such as AD, were differently expressed and released in peripheral circulation (11). When the inventors compared the 3 different study groups in this study (serum from AD patients, MCI subjects and healthy controls), it was found that miRNA expressions of a wide number of miRNAs change, depending on the serum-donor's stage of disease progression. The highest variation of miRNA expressions was found in the patients' serum who were at the initial stage of disease progression, when the diagnosis of these patients went from control to MCI. Hence, disease-specific early physiological changes are crucial for the miRNAs deregulation in cells. Sequencing analysis of serum exosomes unveiled differential expression of 17 miRNAs in the serum from 3 subject groups (17). However, due to low number of MCI subjects (n=11), none of the exosomal miRNA was verified as biomarker for disease progression (17). In present study, five miRNAs (miR-455-3p, miR-4668-5p, miR-3613-3p, miR-4674, and mir-6722) were selected for validation in order to determine potential biomarker. Results showed a remarkable variation of miR-455-3p in AD serum samples, AD postmortem brains and AD mice, suggesting that it is potential biomarker for AD. These five miRNAs are known to have specific regulating roles in different diseases. MiR-455-3p has a role in colon cancer (25) and also participates in chondrogenic differentiation (26) and cartilage development and degeneration (27). In patients with preeclampsia, miR-455-3p was also found to be linked with hypoxia signaling and the regulation of brown adipogenesis via the HIF1an-AMPK-PGC1a signaling network (28,29). In familial amyotrophic lateral sclerosis, downregulation of miR-455-3p expression has been reported in the sera of these patients (30). The roles of miR-3613-3p and miR-4668-5p have been studied in the pathogenesis and progression of IgA nephropathy (31) and in mesial temporal lobe epilepsy (32). In the plasma of AD patients, low levels of miR-3613-3p were detected using RNA sequencing (33). However, current study showed increased levels of miR-3613-3p in the serum samples of AD patients. In hemolysis-free blood plasma from prostatic cancer patients, an increased level of miR-4674 was reported (34). However, role of these miRNAs is not widely reported in AD and other neurodegenerative diseases.

A recent analysis of biofluids (serum, plasma, CSF) from AD patients revealed many miRNA potential biomarkers for AD, such as miR-9, miR-125b, miR-146a, miR-181c, let-7g-5p, and miR-191-5p (11). However, their expression levels and molecular characterizations were not investigated using postmortem brains from AD patients and AD cell and mouse models. Consequently, no miRNA has been identified as the most likely biomarker for AD. In this current study, the inventors analyzed sera and cortices from AD patients found a significant upregulation of miR-455-3p. The inventors attempted to replicate these observations in AD postmortem brains, APP transgenic mice, and AD cell lines. Interestingly, miR-455-3p expression was more significantly up-regulated in the brains and sera from AD patients at Braak stage who had the ApoE (3/4) genotype. These observations unveiled a possible molecular interaction between miR-455-3p and the ApoE4 genotype.

Another significant finding was that APP transgenic mice exhibited Aβ pathologies (35,36) that corresponded to their high expression of miR-455-3p in the disease-affected brain cortex, but not in other brain areas known not to be affected by disease, such as the cerebellum. These findings indicate a possible molecular link between APP processing and miR-455-3p. This hypothesis was further tested on human and mouse neuroblastoma cells treated with toxic Aβ(1-42) peptides which mimics the AD type pathophysiology. Higher expression levels of miR-455-3p in the Aβ(1-42)-treated cells further support miR-455-3p as a potential biomarker for AD. This study is the first to identify miR-455-3p as a key molecule expressing biomarker properties for AD.

MiR-455-3p expression is regulated by the transforming growth factor beta (TGF-β) (37), and its level of expression has been found to be induced by TGF-β1, TGF-β3, and activin A in human SW-1353 chondrosarcoma cells and murine C3H10T1/2 cells (28). The TGF-β signaling pathway reported to play a critical role in Aβ processing in patients with AD since reduced TGF-signaling has been found to be increased in Aβ deposits in patients with AD (38,39).

Through pathway analysis, the KEGG pathway was found to regulate the TGF-β signaling pathway and eleven associated genes by miR-455-3p (FIG. 7). As a consequence, miR-455-3p is interconnected with TGF-β signaling and Aβ synthesis, and hence, may play a crucial role in AD pathogenesis. MiR-455-3p might also have a major regulatory role in other cellular pathways through modulation of their genes in AD pathogenesis (FIG. 7). It is possible that miR-455-3p may be involved in AD progression through altered expressions of HSPG2, THBS1, COL3A1, COL6A1, TNC, MYC, Smad2, RAN, PLK1, TP73, ACTN1, and IQGAP1 genes (39-47).

FIG. 8 shows MicroRNA 455-3p as a potential peripheral biomarker for Alzheimer's disease. To identify the early peripheral microRNA (miRNA) biomarkers for Alzheimer's disease (AD), the present inventors used different sources of materials in this study. Serum samples and postmortem brains from AD patients, brain tissues from AD transgenic mice and AD cell lines were used. Primary screening of serum samples from AD patients, MCI individuals and healthy subjects showed deregulated expression of five miRNAs by global Affymetrix based microarray analysis. MiRNAs were further validated in serum samples form AD patients, MCI individuals and healthy controls. Out of five, only miR-455-3p showed the most significant upregulation in AD and MCI cases compared to controls. Postmortem brains form AD patients also showed the significant upregulation of miR-455-3p in AD cases compared to controls. miR-455-3p expression also upregulated in the frontal cortices of APP transgenic mice compared to wild type mice. Finally, the inventors tested the miR-455-3p expression in human and mouse neuroblastoma cell lines treated with Amyloid-beta 42 peptide. In Amyloid-beta 42 treated cells, miR-455-3p expression was also elevated compared to control cells.

FIG. 9 shows the details of a microRNA 455-3p study. Lymphoblasts and fibroblasts from AD patients and healthy controls (obtained from NIA cell line repositories) can be used in order to establish miR-455-3p as peripheral biomarker for Alzheimer's disease. Expression of miR-455-3p can be correlated with the levels of AD proteins. The molecular properties of miR-455-3p can also be used to mimic (overexpression) and (inhibitor) knockout approaches in human and mouse neuronal cells. These experiments can be used to determine the deleterious and protective effects of miR-455-3p in AD neurons. Using overexpression and/or knockouts of miR455-3p in APP transgenic and knockout mice it is possible to study the molecular features of miR455-3p during disease progression of AD. Finally, miR-455-3p transgenic and miR-455-3p knockout mice lines can be generated and used to explore the role of miR-455-3p in maintaining synaptic, mitochondrial and inflammatory functions and cognitive behavior.

In summary, the present inventors have identified multiple differentially expressed circulatory miRNAs in AD patients and subjects with MCI relative to healthy controls. A careful validation of differentially expressed miRNAs using AD postmortem brains, APP transgenic mice and AD cell lines revealed that miR-455-3p could be a potential diagnostic biomarker for AD. Further research is still needed to better understand the role of miR-455-3p in AD progression and pathogenesis.

Study subjects. Sera and DNA samples were collected from patients under the FRONTIERS project based at Garrison Institute on Aging (GIA), Texas Tech University Health Sciences Center (TTUHSC). These samples were obtained from 11 patients diagnosed with AD, 20 patients with MCI, and 18 healthy controls. The study protocol was approved by the Institutional Review Board for FRONTIERS, and all subjects provided informed written consent. All the bio-specimens were stored at GIA Bio-Bank. Patient information on demographic characteristics, medical history, biochemical profiles, and their risk factors for AD was gathered with a standardized questionnaire. Demographic and clinical characteristics of subjects are listed in the Table 3. After completing the questionnaire, all subjects underwent a detailed clinical examination to evaluate them for inclusion and exclusion criteria established by NINCDS-ADRDA. The inclusion criteria were: (1) 45 years and above age, (2) rural community based West Texas individuals, and (3) all study participants have assessed for cognitive functions. The exclusion criteria were: (1) individuals with strong medication and (2) too many health complications.

MiRNAs extraction. MiRNAs, including other small RNAs, were extracted from the serum samples with the miReasy serum/plasma kit (Qiagen, Germany) as per manufacturer's instructions (16). Briefly, 200 μl of serum samples were mixed with 5 volumes of a Qiazol lysis reagent and an equal volume of chloroform, and centrifuged to separate the aqueous phase. MiRNAs accumulated in the aqueous phase were precipitated with 100% ethanol. MiRNAs were washed with a buffer and 80% ethanol, and purified miRNAs were eluted in 15 μl of RNase-free water. RNA quality and quantity were measured by NanoDrop2000c (Thermo Scientific, USA).

Primary miRNAs screening by Affymetrix microarray. Detailed miRNAs screening of the serum samples were conducted in the University of Texas Southwestern Medical Center, Genomics and Microarray Core Facility, Dallas. The miRNA expression profiles were generated with Affymetrix GeneChip miRNA array v. 4.0 (Affymetrix). The GeneChip miRNA 2.0 arrays contain a 100% miRBase version 20 coverage: 30,424 mature miRNAs were from all organism; 5,214 from human, rat, and mouse miRNAs; and 1,996 from human snoRNA and scaRNA. It also provided 3,770 probe sets that are unique to human, mouse, and rat pre-miRNA hairpin sequences. The GeneChip miRNA 4.0 array demonstrated superior performance with 0.95 reproducibility (inter- and intra-lot) and >80% of transcripts were detected at 1.3 amol from 130 ng of total RNA. Data were represented by the GeneChip miRNA 4.0 array in 4 logs that correlated with a dynamic range of >0.97 signal and >0.94 fold-change.

Briefly, 8 μl of total RNA was treated for poly (A) tailing reaction at 37° C. for 15 min as per the protocol. 4 μl of 5× Flash Tag Biotin HSR ligation mix was added to poly (A) tailed RNA, and the mixture was incubated at 25° C. for 30 min, using the Flash Tag Biotin HSR Labeling kit following manufacturer's instructions (cat. no. HSR30FTA; Genisphere, LLC, Hatfield, Pa., USA). Biotin HSR that labeled with RNA was mixed with an array hybridization cocktail according to the GeneChip Eukaryotic Hybridization control kit manual and was processed using the Affymetrix GeneChip miRNA array. Samples were incubated on the hybridization array chip at 48° C. and 60 rpm for 16 to 18 hours. After hybridization, the chips were washed and stained by GeneChip hybridization, washed again, and then stained with an Affymetrix kit according to the manufacturer's protocols. The hybridized chips were scanned with an Affymetrix GCS 3000 7G Scanner (48).

Microarray data analysis. Raw data were obtained, using the Affymetrix GeneChip array in the form of an individual CHP file. Each sample was then analyzed, using Transcriptome Analysis Console software v. 3. Tukey's bi-weight average (log 2) intensity was analyzed with an ANOVA p-value (<0.05) and FDR p-value (<0.05) for all conditions, for all genes in the samples from AD, MCI and control group. SAM (significance analysis of microarray) with the R package was used to identify differentially expressed miRNA and gene probe sets in samples from the AD patients and the controls. Probe sets were considered biologically significant if the fold changes were 2 (49).

Validation of serum miRNAs expression using qRT-PCR. (i) Polyadenylation—One μg of total RNA was polyadenylated with an miRNA First-Strand cDNA synthesis kit (Agilent Technologies Inc., CA, USA), following manufacturer's instructions. Briefly, a polyA reaction was prepared by mixing RNA with 4.0 μl of 5× poly A polymerase buffer, 1.0 μl of rATP (10 mM), 1 μl of E. coli poly A polymerase, producing a final volume of 20 μl with RNase-free water. The tube with these components was incubated at 37° C. for 30 min, followed another incubation at 95° C. for 5 min to terminate the adenylation reaction (50). (ii) cDNA synthesis—Ten μl of polyadenylated miRNAs were processed for cDNA synthesis with the miRNA First-Strand cDNA synthesis kit (Agilent Technologies Inc.). The following reaction components were combined in a tube: 2 μl of 10× AffinityScript RT buffer, 0.8 μl of dNTP mix (100 mM), 1 μl of RT adaptor primer (10 μM), 1.0 μl of AffinityScript RT/RNase Block enzyme, and polyadenylated RNA. The combination resulted in a reaction volume of 20 μl RNase-free water. This reaction mixture was incubated at 55° C. for 5 min, then at 25° C. for 15 min, followed by an incubation at 42° C. for 30 min, and a final incubation at 95° C. for 5 min in a Veriti 96 well thermal cycler (Applied Biosystems, USA). Resulting cDNAs were diluted with 20 μl of RNase-free water and stored at 80° C. for further analysis. (iii) qRT-PCR for miRNAs—qRT-PCR reaction was performed by preparing a reaction mixture containing 1 μl of miRNA-specific forward primer (10 μm), 1 μl of a universal reverse primer (3.125 μm) (Agilent Technologies Inc., CA, USA), 10 μl of 2×SYBR® Green PCR master mix (Applied Biosystems, NY, USA), and 1 μl of cDNA. To this mixture RNase-free water was added up to a 20 μl final volume. Primers for hsa-miR-455-3p, miR-4674, miR-3613-3p, miR-4668-5p, and mir-6722 were synthesized commercially (Integrated DNA Technologies, Inc. Iowa USA) (Table 3).

TABLE 3
miRNAs primers sequences for qRT-PCR

| SEQ | | | |
|---|---|---|---|
| ID | | | Base |
| NO: | miRNA | Sequence (5′ to 3′) | pairs |
| 1 | Hsa-miR-455-3p | F-GCAGTCCATGGGCATATACAC | 21 |
| 2 | Mmu-miR-455-3p | F-GCAGTCCACGGGCATATACAC | 21 |
| 3 | Hsa-miR-4674-3p | F-CTGGGCTCGGGACGCGCGGCT | 21 |
| 4 | Hsa-miR-4668-5p | F-AGGGAAAAAAAAAAGGATTTGTC | 23 |
| 5 | Hsa-miR-3613-3p | F-ACAAAAAAAAAAGCCCAACCCTTC | 24 |
| 6 | Hsa-mir-6722 | F- GGCCTCAGGCAGGCGCACCCGA | 22 |
| 7 | | R- GGGTGGGCCAGGCTGTGGGGCG | 22 |
| 8 | U6 snRNA | F- CGCTTCGGCAGCACATATACTAA | 23 |
| 9 | | R- TATGGAACGCTTCACGAATTTGC | 23 |
| 10 | snoRNA-202 | F-AGTACTTTTGAACCCTTTTCCA | 22 |

To normalize the miRNA expression, U6 snRNA (small nuclear RNA) expression was also quantified in the serum samples, which was used as an internal control. The reaction mixture of each sample was prepared in triplicates. The reaction was set in the 7900HT Fast Real Time PCR System (Applied Biosystems, USA) using following reaction conditions: initial denaturation at 95° C. for 5 min, denaturation at 95° C. for 10 sec, annealing at 60° C. for 15 sec, and extension at 72° C. for 25 sec. The relative levels of miRNAs in the AD patients versus the controls and versus the MCI subjects were determined in terms of their fold change, using the formula (2^{-ΔΔCt}), where ΔCt was calculated by subtracting Ct of U6snRNA from the Ct of particular miRNAs, and ΔΔCt value was obtained by subtracting ΔCt of particular miRNAs in the controls from the ΔCt of miRNAs in the AD and MCI. qRT-PCR was performed in triplicate, and the data were expressed as the mean±SD (50,51).

Postmortem brains from AD patients. Postmortem brain tissues were obtained from the GIA Brain Bank. The frontal cortices of the postmortem brains were dissected from the AD patients (n=16) and controls (n=5). Demographic details of study participants were given in Table 4. The study protocol was approved by the Institute Ethical Committee at TTUHSC, and brain tissue was obtained after written informed consent from the deceased's relatives.

TABLE 4
Characteristics of postmortem brain tissues from controls and AD patients

| Cases | Gender | Age (yrs) | Braak Stage | PMI (hrs) | ApoE |
|---|---|---|---|---|---|
| HC1 | M | 71 | — | 9 | ¾ |
| HC2 | M | 68 | — | 6.5 | ¾ |
| HC3 | F | 72 | — | 11.5 | 3/3 |
| HC4 | F | 71 | — | 7.5 | 2/2 |
| HC5 | M | 82 | — | 6.25 | 3/3 |
| AD1 | M | 82 | IV | — | ¾ |
| AD2 | M | 62 | IV | 5 | 3/3 |
| AD3 | M | 78 | IV | 7.5 | ¾ |
| AD4 | M | 91 | IV | 8 | 3/3 |
| AD5 | F | 77 | V | 4 | ¾ |
| AD6 | F | 86 | V | 3.5 | ¾ |
| AD7 | F | 86 | V | 5 | ¾ |
| AD8 | F | 75 | V | 4 | ¾ |
| AD9 | F | 80 | V | 5 | ¾ |
| AD10 | M | 78 | V | 7 | ¾ |
| AD11 | M | 74 | VI | 7 | ¾ |
| AD12 | F | 81 | VI | 6.25 | 3/3 |
| AD13 | F | 83 | VI | 9.25 | ¾ |
| AD14 | F | 86 | VI | 6 | ¾ |
| AD15 | M | 84 | VI | 8 | 3/3 |
| AD16 | M | 82 | VI | 5.25 | ¾ |

MiRNAs extraction and qRT-PCR. MiRNAs extraction and cDNA synthesis were followed as described above, while total RNA was isolated from the 80 mg of frontal cortices using the TriZol RT reagent (Ambion, USA) as per manufacturer instructions. Briefly, tissue samples were homogenized in 1 ml of TriZol reagent with Bio-Gen PRO200 Homogenizer (PRO Scientific Inc., CT, USA) in a 2-ml RNase-free tube. Chloroform (0.2 ml) was added to the tissue homogenate, vigorously shaken for 15 seconds, and stored for 5 min at room temperature. The mixture was then centrifuged at 12,000 g for 15 min at 4° C. The supernatant was transferred to a new tube and precipitated with 0.5 ml of isopropanol for 15 min at room temperature. Samples were centrifuged at 12,000 g for 10 min at 4° C. The resulting RNA pellet was washed with 1 ml of 75% ethanol and centrifuged at 7,500 g for 5 min at 4° C. The RNA pellet was dried and dissolved in 20 μl of DEPC-treated water. The quality and quantity of the RNA were analyzed by NanoDrop analysis. The value of absorbance of each brain RNA sample (A₂₆₀/A₂₈₀) was 1.8 to 2.0. cDNA was synthesized from 1 μg of RNA using miRNA First-Strand cDNA synthesis kit (Agilent Technologies Inc.). qRT-PCR were analyzed for miR-455-3p, miR-4674, miR-3613-3p, miR-4668-5p, and mir-6722 as described previously.

Animal models. Amyloid-β transgenic (APP) mice were generated with the mutant human APP gene 695-amino-acid isoform and a double mutation (Lys670Asn and Met671Leu) (35). The APP mouse model exhibits age-dependent Aβ plaques as well as a distribution of Aβ plaques in the cerebral cortex and the hippocampus, but not in the striatum, the deep gray nuclei, and the brain stem. Disease in this mouse model parallels AD in that elevated amounts of soluble Aβ correlate with increased free-radical production, and the Aβ plaques evoke a microglial reaction in their immediate vicinity. Cerebral cortex tissues were collected from 6-month-old APP transgenic mice (n=6) and age-matched, non-transgenic wild-type mice (n=6). To determine transgene-positive mice to model the human APP, genotyping was performed in accordance with the TTUHSC Policy on Genotype Tissue Collection, using the DNA prepared from tail biopsy and PCR amplification (35). All mice were observed daily by a veterinary caretaker and also examined twice weekly by laboratory staff. If any mice showed premature signs of neurological deterioration, they were euthanized before experimentation according to euthanasia procedure approved by the TTUHSC-IACUC and were not used in the study.

Amyloid-β(1-42) treatment to cell lines. Human neuroblastoma (SH-SY5Y) and mouse neuroblastoma (N2a) cell lines were purchased from American Tissue Type Collection (ATCC) (Virginia, USA). Cells were grown in a medium (1:1) Dulbecco's modified eagle's medium and minimum essential medium, 5% fetal bovine serum, 1× penicillin and streptomycin) at 37° C. in a humidified incubator with a 5% CO₂ environment. After the cells were seeded, they were allowed to grow for 24-48 hours or until 80% confluence in 6-well plates. They were then used for experimentation. Two different groups of cells were used: (1) untreated SH-SY5Y/N2a cells and (2) Aβ(1-42) peptide treated SH-SY5Y/N2a cells. They were incubated with the Aβ(1-42) peptide (20 mM final concentration) in triplicate for 6 hours. Both groups of cells were harvested after treatment and processed for total RNA extraction and miR-455-3p quantification.

MiRNAs pathway analysis. MiRNAs that were associated with signaling pathways were analyzed with the miRPath v3.0 web server algorithm (52). Briefly, species was defined as ‘human, mouse’ and miR-455-3p, miR-4674, miR-3613-3p, miR-4668-5p, and mir-6722 were entered. MiRNAs that target genes and biological pathways were analyzed, using microT-CDS and TarBase to classify the GO category, the P<0.05 of the KEGG pathway enrichment, and the microT-CDS threshold (0.8). The miRNA-targeted genes in different KEGG molecular pathways were ranked according to their P-value. The false discovery rate (FDR) P<0.05 was considered statistically significant.

Statistical analysis. The qRT-PCR validation analysis was based on the 2\hat{ }−ΔΔCT value of genes in each sample from AD, MCI subjects and controls. Statistical analysis was performed with Prism software, v, 6 (La Zolla, Calif.). P-value was calculated, based on the paired and unpaired t-tests for analyzing 2 groups and using one-way comparative analysis of variance (ANOVA) when comparing between more than 2 groups. P<0.05 was considered statistically significant.

### Example 2

The goal of present study was to identify a suitable, non-invasive, blood-based early biomarker for AD detection. To achieve this goal, the inventors focused on circulatory microRNAs (cmiRNAs), which are quite stable in peripheral circulation and levels of particular miRNA seems to be changing with disease severity. The previous research findings on human serum samples from AD patients, MCI individual and healthy subjects identified significant number of deregulated miRNAs in patients compared to controls (Kumar et al., 2017). A few of them were significantly upregulated and some were down regulated in AD and MCI individual compared to healthy controls. One of the most suitable identified candidate in the study was microRNA-455-3p. Expression of miR-455-3p was found to be significantly upregulated in AD serum samples, AD postmortem brains, AD mouse model, and AD cell lines (Kumar et al., 2017). Upregulation of miR-455-3p in different cell and mouse models of AD proven its biomarker potential for AD. To further strengthen the findings, the present study is focused on the AD postmortem brains obtained from NIH NeuroBioBanks, human fibroblast, and B-lymphocytes cell lines derived from familial AD and sporadic AD patients. Expression of miR-455-3p was quantified and its diagnostic potential was examined in different sources. Further, in-silico analysis was performed to understand the roles and downstream application of miR-455-3p in AD. Findings from this study, will provide the valuable information about miR-455-3p role in AD and in search of pre-clinical biomarker for early AD detection.

Study Subjects. (a) AD postmortem brains—Postmortem brains from AD patients and healthy controls were obtained from three NIH NeuroBioBanks—(1) Human Brain and Spinal Fluid Resource Center, 11301 Wilshire Blvd (127A), Los Angeles, Calif. (2) Brain Endowment Bank, University of Miami, Millar School of Medicine, 1951, NW 7th Avenue Suite 240, Miami, Fla. (3) Mount Sinai NIH Brain and Tissue Repository, 130 West Kingsbridge Road Bronx, N.Y. Brain tissues were dissected from the Brodmann's Area 10 of the frontal cortices from AD patients (n=27) and age and sex matched healthy controls (n=15). Demographic and clinical details of study specimens were given in Table 5.

TABLE 5
Demographic and clinical details of the brain samples

| | | | | Neuro | | Autolysis |
|---|---|---|---|---|---|---|
| S. No | Sample ID | Age | Sex | pathology | Structure | time |
| 1 | 4130 | 67 | F | Control | Broadmann's Area 10 | 11.8 |
| 2 | 4431 | 68 | F | Control | Broadmann's Area 10 | 23.7 |
| 3 | 4660 | 73 | F | Control | Broadmann's Area 10 | 18.5 |
| 4 | 5072 | 83 | M | Control | Broadmann's Area 10 | 19.5 |
| 5 | 5190 | 68 | M | Control | Broadmann's Area 10 | 20.3 |
| 6 | HCT15HAO1713 | 70 | M | Control | Broadmann's Area 10 | 12.7 |
| 7 | HCTZZC1711 | 82 | F | Control | Broadmann's Area 10 | 14.2 |
| 8 | HCT15HBC1709 | 83 | M | Control | Broadmann's Area 10 | 25 |
| 9 | HCTZZT1702 | 84 | M | Control | Broadmann's Area 10 | 15.5 |
| 10 | HCT15HBU1704 | 91 | F | Control | Broadmann's Area 10 | 18.7 |
| 11 | 77428 | 65 | M | Control | Broadmann's Area 10 | 3.8 |
| 12 | 77431 | 103 | F | Control | Broadmann's Area 10 | 3.8 |
| 13 | 77433 | 75 | M | Control | Broadmann's Area 10 | 5 |
| 14 | 77436 | 93 | M | Control | Broadmann's Area 10 | 4.1 |
| 15 | 77437 | 84 | F | Control | Broadmann's Area 10 | 5.4 |
| 16 | 4513 | 74 | M | AD | Broadmann's Area 10 | 15.6 |
| 17 | 4498 | 76 | M | AD | Broadmann's Area 10 | 12.9 |
| 18 | 4204 | 68 | M | AD | Broadmann's Area 10 | 11.9 |
| 19 | 4203 | 72 | F | AD | Broadmann's Area 10 | 20.3 |
| 20 | 4454 | 82 | F | AD | Broadmann's Area 10 | 9 |
| 21 | 4043 | 80 | F | AD | Broadmann's Area 10 | 13 |
| 22 | 4382 | 74 | F | AD | Broadmann's Area 10 | 16.2 |
| 23 | 4617 | 73 | F | AD | Broadmann's Area 10 | 18.9 |
| 24 | 4718 | 93 | F | AD | Broadmann's Area 10 | 8.2 |
| 25 | 4608 | 80 | M | AD | Broadmann's Area 10 | 3.1 |
| 26 | 4752 | 89 | M | AD | Broadmann's Area 10 | 9 |
| 27 | 4788 | 65 | M | AD | Broadmann's Area 10 | 7.8 |
| 28 | HBFR1703 | 69 | F | AD | Broadmann's Area 10 | 22 |
| 29 | HBFQ1711 | 77 | M | AD | Broadmann's Area 10 | 18 |
| 30 | HBJG1710 | 79 | M | AD | Broadmann's Area 10 | 23.8 |
| 31 | HBDA1704 | 80 | M | AD | Broadmann's Area 10 | 22.1 |
| 32 | HCTYN1713 | 80 | F | AD | Broadmann's Area 10 | 6.5 |
| 33 | HBDI1710 | 85 | F | AD | Broadmann's Area 10 | 8 |
| 34 | HBEM1701 | 86 | M | AD | Broadmann's Area 10 | 15.5 |
| 35 | HBIP1701 | 90 | F | AD | Broadmann's Area 10 | 22.1 |
| 36 | HBCG1703 | 90 | F | AD | Broadmann's Area 10 | 8.5 |
| 37 | HCTZX1702 | 95 | M | AD | Broadmann's Area 10 | 19.8 |
| 38 | 77423 | 79 | F | AD | Broadmann's Area 10 | 6.5 |
| 39 | 77424 | 69 | M | AD | Broadmann's Area 10 | 5.4 |
| 40 | 77425 | 75 | M | AD | Broadmann's Area 10 | 8 |
| 41 | 77426 | 94 | F | AD | Broadmann's Area 10 | 4.3 |
| 42 | 77427 | 82 | M | AD | Broadmann's Area 10 | 20.6 |

(b) AD patients cell lines—Human skin fibroblast and Lymphoblast cell culture systems were used for these studies. Banked skin fibroblasts and lymphoblast cells with the diagnoses AD, non-AD dementia (e.g., Huntington's disease and Parkinson's disease, and schizophrenia), and age-matched control were obtained from the Coriell Institute of Medical Research, Camden, N.J., USA. The demographic details of cell lines along with their passage numbers, biopsy sources and tissue types were provided in Table 6. Cells were cultured and maintained in RPMI1640 for B-lymphocytes and MEM media for Fibroblasts (Life Technologies Corporation, NY, USA; supplemented with 10% Fetal Bovine Serum and 1× penicillin/streptomycin) at 37° C. with 5% CO₂ to the 90-100% confluence stage in 25 and 75 cm² cell culture flasks.

TABLE 6
Details of human Fibroblasts and B-lymphocytes

| | | | | | | | | |
|---|---|---|---|---|---|---|---|---|
| | | | | Age | Biopsy | Tissue | | Disease |
| S. No. | Catalog no | Passage no | Sex | (Years) | sources | type | Race | status |

(A) Fibroblasts

| | | | | | | | | |
|---|---|---|---|---|---|---|---|---|
| 1 | AG02261 | 11 | M | 61 | Abdomen | Skin | Caucasian | Healthy |
| | | | | | | | | control |
| 2 | AG16104 | 6 | F | 55 | Arm | Skin | Black | Healthy |
| | | | | | | | | control |
| 3 | AG16086 | 6 | F | 67 | Arm | Skin | Other | Healthy |
| | | | | | | | | control |
| 4 | AG12207 | 13 | M | 68 | Arm | Skin | NA | Healthy |
| | | | | | | | | control |
| 5 | AG02258 | 6 | F | 46 | Lung | Lung | Caucasian | Healthy |
| | | | | | | | | control |
| 6 | AG02262 | 4 | M | 61 | Lung | Lung | Caucasian | Healthy |
| | | | | | | | | control |
| 7 | AG06561 | 5 | F | 16FW^{#} | Sacrum | Skin | Caucasian | Healthy |
| | | | | | | | | control |
| 8 | AG12211 | 11 | M | 54 | Lung | Lung | Caucasian | Healthy |
| | | | | | | | | control |
| 9 | AG05810 | 11 | F | 79 | Arm | Skin | Caucasian | Familial |
| | | | | | | | | AD |
| 10 | AG06844 | 12 | M | 59 | Arm | Skin | Caucasian | Familial |
| | | | | | | | | AD |
| 11 | AG07613 | 16 | M | 66 | Arm | Skin | Caucasian | Familial |
| | | | | | | | | AD |
| 12 | AG09908 | 14 | F | 81 | Arm | Skin | Caucasian | Familial |
| | | | | | | | | AD |
| 13 | AG04400 | 19 | F | 61 | Skin | Skin | Caucasian | Sporadic |
| | | | | | | | | AD |
| 14 | AG06263 | 11 | F | 67 | Arm | Skin | Caucasian | Sporadic |
| | | | | | | | | AD |
| 15 | AG06264 | 7 | F | 62 | Arm | Skin | NA | Sporadic |
| | | | | | | | | AD |
| 16 | AG07375 | 6 | M | 71 | Arm | Skin | Caucasian | Sporadic |
| | | | | | | | | AD |
| 17 | AG08243 | 7 | M | 72 | Arm | Skin | Caucasian | Sporadic |
| | | | | | | | | AD |
| 18 | AG11368 | 15 | M | 77 | Skin | Skin | Caucasian | Sporadic |
| | | | | | | | | AD |

(B) B-Lymphocytes

| | | | | | | | | |
|---|---|---|---|---|---|---|---|---|
| 1 | AG16639 | na | M | 77 | Peripheral | Blood | Caucasian | Healthy |
| | | | | | vein | | | control |
| 2 | AG11684 | na | M | 82 | Peripheral | Blood | Caucasian | Healthy |
| | | | | | vein | | | control |
| 3 | AG12034 | na | F | 80 | Peripheral | Blood | Caucasian | Healthy |
| | | | | | vein | | | control |
| 4 | AG11716 | na | M | 98 | Peripheral | Blood | Caucasian | Healthy |
| | | | | | vein | | | control |
| 5 | AG12032 | na | M | 84 | Peripheral | Blood | Caucasian | Healthy |
| | | | | | vein | | | control |
| 6 | AG16804 | na | F | 90 | Peripheral | Blood | Caucasian | Healthy |
| | | | | | vein | | | control |
| 7 | AG16927 | na | M | 85 | Peripheral | Blood | Caucasian | Healthy |
| | | | | | vein | | | control |
| 8 | AG16973 | na | F | 80 | Peripheral | Blood | Caucasian | Healthy |
| | | | | | vein | | | control |
| 9 | AG10673 | na | F | 85 | Peripheral | Blood | Black | Healthy |
| | | | | | vein | | | control |
| 10 | AG16907 | na | F | 88 | Peripheral | Blood | Caucasian | Healthy |
| | | | | | vein | | | control |
| 11 | AG08242 | na | M | 72 | Peripheral | Blood | Caucasian | Familial |
| | | | | | vein | | | AD |
| 12 | AG09905 | na | M | 72 | Peripheral | Blood | Caucasian | Familial |
| | | | | | vein | | | AD |
| 13 | AG09907 | na | F | 71 | Peripheral | Blood | Caucasian | Familial |
| | | | | | vein | | | AD |
| 14 | AG11755 | na | F | 85 | Peripheral | Blood | Caucasian | Familial |
| | | | | | vein | | | AD |
| 15 | AG11757 | na | F | 81 | Peripheral | Blood | Caucasian | Familial |
| | | | | | vein | | | AD |
| 16 | AG11758 | na | M | 83 | Peripheral | Blood | Caucasian | Familial |
| | | | | | vein | | | AD |
| 17 | AG06204 | na | M | 67 | Peripheral | Blood | Caucasian | Sporadic |
| | | | | | vein | | | AD |
| 18 | AG06868 | na | F | 60 | Peripheral | Blood | Caucasian | Sporadic |
| | | | | | vein | | | AD |
| 19 | AG11366 | na | M | 52 | Peripheral | Blood | Caucasian | Sporadic |
| | | | | | vein | | | AD |
| 20 | AG17512 | na | M | 70 | Peripheral | Blood | African | Sporadic |
| | | | | | vein | | American | AD |
| 21 | AG17529 | na | F | 86 | Peripheral | Blood | African | Sporadic |
| | | | | | vein | | American | AD |
| 22 | AG17574 | na | F | 83 | Peripheral | Blood | African | Sporadic |
| | | | | | vein | | American | AD |

na* not available

Ethical Approval and Consent. The study was conducted at the Garrison Institute on Aging (GIA), Texas Tech University Health Sciences Center (TTUHSC), and study protocol was approved by the Institutional Review Board of TTUHSC, Lubbock, Tex. for the use of biospecimens in Project FRONTIER (IRB #: L06-028). Regarding postmortem brains and cell lines used in the current study—each of the NIH NeuroBioBanks mentioned above operated under their institution's IRB approval. As determined by the FDA Research Involving Human Subjects Committee, current study did not reach the definition of “Human Subject Research” at 45 CFR 46.102(f) and thus, 45 CFR Part 46 does not apply (Ferguson et al., 2017). Further, according to Office for Human Research Protections Guidelines biospecimens obtained by the researchers from NIGMS Human Genetic Cell Repository are not considered to be human subjects because conducting research with the samples does not involve an intervention or interaction with the individual and the samples do not contain identifiable private information (www.coriell.org).

RNA Extraction—Total RNA was isolated from the 80 mg of frontal cortices using the TriZol RT reagent (Ambion, USA) as per manufacturer instructions. Briefly, tissue samples were homogenized in 1 ml of TriZol reagent with Bio-Gen PRO200 Homogenizer (PRO Scientific Inc., CT, USA) in a 2-ml RNase-free tube. Chloroform (0.2 ml) was added to the tissue homogenate, vigorously shaken for 15 s, and stored for 5 min at room temperature. The mixture was then centrifuged at 12,000 g for 15 min at 4° C. The supernatant was transferred to a new tube and precipitated with 0.5 ml of isopropanol for 15 min at room temperature. Samples were centrifuged at 12,000 g for 10 min at 4° C. The resulting RNA pellet was washed with 1 ml of 75% ethanol and centrifuged at 7,500 g for 5 min at 4° C. The RNA pellet was dried and dissolved in 50 μl of DEPC-treated water. The quality and quantity of the RNA were analyzed by NanoDrop analysis. The value of absorbance of each brain RNA sample (A260/A280) was 1.8-2.0.

Quantification of miRNAs Expression by Quantitative Real-Time PCR—Quantification Involved Three Steps:

Polyadenylation—One microgram of total RNA was polyadenylated with an miRNA First-Strand cDNA synthesis kit (Agilent Technologies Inc., CA, USA), following manufacturer's instructions. Briefly, a polyA reaction was prepared by mixing RNA with 4.0 μl of 5× poly A polymerase buffer, 1.0 μl of rATP (10 mM), 1 μl of E. coli poly A polymerase, producing a final volume of 20 μl with RNase free water. The tube with these components was incubated at 37° C. for 30 min, followed another incubation at 95° C. for 5 min to terminate the adenylation reaction (Kumar et al., 2014).

cDNA synthesis—Ten microliters of polyadenylated miRNAs were processed for cDNA synthesis with the miRNA First-Strand cDNA synthesis kit (Agilent Technologies Inc.). The following reaction components were combined in a tube: 2 μl of 10× AffinityScript RT buffer, 0.8 μl of dNTP mix (100 mM), 1 μl of RT adaptor primer (10 μM), 1.0 μl of AffinityScript RT/RNase Block enzyme, and polyadenylated RNA. The combination resulted in a reaction volume of 20 μl RNase-free water. This reaction mixture was incubated at 55° C. for 5 min, then at 25° C. for 15 min, followed by an incubation at 42° C. for 30 min, and a final incubation at 95° C. for 5 min in a Veriti 96 well thermal cycler (Applied Biosystems, USA). Resulting cDNAs were diluted with 20 μl of RNase-free water and stored at 80° C. for further analysis.

Real-time RT-PCR—Real-time RT-PCR reaction was performed by preparing a reaction mixture containing 1 μl of miRNA-specific forward primer (10 μm), 1 μl of a universal reverse primer (3.125 μm) (Agilent Technologies Inc., CA, USA), 10 μl of 2×SYBR Green PCR master mix (Applied Biosystems, NY, USA), and 1 μl of cDNA. To this mixture RNase-free water was added up to a 20 μl final volume. Primers for hsamiR-455-3p (Forward: 5′ GCAGTCCATGGGCATATACAC-3′ (SEQ ID NO: 1), and U6snRNA (P1: 5′-GCTTCGGCAGCACATATACTAA-3′ (SEQ ID NO: 8) and Reverse: 5′-TATGGAACGCTTCACGAATTTGC-3′ (SEQ ID NO: 9)) were synthesized commercially (Integrated DNA Technologies, Inc. Iowa USA). To normalize the miRNA expression, U6 snRNA (small nuclear RNA) expression was also quantified in the tissue and cells, which was used as an internal control. The reaction mixture of each sample was prepared in triplicates. The reaction was set in the 7900HT Fast Real Time PCR System (Applied Biosystems, USA) using following reaction conditions: initial denaturation at 95° C. for 5 min, denaturation at 95° C. for 10 s, annealing at 60° C. for 15 s, and extension at 72° C. for 25 s. The relative levels of miR-455-3p in the AD patients vs. the controls subjects were determined in terms of their fold change, using the formula (2−11Ct), where 1Ct was calculated by subtracting Ct of U6snRNA from the Ct of miR-455-3p. Real-time RT-PCR was performed in triplicate, and the data were expressed as the mean±SD (Kumar et al., 2014; Hamam et al., 2016).

In-Silico Analysis for miR-455-3p. MiR-455-3p target genes were analyzed using various on-line miRNA algorithms (diana-microt, microrna.org, mirdb, rna22-has, targetminer, and targetscan-vert). Details about predictive and validated transcripts were obtained by searching hsamiR-455-3p.1 and hsa-miR-455-3p.2 isoforms. Target genes were checked for following parameters: (i) their representative transcripts, (ii) number of 3P-seq tags supporting UTR+5, (iii) link to sites in UTRs, (iv) conserved sites/poorly conserved sites, (v) cumulative weighted context++ score, (vi) total context++ score, and (vii) aggregate PCT (preferentially conserved targeting) values. Further, predicted consequential pairing showed the miRNA-target complementarity at inside or outside the seed regions of miRNAs was checked at untranslated regions links (http://www.targetscan.org).

Statistical Analysis. The real-time RT-PCR data was analyzed by using the formula 2-ΔCT value of genes in each sample from AD patient's samples and controls. Statistical analysis was performed with Prism software, v, 6 (La Zolla, Calif.). P-value was calculated, based on the unpaired t-tests for analyzing two groups and using one way comparative analysis of variance (ANOVA) when comparing between more than two groups. ROC curve was plotted based on the 1CT value of samples in patients and control groups. Correlation analysis was performed using two tailed Pearson correlation coefficient (r) calculation considering 95% confidence interval. P<0.05 was considered statistically significant.

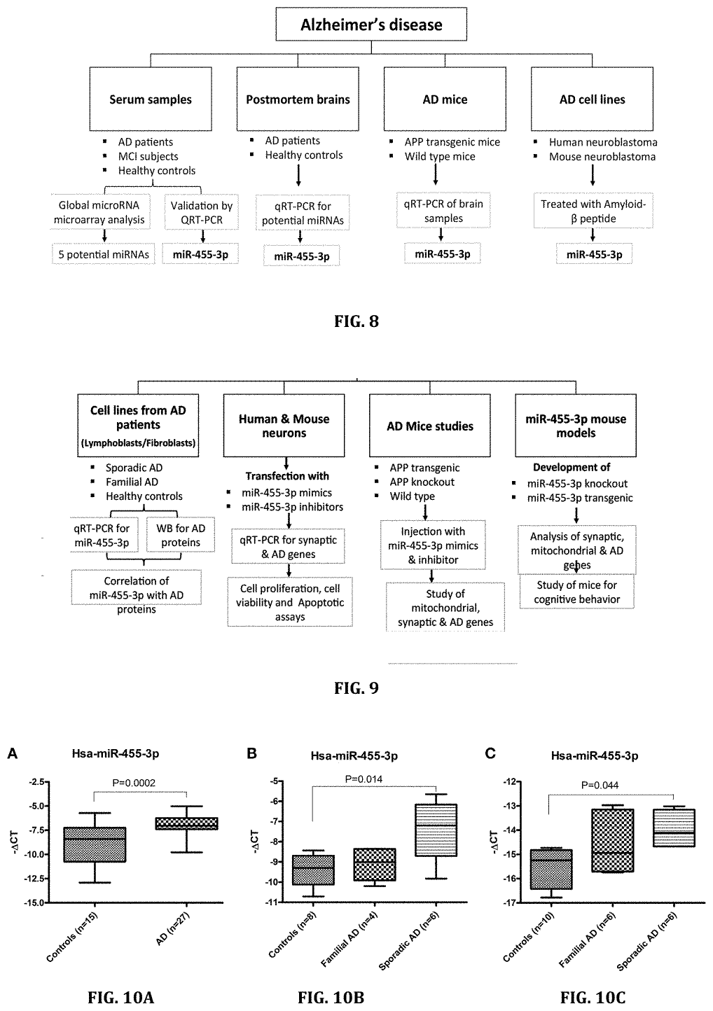

Up Regulation of miR-455-3p Expression in AD. AD Postmortem Brains—Total RNA was extracted from the postmortem brains of healthy controls (n=15) and AD patients (n=27) and expression of hsa-miR-455-3p was quantified by real-time RT-PCR analysis. Fold-change was calculated based on the 1CT value of miR-455-3p in AD patients' vs. healthy controls. The (ΔCT) value (mean±SD) was significantly (P=0.0001) higher in AD patients (−6.89±0.21) compared to the healthy controls (−8.94±0.56; FIG. 10A). Interestingly, fold-change analysis indicated the significantly higher expression of miR-455-3p in AD patients.

AD Fibroblasts—Similarly, expression of miR-455-3p was quantified in the skin fibroblast cells generated form familial AD patients (n=4), sporadic AD patients (n=6), and healthy control subjects (n=8). Differences in ΔCT values was evaluated among three groups using one-way ANOVA. Results showed the higher (−ΔCT) values (mean±SD) of miR-455-3p in familial and sporadic patients compared to controls. However, significant difference (P=0.014) in (−ΔCT) value was observed in sporadic cases (−7.35±1.39) compared to control samples (−9.37±0.76; FIG. 10B). AD B-Lymphocytes—Further, the inventors checked the level of miR-455-3p in B-lymphocytes obtained from familial AD patients (n=6), sporadic AD patients (n=6), and healthy controls (n=10). The (−ΔCT) (mean±SD) value was compared among three group using one-way ANOVA. Analysis showed the variations in miR-455-3p level among these groups, however significant difference (P=0.044) in (−ΔCT) value was reported between sporadic AD cases (−13.98±0.73) and controls (−15.50±0.80; FIG. 10C). Hence, results obtained from AD postmortem brains, AD fibroblast, and AD B-lymphocyte were conclusively confirmed the decisive role of miR-455-3p in AD assessment.

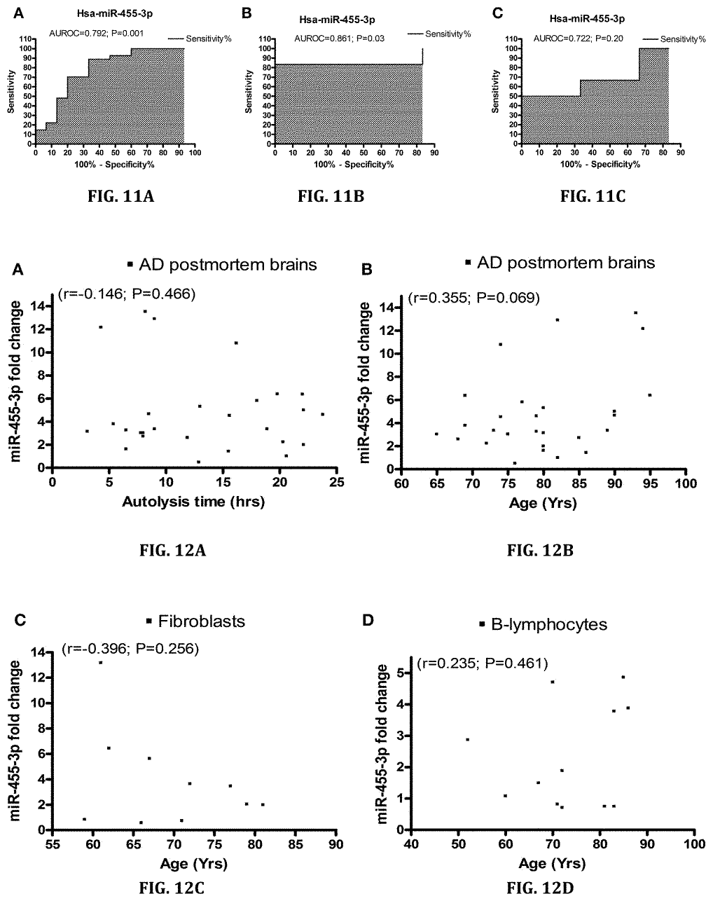

Receiver Operating Characteristics Curve Analysis of miR-455-3p. AD Postmortem Brains—To determine the diagnostic performance of miR-455-3p expression in AD patients, ROC curve was plotted using (ΔCT) values of miR-455-3p in AD patients and healthy controls. Analysis showed the significant area under ROC curve (AUROC) value of miR-455-3p (AUROC=0.792) with the 95% confidence interval was 0.637-0.948 (P=0.0018). The cut-off value was 8.16 with sensitivity of 88.89% (95% confidence interval: 70.84-97.65%) and specificity was 66.67% (95% confidence interval: 38.38-88.18%) in AD brain samples compared with healthy controls (FIG. 11A).

In AD Fibroblasts—ROC curve was analyzed for miR-455-3p expression in fibroblast cells form familial and sporadic AD patients vs. healthy controls. However, significant AUC value was obtained for ROC curve when comparing between sporadic AD patients with healthy controls. AUROC value was (0.861) with 95% confidence interval of 0.6036-1.119 (P=0.037). The cut-off value was 9.12 with sensitivity of 83.33% (95% confidence interval: 35.88-99.58%) and specificity was also 66.67% with confidence interval (22.28-95.67%; FIG. 11B).

In AD B-Lymphocytes—Similarly ROC curve for miR-455-3p was analyzed in B-lymphocytes of AD patients. Analysis between sporadic AD patients and healthy controls showed the fair AUROC value (0.722) with 95% confidence interval of 0.4185-1.026 (P=0.20). The cut-off value was 14.90 with sensitivity of 66.67% (95% confidence interval: 22.28-95.67%) and specificity was 50.00% (95% confidence interval: 11.81-88.19%; FIG. 11C). Thus, analysis showed that ROC analysis of miR-455-3p in B-lymphocytes was not significant. However, data from postmortem AD brains and AD fibroblasts cells showed significant ROC curve data further confirmed that the miR-455-3p as a valuable molecule capable of discriminating the patients with AD from healthy individuals.

Correlation of miR-455-3p Expression with Patients' Demographic Data. The inventors analyzed miR-455-3p expression levels in relation to (1) postmortem interval, (2) AD patients' age, and also (3) donors' age of fibroblasts, and (4) B-lymphocytes using Pearson correlation coefficients (r). AD postmortem brains showed a negative correlation r=−0.146 (with 95% confidence interval: −0.498 to 0.247; P=0.466) between brains postmortem interval and miR-455-3p expression level (FIG. 12A). Whereas a positive correlation r=0.355 (with 95% confidence interval: −0.029 to 0.647; P=0.069) was observed between the age of AD postmortem brains and miR-455-3p level (FIG. 12B). However, P-values were not significant in both cases. Thus, results showed a trend of reduced levels of miR-455-3p with increased postmortem interval and increased trend of miR-455-3p with patients' age. As shown in FIG. 12C, FIG. 12D, donors' age for fibroblasts (r=−0.396, 95% confidence interval: −0.821 to 0.310; P=0.256), and B-lymphocytes (r=0.235, 95% confidence interval: −0.391 to 0.713; P=0.461), the inventors did not find statistical significance, between donors age with miR-455-3p levels for fibroblasts and B-lymphocytes, indicating that donors age do not affect miR-455-3p expression levels.

In-Silico Analysis for miR-455-3p Function in AD. In-silico analysis was performed to understand the functions of miR-455-3p and its possible role in AD pathogenesis. Analysis was performed using the various bio-informatics algorithms such as DIANA-MICROT, MICRORNA.ORG, MIRDB, RNA22-HAS, TARGETMINER, and TARGETSCAN-VERT. As per miRbase database, a total of 3,102 reads of miR-455-3p has been detected by deep sequencing in 62 experiments (www.mirbase.org). Each algorithm was run for miR-455-3p and validated/predictive target genes were analyzed. A total of 323 predicted transcripts/human genes were identified with conserved miR-455-3p.2 binding site. Out of these genes most potential 13 targets genes were screened for those were having the roles in AD pathogenesis. Important ones were: APP, NGF, USP25, PDRG1, SMAD4, UBQLN1, SMAD2, TP73, VAMP2, HSPBAP1, and NRXN1 (Table 7). miR-455-3p having at least one or two binding site at 3′UTR of the genes and total context++ score ranges from −0.1 to −0.46. For e.g., miR-455-3p binds at the two sequence sites of 3′ UTR of APP gene at sequence position 522-528 and 3,139-3,145. Interaction of miR-455-3p at these sites will influence the expression level of APP genes. Hence, these analyses indicated the possible way the miR-455-3p involved in AD pathogenesis.

TABLE 7
Predictive/validated gene targets of miR-455-3p involved in AD

| | | Ortholog | | | | | Cumulative | Total | |
|---|---|---|---|---|---|---|---|---|---|
| | | of | | | | Conserved | weighted | context | |
| | Representa- | target | Representative | | 3P-seq | sites | context ++ | ++ | Aggregate |
| S. No | tive miRNA | gene | transcript | Gene name | tags + 5 | total | score | score | PCT |
| 1 | hsa-miR- | NGF | ENST00000369512.2 | nerve growth | 27 | 1 | −0.46 | −0.46 | 0.38 |
| | 455-3p.2 | | | factor (beta | | | | | |
| | | | | polypeptide) | | | | | |
| 2 | hsa-miR- | USP25 | ENST00000285681.2 | ubiquitin | 2012 | 2 | −0.45 | −0.45 | 0.6 |
| | 455-3p.2 | | | specific | | | | | |
| | | | | peptidase 25 | | | | | |
| 3 | hsa-miR- | PDRG1 | ENST00000202017.4 | p53 and DNA- | 116 | 1 | −0.45 | −0.45 | <0.1 |
| | 455-3p.2 | | | damage | | | | | |
| | | | | regulated 1 | | | | | |
| 4 | hsa-miR- | SMAD4 | ENST00000398417.2 | SMAD family | 403 | 2 | −0.3 | −0.32 | <0.1 |
| | 455-3p.2 | | | member 4 | | | | | |
| 5 | hsa-miR- | UBQLN1 | ENST00000376395.4 | ubiquilin 1 | 471 | 2 | −0.3 | −0.33 | <0.1 |
| | 455-3p.2 | | | | | | | | |
| 6 | hsa-miR- | APP | ENST00000346798.3 | amyloid beta | 4570 | 2 | −0.29 | −0.35 | <0.1 |
| | 455-3p.2 | | | (A4) precursor | | | | | |
| | | | | protein | | | | | |
| 7 | hsa-miR- | SMAD2 | ENST00000262160.6 | SMAD family | 1196 | 2 | −0.2 | −0.28 | 0.33 |
| | 455-3p.1 | | | member 2 | | | | | |
| 8 | hsa-miR- | TP73 | ENST00000378280.1 | tumor protein | 831 | 1 | −0.14 | −0.14 | 0.3 |
| | 455-3p.1 | | | p73 | | | | | |
| 9 | hsa-miR- | VAMP2 | ENST00000316509.6 | vesicle- | 1840 | 1 | −0.11 | −0.11 | 0.26 |
| | 455-3p.1 | | | associated | | | | | |
| | | | | membrane | | | | | |
| | | | | protein 2 | | | | | |
| | | | | (synaptobrevin | | | | | |
| | | | | 2) | | | | | |
| 10 | hsa-miR- | HSPBAP1 | ENST00000383659.1 | HSPB (heat | 22 | 1 | −0.11 | −0.15 | <0.1 |
| | 455-3p.1 | | | shock 27 kDa) | | | | | |
| | | | | associated | | | | | |
| | | | | protein 1 | | | | | |
| 11 | hsa-miR- | NRXN1 | ENST00000342183.5 | neurexin 1 | 5 | 1 | −0.1 | −0.1 | 0.3 |
| | 455-3p.1 | | | | | | | | |

The purpose of the study was to determine the blood based peripheral biomarkers for AD. The inventors recently conducted a high throughput microRNA analysis using serum-derived RNA samples from MCI subjects, AD patients, and healthy control subjects (Kumar et al., 2017). The inventors found several differentially expressed miRNAs in MCI subjects and patients with AD relative to healthy controls. Further, the inventors verified differentially expressed miRNAs using real-time RT-PCR from serum-derived miRNAs, and also from cell and mouse models of AD. In the current study, the inventors extended the investigations using large numbers of fibroblasts, B-lymphocytes from familial and sporadic AD patients and age-matched control subjects. The inventors found miR-455-3p levels were upregulated in the fibroblasts and B-lymphocytes from AD patients relative to healthy control subjects. However, a significant difference was observed in the cells form sporadic AD patients compared to healthy controls. Similarly, in B-lymphocytes, miR-455-3p level was significantly upregulated in sporadic AD cases compared to controls (P=0.044). Receiver operating characteristic curve analysis indicated the significant area under curve value of miR-455-3p in AD postmortem brains (AUROC=0.792; P=0.001) and AD fibroblasts cells (AUROC=0.861; P=0.03). These observations show that miR-455-3p is a biomarker for sporadic AD.

An early stage pre-clinical diagnostic biomarkers are urgently needed to detect disease process early on in life and take necessary action to prevent and/or delay disease progression. Recent molecular biology studies using serum/plasma revealed that several circulatory microRNAs can be used as potential peripheral biomarkers for AD (Kumar and Reddy, 2016). However, these circulatory microRNAs are needed further validation using postmortem AD brains and cell and mouse models of AD. Therefore, more accurate and mechanistic research is needed to determine potential candidates as biomarkers for AD. As mentioned above, the recent lab study on AD serum samples and other AD sources/AD mouse model unveiled the miR-455-3p as potential biomarker candidate for AD. Many other reports identified the role of miR-455-3p in several cancers and chondrogenic differentiation (Chen et al., 2016; Cheng et al., 2016; Li et al., 2016; Liu et al., 2016; Qin et al., 2016; Zheng et al., 2016; Zhao et al., 2017). The study was the first to reveal the higher expression level of miR-455-3p in persons with AD.

Current study is the continuation of the ongoing biomarkers research project in the Reddy Lab. Here, first the inventors investigated miR455-3p levels in the well-defined postmortem brain tissues from AD patients. All tissues were dissected from the affected area (Broadmann's area 10) of AD patients and commonly used for the investigation of AD pathogenesis (Wilcock et al., 2015; De Rossi et al., 2016; Shackleton et al., 2017). The current study on AD postmortem findings revealed that miR-455-3p levels are significantly increased in a large number (n=27) of AD brains and a significant AUC value also strengthen its biomarker potential. However, the inventors don't know the exact reason for the upregulation of miR-455-3p in AD brains and further, the inventors still do not know molecular mechanism(s) of its increased levels. By way of explanation, and in no way a limitation of the present invention, and eased on current findings, the upregulation of miR-455-3p—may be a compensatory to the amyloid beta toxicity in disease process.

Beside brain tissues, the inventors also investigated the AD fibroblasts and B-lymphocytes for miR-455-3p expression. These AD cell lines are the good sources for the investigation of AD pathologies and associated molecular changes in the patients' genome (Khan and Alkon, 2016). Both cell types showed the significantly higher levels of miR-455-3p, especially in sporadic AD cases but not in familial AD. Further, high level of miR-455-3p in AD fibroblasts and lymphoblasts indicate that increased levels of miR-455-3p is a typical feature of AD—both in the brain and peripheral cells. Alteration of miR-455-3p expression in AD cell lines indicates the strong molecular association of miR-455-3p with AD progression.

In order to expose the roles and functions of miR-455-3p in AD, in-silico analysis provides the valuable information. As described 11 genes were reported to involve in AD progression (FIGS. 3A-E) (Burton et al., 2002; Li et al., 2004; Slifer et al., 2006; Toth et al., 2013; Sindi et al., 2014; Jung et al., 2015, 2016; Malkki, 2015; Vallortigara et al., 2016; Kuruva et al., 2017). To understand the roles of miR-455-3p in AD, expression of these genes needs to be studied by using miR-455-3p modulation approaches (mimics/inhibitors). In this direction, next phase of the study is to determine the effect of miR-455-3p on its AD related target genes. Current focus of the laboratory is to understand the role of miR-455-3p in APP processing and amyloid beta modulation, using miR-455-3p mimics and inhibitor treatments. The inventors also predict that two potential binding sites of miR-455-3p at the 3′UTR of APP gene may be involved in the modulation of full length APP. Further, the inventors also predict that miR-455-3p affects the APP processing and amyloid beta production.

In summary, for the first time, the inventors report that microRNA455-3p is a peripheral biomarker for AD. These findings are based on (1) blood-based circulatory microRNAs from AD patients, (2) AD postmortem brains, AD cell lines, and AD mouse models and a large number of AD fibroblasts and lymphoblasts.

### Example 3

The purpose of this example was to understand the protective role of miR-455-3p against abnormal amyloid precursor protein (APP) processing, amyloid beta (Aβ) formation, defective mitochondrial biogenesis/dynamics and synaptic damage in AD progression. In-silico analysis of miR-455-3p has identified the APP gene as a putative target. Using mutant APP cells, miR-455-3p construct, biochemical and molecular assays, immunofluorescence and transmission electron microscopy (TEM) analyses, the inventors studied the protective effects of miR-455-3p on—1) APP regulation, amyloid beta (Aβ)(1-40) & (1-42) levels, mitochondrial biogenesis & dynamics; 3) synaptic activities and 4) cell viability & apoptosis. A luciferase reporter assay confirmed the binding of miR-455-3p at the 3′UTR of APP gene. Immunoblot, sandwich ELISA and immunostaining analyses revealed that the reduced levels of the mutant APP, Aβ(1-40) and Aβ(1-42), and C99 by miR-455-3p. It was also found the reduced levels of mRNA and proteins of mitochondrial biogenesis (PGC1α, NRF1, NRF2, and TFAM) and synaptic genes (synaptophysin and PSD95) in mutant APP cells; on the other hand, mutant APP cells that express miR-455-3p showed increased mRNA and protein levels of biogenesis and synaptic genes. Additionally, expression of mitochondrial fission proteins (DRP1 and FIS1) were decreased while the fusion proteins (OPA1, Mfn1 and Mfn2) were increased by miR-455-3p. TEM analysis showed a decrease in mitochondria number and an increase in the size of mitochondrial length in mutant APP cells transfected with miR-455-3p. Based on these observations, it is demonstrated here that miR-455-3p regulates APP processing and is protective against mutant APP-induced mitochondrial and synaptic abnormalities in AD.

The inventors identified several potential genes that are targeted by miR-455-3p and very important in AD pathogenesis. APP gene was one of the top-targeted genes in the miRNA analysis. The precise molecular links between miR-455-3p and APP gene are now described, particularly whether upregulated miR-455-3p is protective or deleterious in the progression and pathogenesis of AD and 2) further, the impact of 455-3p on APP processing & Aβ production, mitochondrial biogenesis, mitochondrial dynamics and synaptic activity in AD neurons is unclear. To address these questions, APP processing, mitochondrial biogenesis, mitochondrial dynamics, mitochondrial morphology, synaptic activity and cell viability in mutant APP cells that express miR-455-3p was studied.

Cell culture. The N2a mouse neuroblastoma cell line was purchased from the ATCC (American Type Culture Collection) and was maintained in the Reddy laboratory. The cells were grown in DMEM+F12 (1:1) supplemented with 10% FBS at 37° C. with 5% CO₂.

Mutant APP cDNA construct. The mutant APP Swe cDNA clone (pCAX-APP Swe/Ind) was obtain from the laboratory of Dr. Arubala Reddy [32]. APP Swe cDNA was cloned into a mammalian expression vector pRP-euro-CAG. The pRP vector is a puc backbone having a CMV promoter and an SV40 polyadenylation site with puromycin selection for stable transfection. The sequence output was confirm with the NCBI sequence hAPP [NM 201414.2]*(K595N M596L V642F), relevant sequence incorporated herein by reference.

MiR-455-3p expression vector. MiR-455-3p expression vector (pRP[Exp]-U6>hsa-miR-455-3p-CAG-EGFP) was purchased from VectorBuilder (Cyagen Biosciences, Santa Clara, Calif., USA). It was a 5128 bp plasmid propagated in Stb13 host and contained a Green Fluorescent Protein (GFP) encoding gene (data not shown). GFP expression readily allowed the detection and confirmation of transfection.

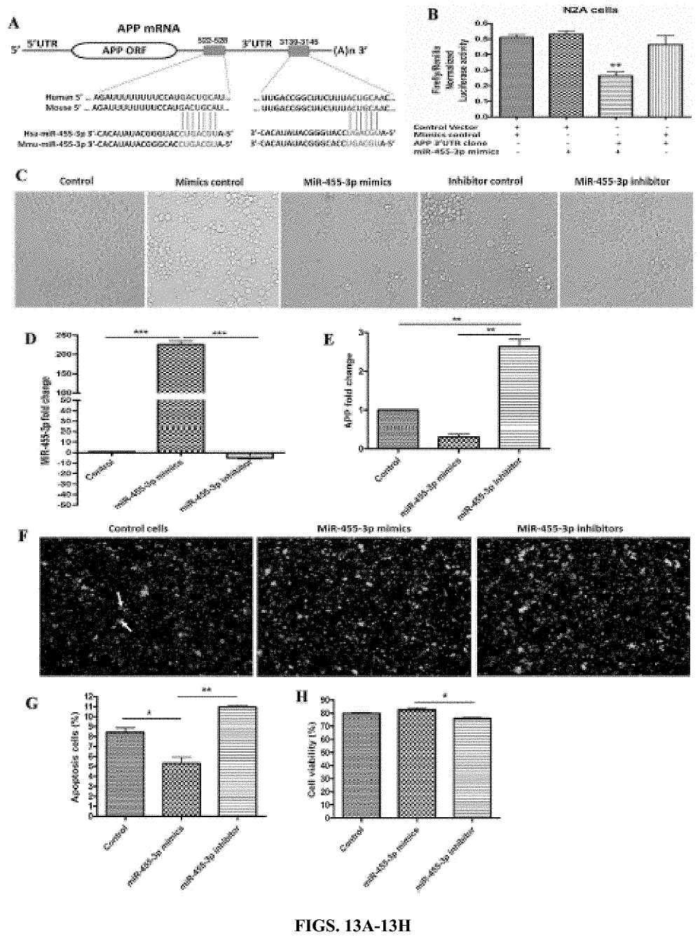

Target prediction analysis of miR-455-3p. In silico analysis was performed with various bio-informatics algorithms, such as DIANA-MICROT, MICRORNA.ORG, MIRDB, RNA22-HAS, TARGETMINER, and TARGETSCAN-VERT. MiR-455-3p had at least 2 binding sites at 3′UTR of the genes and total context++ score ranges from −0.1 to −0.46. For example, miR-455-3p binds at the 2 sequence sites of 3′ UTR of the APP gene at sequence positions 522-528 and 3,139-3,145 (FIG. 13A).

FIGS. 13A to 13H—MiR-455-3p interaction with the APP gene and its effects on neuroblastoma cells survival. (FIG. 13A) The two putative binding sites of miR-455-3p at the 3′-UTR regions of a wild-type APP gene in humans and mice. Region A (522-528) had 7 nucleotide binding sites, and Region B (3139-3145) had 6 nucleotide binding sites with the seed sequence of miR-455-3p in both the human and mouse wild-type APP gene (shown in green). (FIG. 13B) Luciferase reporter assay for miR-455-3p binding with the APP gene. Normalized luciferase activity (Firefly/Renilla) in neuroblastoma cells co-transfected with the APP 3′UTR clone (HmiT009578-MT06) and miR-455-3p mimics. Firefly/Renilla luciferase activity of the APP 3′UTR clone was significantly decrease by the miR-455-3p. (FIG. 13C) Representative images of neuroblastoma cells morphology after 24 hr of miR-455-3p mimics and inhibitor transfection (10× magnification). Quantitative measurement of miR-455-3p (FIG. 13D), and APP fold-change (FIG. 13E), in cells at 24 hr post mimics and inhibitor transfection. (13F) Representative images of Annexin V and PI staining of cells at 24 hr post-transfection of miR-455-3p mimics and inhibitor. White arrow represents the viable (green) and dead (red) cells. Percentage (%) of apoptotic cells (FIG. 13G), and viable cells populations (FIG. 13H), after 24 hr of miR-455-3p mimics and inhibitor transfection. (*P<0.05) (**P<0.01) (***P<0.001)

Luciferase reporter assay. To confirm the binding of miR-455-3p at the 3′UTR of the APP gene, neuroblastoma cells were plated at a density of 5×10⁴ cells per well in a 24-well plate for one day before transfection. The cells were then transfected with APP 3′UTR target expression clone (HmiT009578-MT06) for the human APP (NM 000484.2) and miRNA target clone control vector having mutated 3′UTR of APP gene pEZX-MT06 (CmiT000001-MT06) (Genecopoeia, Rockville, Md., USA). Cells were also transfected with miR-455-3p mimics and mimic controls (Applied Biological Materials, Inc., Richmond, BC V6V 2J5, Canada). After 24 hr of transfection, luciferase activity was measured with the Luc-Pair™ Duo-Luciferase Assay Kit 2.0 (Genecopoeia, Rockville, Md., USA) as per manufacturer instructions. The samples were read with an illuminometer.

Transfection of miR-455-3p mimics and inhibitors. Neuroblastoma cells were transfected with 25 nM (final concentration) of miR-455-3p mimic oligonucleotide and miR-455-3p inhibitor oligonucleotide (Applied Biological Materials, Richmond, BC, CANADA) using Lipofectamine™ 2000 reagent (Invitrogen Life Technologies, Carlsbad, Calif., USA) following the manufacturer's instructions.

Transfection of the MiR-455-3p vector and the mutant APP cDNA. Neuroblastoma cells were cultured in antibiotic-free media, in 6-well plates one day before transfection. On the next day, when cells reached 70% to 80% confluence, they were transfected with the miR-455-3p vector and the mutant APP cDNA with the Lipofectamine™ 2000 reagent. Mutant APP cDNA contains three identical sequences at three different locations those were complementary to the seed sequence of miR-455-3p.

Apoptotic assay. A cell-based apoptosis assay was performed, using the Cellometer Vision CBA Image Cytometry System (Nexcelom Bioscience, LLC, Lawrence, Mass., USA) with 2 fluorophores—Annexin V-FITC and propidium iodide (PI) staining solutions, following manufacturer's instructions. Briefly, neuroblastoma cells were harvested using trypsin, then spun down to 300 g for 3 min. The pellets were washed with 1×PBS. Cells were counted using a hematocytometer, and 100,000 to 150,000 cells were collected and resuspended in 40 μl of an Annexin V binding buffer. Five μl of Annexin V-FITC reagent (green) and PI (red) each were added to the binding buffer containing the cells. The solution was gently mixed by pipetting up and down. It was then incubated for 15 min at room temperature in the dark. After incubation, 250 μl of 1×PBS was added and spun down at 300 g for 3 min. Cell pellets were re-suspend in 50 μl of an Annexin V binding buffer and assessed for apoptosis analysis [32].

Cell viability test (MTT assay). Mitochondrial respiration, an indicator of cell viability, was assessed in the neuroblastoma cells from control and experimental treatments (n=4), using the mitochondrial-dependent reduction of 3-(4,5-dim ethyl-thiazol-2-yl)-2,5-diphenyl-tetrazolium bromide (MTT) to formazan as described in Reddy et al, (2016) [33]. Cells were trypsinized until they have lifted off the cell culture plate, then spin down at 300 g for three minutes, decant the supernatant and re-suspend cells in 1×PBS. 20μl of cells were transferred to the countering chamber, cell viability was determined by using the Cellometer Vision CBA Image Cytometry System.

Assessment of miR-455-3p levels—Quantification of miR-455-3p involved 3 steps:

(i) Polyadenylation. One μg of total RNA was polyadenylated with a miRNA First-Strand cDNA synthesis kit (Agilent Technologies Inc., CA, USA), following manufacturer's instructions [18].

(ii) cDNA synthesis. Ten μL of polyadenylated miRNAs were processed for cDNA synthesis with the miRNA First-Strand cDNA synthesis kit (Agilent Technologies Inc., CA, USA). Resulting cDNAs were diluted with 20 μL of RNase-free water and stored at −80° C. for further analysis.

(iii) Real-time qRT-PCR. Real-time RT-PCR was performed by preparing a reaction mixture containing 1 μL of miRNA-specific forward primer (10 μm), 1 μL of a universal reverse primer (3.125 μM) (Agilent Technologies Inc., CA, USA), 10 μl of 2×SYBR Green PCR master mix (Applied Biosystems CA), and 1 μL of cDNA. To this mixture RNase-free water was added up to a 20-μL final volume. Primers used in the current study were synthesized commercially (Integrated DNA Technologies, Inc., City, Iowa, USA). To normalize the miRNA expression, snoRNA-202 (small nuclear RNA) expression was also quantified in the cells, which was used as an internal control. The reaction mixture for each sample prepared in triplicates was set in the 7900HT Fast Real-Time PCR System (Applied Biosystems). MiRNA fold change were calculated by using the formula (2^{-ΔΔct}) [18, 21].

Messenger RNA levels of APP, mitochondrial biogenesis and synaptic genes. Quantification of mRNA levels in APP, mitochondrial biogenesis, and synaptic genes was carried out with real-time RT-PCR using methods described in Reddy et al, (2016) [33]. The oligonucleotide primers were designed with primer express software (Applied Biosystems) for the housekeeping genes β-actin, APP, PGC1α, NRF1, NRF2, TFAM, DRP1, FIS1, OPA1, Mfn1, Mfn2, synaptophysin, and PSD95. SYBR-Green chemistry-based quantitative real-time RT-PCR was used to measure mRNA expression of these genes using β-actin and GAPDH as housekeeping genes, as described by Manczak et al, (2016) [34].

Immunoblot analysis for the mutant APP protein. Immunoblot analysis was performed, using protein lysates prepared from all groups of cells (cells transfected and untransfected with mutant APP, miR455-3p), using the 6E10 antibody that recognizes full-length mutant human APP and Aβ. The inventors also performed immunoblot analysis for all mitochondrial biogenesis proteins. Twenty μg of protein lysates were resolved on a 4-12% Nu-PAGE gel (Invitrogen). The resolved proteins were transferred to nylon membranes (Novax Inc., San Diego, Calif., USA) and then incubated for 1 hr at room temperature with a blocking buffer (5% dry milk dissolved in a TBST buffer). The nylon membranes were incubated overnight with the primary antibodies. The membranes were washed with a TBST buffer 3 times at 10-min intervals and then incubated for 2 hr with an appropriate secondary antibody, sheep anti-mouse HRP 1:10,000, followed by three additional washes at 10-min intervals. Proteins were detected with chemiluminescence reagents (Pierce Biotechnology, Rockford, Ill., USA), and the bands from the immunoblots were visualized [32].

Immunostaining and immunofluorescence analysis. To determine the immunoreactivity and intensity of the APP protein, the inventors performed immunofluorescence analysis using neuroblastoma cells transfected with a miR-control vector, a miR-455-3p vector, and a mutant APP cDNA. The cells were co-transfected with an APP clone and a miR-455-3p vector. The cells were grown on coverslips for 24 hr post-transfection. They were then were washed with 1×PBS three times for 5 min each and fixed in freshly prepared 4% paraformaldehyde in PBS for 10 min. The cells were washed again with PBS and permeabilized with 0.1% Triton-X100 in PBS. The cells were blocked with a 1% blocking solution (Invitrogen) for 1 hr at room temperature. The cells were incubated with 6E10 primary antibody (1:200 dilution) (Biolegend, San Diego, Calif.) overnight at 4° C. After incubation, the cells were washed 3 times with PBS, for 10 min each. The cells were incubated with an anti-mouse secondary antibody conjugated with Fluor 488 (1:500 dilution) (Invitrogen) for 1 hr at room temperature, then washed 3 times with PBS, and mounted on slides. Photographs were taken with a multiphoton laser scanning microscope system (ZeissMeta LSM510). To quantify the immunoreactivities of antibodies, 10-15 photographs were taken at 20× magnification. Statistical significance was assessed by the intensities of red, green or blue, using NIH ImageJ software [32].

ELISA for Aβ₍₁₋₄₀₎ and Aβ₍₁₋₄₂₎. The protein lysates were prepared from neuroblastoma cells pellets and soluble Aβ levels were measured, using human-specific Aβ(1-40) and Aβ(1-42) sandwich ELISA kit. The inventors used Human Quantikine® ELISA Amyloid β (aa1-40) and (aa1-42) immunoassay kit as per manufacturer instructions (R&D Systems, Inc. Minneapolis, Minn., USA).

Transmission electron microscopy. Experiments were performed in 60-mm Petri dishes. Neuroblastoma cells were transfected with a miR-control vector, the miR-455-3p vector, and the mutant APP cDNA; and were co-transfected with the APP clone and the miR-455-3p vector. After 48 hr, the cells were washed with 5 ml 1×PBS. Cell pellets were dissolved in a fixative solution (8% glutaraldehyde, 16% paraformaldehyde, and 0.2M sodium cacodylate buffer) for 1 hr at room temperature. The cells were removed from the fixative solution and scraped into one ml of a fresh fixative solution. Cells were incubated at room temperature for 30 min. The cells were centrifuge at 300 g for 3 min [32]. The resulting cell pellet was undergone electron microscopy at the Imaging Core Facility at Texas Tech University.

Statistical considerations. Statistical analyses were conducted, by using the student T-test for analyzing 2 groups of samples, and one-way comparative analysis of variance (ANOVA) was used for analyzing more than two groups of samples. Significant differences in five group of samples were calculated by Bonferroni's multiple comparison tests. Statistical parameters were calculated, using Prism software, v6 (La Zolla, Calif., USA). P<0.05 was considered statistically significant.

MiR-455-3p interaction with the APP gene. The inventors in silico miRNA target prediction analysis showed two binding sites of miR-455-3p at 3′ UTR of the APP gene (ENST00000346798.3) at sequence positions 522-528 and 3,139-3,145 (FIG. 13A). The cumulative weighted context++ score was −0.29, total context++ score was −0.35, and the aggregate PCT was <0.1 for miR-455-3p (Table 8). The miR-455-3p binding sequence at the 3′UTR of APP mRNA was similar in the human and mouse genome. To confirm these results, a luciferase reporter assay was performed on a vector having a wild-type APP 3′UTR sequence and on a control vector. Luciferase activity was significantly (P=0.002) reduced in the cells transfected with miR-455-3p mimics compared to their respective controls (FIG. 13B). These results indicate that the 3′UTR site of mRNA from the APP protein consists of conserved binding sites for miR-455-3p.

TABLE 8
Parameters for miR-455-3p binding at 3'UTR of APP gene

| Previous TargetScan publication(s) | None |
|---|---|
| Aggregate PCT | <0.1 |
| Total context++ score | −0.35 |
| Cumulative weighted context++ score | −0.29 |
| Representative miRNA | hsa-miR-455-3p.2 |
| 6mer sites | 1 |
| Poorly conserved 7mer-A1 sites | 1 |
| Poorly conserved 7mer-m8 sites | 1 |
| Poorly conserved sites total | 2 |
| 3P-seq tags + 5 | 4570 |
| Gene name | amyloid beta (A4) precursor protein |
| Representative transcript | ENST00000346798.3 |
| Target gene | APP |

MiR-455-3p functional analysis. (a) Neuroblastoma cells morphology treated with miR-455-3p mimics and inhibitors. Since miR-455-3p target APP gene regulation, the inventors sought to determine the effects of increased and decreased levels of miR-455-3p on cell morphology. The inventors analyzed the cell morphology after miR-455-3p mimics and inhibitor treatment. Microscopic examination of cells at 24 hr post-transfection with the miR-455-3p mimics showed extended cell proliferation compared to the controls (FIG. 13C). The cells transfected with miR-455-3p inhibitors did not show normal and healthy proliferation. These findings suggest that higher expression levels of miR-455-3p mimics promoted cell growth, on the other hand, inhibitors reduced cell proliferation.

(b) MiR-455-3p and APP expression analysis. To confirm the regulation of APP gene by miR-455-3p, cells were transfected with miR-455-3p mimics and inhibitors, followed by the quantification of miR-455-3p and APP mRNA expression at 24 hr post-transfection. qRT-PCR analysis showed the significant upregulation of miR-455-3p (225-fold) (P=0.0001) in cells transfected with miR-455-3p mimics. On the contrary, cells treated with miR-455-3p inhibitors showed a significant downregulation (−5.23-fold) of miR-455-3p relative to untreated cells (FIG. 13D).

In the qRT-PCR analysis, it was found that mRNA levels of APP were reduced by 0.3-fold (P=0.001) in the cells transfected with the miR-455-3p mimics. On the other hand, it was found 2.6-fold upregulation of APP expression in the cells transfected with the miR-455-3p inhibitors (FIG. 13E). These results agree with the luciferase assay data that miR-455-3p had the potential to modulate the APP expression.

(c) Effects of miR-455-3p mimics and inhibitors on cell viability. As shown in FIGS. 13F and 13G, apoptotic cell death was reduced by 5.31% (P=0.042) in the cells transfected with miR-455-3p mimics relative to untransfected cells. On the contrary, apoptotic cell death was increased by 10.96% (P=0.0071) in cells transfected with miR-455-3p inhibitors. These findings point to the protective effects of miR-455-3p mimics on cell survival.

Cell viability analysis showed the increased levels of cell survival in cells transfected with miR-455-3p mimics compared to the untransfected cells (FIG. 13H). As expected, cell survival was significantly reduced in cells transfected with miR-455-3p inhibitors. These findings suggest that miR-455-3p mimics increases cell survival.

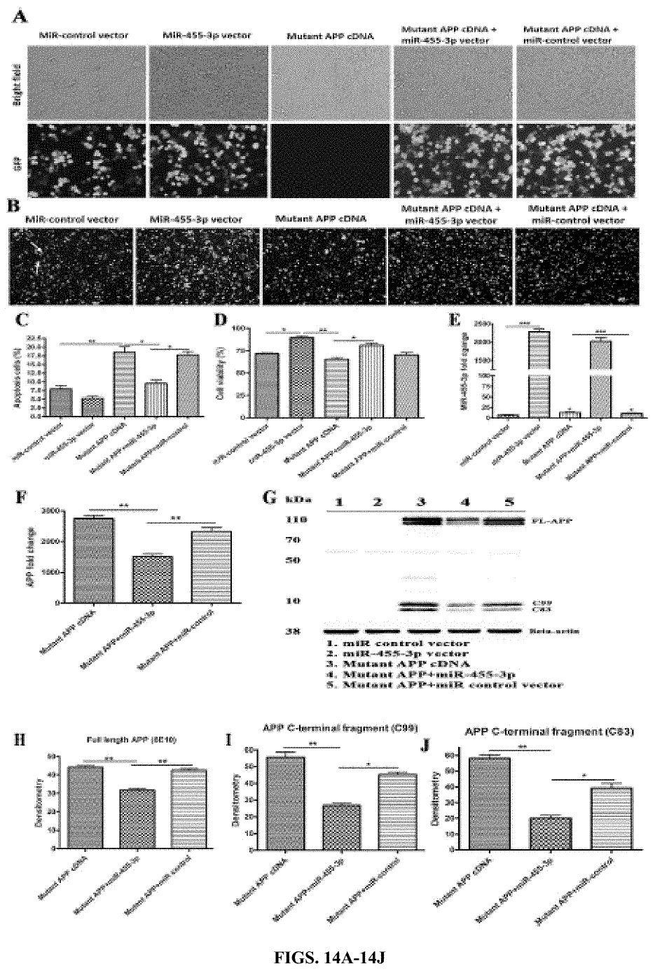

Effect of miR-455-3p on mutant APP. To determine the effect of mutant APP and miR-455-3p on cell survival and cell viability, cells were transfected with mutant APP cDNA and miR-455-3p vector. Cells were observed under a light microscope after 24 hours transfection with mutant APP and miR-455-3p individually and in combination. MiR-455-3p vector had a green fluorescence protein. As shown in FIG. 14A, cells transfected with miR-455-3p vector showed an increased number of cells relative to the miR-control vector (vector without miR-455-3p sequence) and mutant APP cDNA transfected cells.

FIG. 14A to 14J—Regulation of mutant APP cDNA expression by miR-455-3p. (FIG. 14A) Representative images for transfection of the miR-455-3p expression vector (GFP), the miR-control (GFP) vector, and the mutant APP cDNA in neuroblastoma cells (10× magnification). Bright field images and green fluorescence detected in the same field at 24 hr post-transfection (10× magnification). (FIG. 14B) Representative images of Annexin V and Propidium Iodide (PI) stained neuroblastoma cells transfected with the miR-control vector, the miR-455-3p vector, and the mutant APP cDNA. The white arrow indicates the populations of viable (green) cells and dead (red) cells. (FIG. 14C) Percentage (%) of an apoptotic cell population after 24 hr of the miR-455-3p vector, the miR-control vector, and the mutant APP cDNA transfecting cells. (FIG. 14D) Percentage (%) of a viable cell population after 24 hr of the miR-control vector, the miR-455-3p vector and the mutant APP cDNA transfecting cells. (FIG. 14E) Quantitative measurement of miR-455-3p fold change expression after 24 hr of the miR-455-3p vector and the mutant APP cDNA transfecting cells. (FIG. 14F) Quantitative measurement of APP mRNA folds change expression after 24 hr of the miR-455-3p vector and the mutant APP cDNA transfecting cells. (FIG. 14G) Western blot for APP (6E10), the C-terminal fragment of APP (C99) and B-actin proteins in cells transfected with the miR-control vector, the miR-455-3p vector, mutant APP cDNA and co-transfected cells. Quantitative measurement of (FIG. 14H) APP (6E10) and C-terminal fragments of APP C99 (FIG. 14I), and C83 (FIG. 14J) proteins levels by densitometry in the mutant APP cDNA and the miR-455-3p vector transfecting cells. (*P<0.05) (**P<0.01) (***P<0.001)

Significantly increased apoptotic cell death was observed in mutant APP cells relative to untransfected cells (18.46%) (P=0.001). Co-transfected cells with both mutant APP+miR-455-3p showed significantly reduced apoptotic cell death (9.54%) (P=0.031) relative to mutant APP cells (FIGS. 14B and 14C). Cell viability was significantly increased (91.35%) (P=0.014) in cells transfected with miR-455-3p compared to miR-control vector-transfected cells (71.95%) (FIG. 14D). Similarly, cell viability was higher in cells co-transfected with the mutant APP and miR-455-3p vector compared to cells transfected with mutant APP alone. These findings show that miR-455-3p is protective against mutant APP toxicity.

Effect of miR-455-3p on mRNA and protein levels of mutant APP. Next, the inventors determined the expression of miR-455-3p in miR-control vector, miR-455-3p vector and mutant APP transfected cells and how overexpression of miR-455-3p modulates the expression of mutant APP cDNA.

The miR-455-3p analysis revealed that increased expression levels of miR-455-3p (2285-fold) (P=0.0001) in cells transfected with miR-455-3p vector compared to the miR-control vector (FIG. 14E). The inventors also observed increased levels of miR-455-3p in cells transfected with mutant APP cDNA compared to miR-control vector (FIG. 14E). As shown in FIG. 14F, significantly reduced levels of mutant APP mRNA (1508-fold) (P=0.008) were found in cells co-transfected with miR-455-3p and mutant APP relative to cells transfected with mutant APP alone.

The immunoblot analysis findings agrees with mRNA data that significantly reduced the level of full-length APP and both C99 and C83 fragments in cells co-transfected with the miR-455-3p and mutant APP cells (P=0.0021) relative to mutant APP cells alone (FIGS. 14G, 14H, 14I and 14J). These observations suggest that miR-455-3p not only modulate full-length APP but also involved in c-terminal fragments of APP.

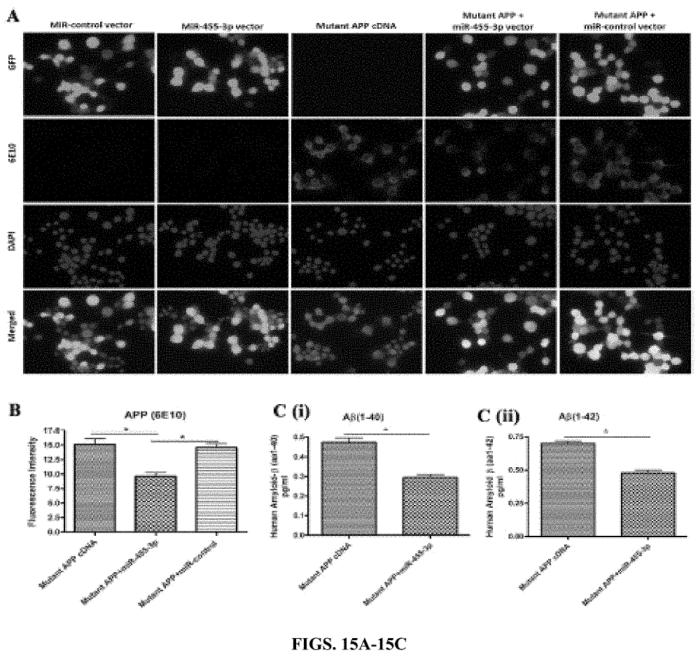

Effect of miR-455-3p on the localization of mutant APP. To determine the location and immunoreactivity levels of mutant APP and Aβ in cells transfected with miR-455-3p, mutant APP and in combination, immunostaining of cells were performed. As shown in FIG. 15A, cells transfected with miR-455-3p, showed green fluorescence protein. Expression intensity of APP (6E10, red) was significantly reduced in cells co-transfected with the miR-455-3p and mutant APP cDNA (P=0.027) compared to the cells transfected with mutant APP alone (FIGS. 15A and 15B). These findings further confirmed that miR-455-3p has a role in regulating mutant APP and Aβ levels.

FIGS. 15A to 15C—MiR-455-3p reduces APP and amyloid proteins. (FIG. 15A) Representative immunostaining images of neuroblastoma cells transfected with the miR-control vector, the miR-455-3p vector, the mutant APP cDNA, the mutant APP and miR-455-3p co-transfected. Cells were stain with the APP (6E10) antibody producing red fluorescence (20× magnification). Green fluorescence showed the miR-control and miR-455-3p vector expression and the nucleus of the cell stained with DAPI (blue). Fluorescence intensity (red) of the mutant APP was reduce in mutant APP and miR-455-3p co-transfected cells. (FIG. 15B) Quantitative measurement of fluorescence intensity of the APP (6E10) protein in mutant APP, miR-455-3p, and miR-control vector transfected cells. (FIG. 15C) ELISA analysis for amyloid ₍₁₋₄₀₎ and ₍₁₋₄₂₎ levels in neuroblastoma cells. Quantitative detection of the (i) human amyloid-β₍₁₋₄₀₎ and (ii) amyloid-β₍₁₋₄₂₎ peptide level in mutant APP cDNA and miR-455-3p vector transfected cells. (*P<0.05).

Effect of miR-455-3p on Aβ(1-40) and Aβ(1-42) levels. Since, miR-455-3p reduced the levels of full-length mutant APP, the levels of soluble Aβ₍₁₋₄₀₎ and Aβ₍₁₋₄₂₎ in neuroblastoma cells lysate were checked. Level of Aβ₍₁₋₄₀₎ was significantly reduced (P=0.011) in the cells co-transfected with the miR-455-3p and mutant APP compared to cells transfected with mutant APP alone (FIG. 15C). Similarly, the level of Aβ₍₁₋₄₂₎ was also significantly lowered by miR-455-3p in co-transfected cells with miR-455-3p and mutant APP compared to cells transfected with mutant APP alone (FIG. 15C). These observations suggest that miR-455-3p reduced both Aβ₍₁₋₄₀₎ and Aβ₍₁₋₄₂₎ levels in mutant APP cells.

Effect of miR-455-3p on mitochondrial biogenesis and synaptic activity. To determine the effect of miR-455-3p on mitochondrial biogenesis and synaptic proteins, the inventors assessed mitochondrial biogenesis (PGC1α, NRF1, NRF1, TFAM), and synaptic (synaptophysin, and PSD95) genes at mRNA and protein levels.

As shown in Table 9, mRNA levels of mitochondrial biogenesis genes PGC1α, NRF1, NRF1, TFAM were significantly reduced in the mutant APP cells relative to miR-control transfected cells. On the other hand, mRNA levels of mitochondrial biogenesis genes were significantly increase in co-transfected cells with miR-455-3p and mutant APP relative to cells transfected with mutant APP alone (Table 9).

TABLE 9
qRT-PCR analysis of neuroblastoma cells transfected with miR-control vector,
miR-455-3p vector, mutant APP cDNA and their mRNA fold change calculations relative to miR-
control vector

| | | (Fold change-mRNA/β-actin) | | | |
|---|---|---|---|---|---|
| | | | Mutant | Mutant | Mutant |
| | | miR-455- | APP | APP + miR- | APP + miR- |
| | Genes | 3p vector | cDNA | 455-3p | control |
| Mitochondrial biogenesis genes | PGC1α | 4.24** | −1.8* | 3.64** | −1.7* |
| | NRF1 | 3.18** | −1.4 | 2.48* | −4.81** |
| | NRF2 | 3.2* | −1.59* | 2.43* | −1.25* |
| | TFAM | 2.6* | −1.4* | 2.68* | −4.49** |
| Synaptic genes | Synaptophysin | 2.5* | −2.44* | 2* | −1.45* |
| | PSD95 | 3.25* | −3.29** | 2.78* | −2.37** |
| Mitochondrial fission genes | DRP1 | −1.42* | 2.36* | −1.18 | 2.43* |
| | FIS1 | −1.37* | 1.89* | −1.17 | 1.61* |
| Mitochondrial fusion genes | OPA1 | 1.59* | −1.93* | 1.85* | −1.4* |
| | Mfn1 | 1.92* | −1.46* | 1.48* | −1.76* |
| | Mfn2 | 2.01* | −1.14 | 1.93* | −1.19 |

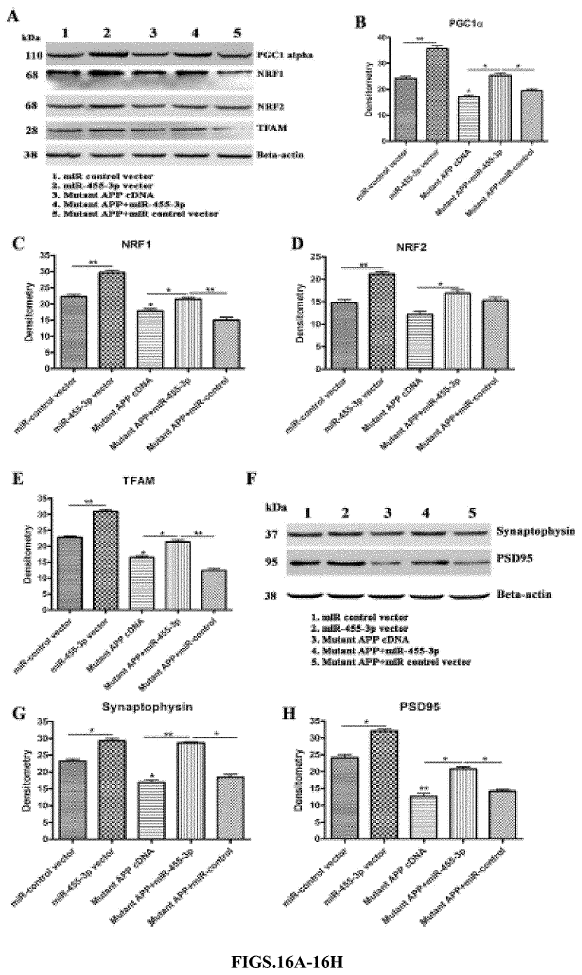

Similarly, quantitative densitometry based on immunoblot analysis revealed decreased protein levels of PGC1α, NRF1, NRF2, and TFAM by the mutant APP relative to cells transfected with miR-control. On the other hand, biogenesis protein levels were increased in miR-455-3p and mutant APP cDNA co-transfected cells relative to transfected with mutant APP alone, indicating that miR-455-3p enhances biogenesis activity (FIGS. 16A-16E).

FIGS. 16A to 16H—Immunoblotting for mitochondrial biogenesis and synaptic genes. (FIG. 16A) Representative western blot images for PGC1α, NRF1, NRF2, TFAM, and beta-actin proteins levels in 1) Neuroblastoma cells transfected with the miR-control vector, 2) Neuroblastoma cells transfected with the miR-455-3p expression vector, 3) Neuroblastoma cells transfected with the mutant APP cDNA, 4) Neuroblastoma cells co-transfected with the miR-455-3p and mutant APP cDNA, and 5) Neuroblastoma cells co-transfected with the mutant APP and miR-control vector. Quantitative measurement of the levels of (FIG. 16B) the PGC1α protein (FIG. 16C) NRF1, (FIG. 16D) NRF2, and (FIG. 16E) TFAM using densitometry in 1) Cells transfected with the miR-control vector, 2) Cells transfected with the miR-455-3p expression vector, 3) Cells transfected with mutant APP cDNA, 4) Cells co-transfected with the miR-455-3p and mutant APP cDNA, and 5) Cells co-transfected with the mutant APP and miR-control vector. (FIG. 16F) Representative western blot images for synaptophysin, PSD95, and beta-actin proteins levels in 1) Cells transfected with the miR-control vector, 2) Cells transfected with the miR-455-3p expression vector, 3) Cells transfected with the mutant APP cDNA, 4) Cells co-transfected with the miR-455-3p and mutant APP cDNA, and 5) Cells co-transfected with the mutant APP and miR-control vector. Quantitative measurement of the levels of (G) the synaptophysin protein and (FIG. 16H) PSD95 using densitometry in same groups of cells. (*P<0.05) (**P<0.01)

A synaptic protein analysis revealed the significantly reduced levels of synaptophysin and PSD95 genes at mRNA (Table 9) and protein levels (FIGS. 16F-16H) in cells transfected with mutant APP relative to controls. On the other hand, synaptic protein levels were increased significantly in miR-455-3p co-transfected cells relative to transfected with mutant APP alone.

Effect of miR-455-3p on mitochondrial dynamics (fission and fusion). Further, to confirm the effect of miR-455-3p on mitochondrial dynamics genes, the inventors studied the expression of mitochondrial fission proteins (DRP1 and FIS1) and fusion proteins (OPA1, Mfn1, and Mfn2) at mRNA and protein levels. As shown in SI Table 4, mRNA levels of DRP1 and FIS1 were significantly increased in cells transfected with mutant APP cDNA compared to controls. On the contrary, cells co-transfected with miR-455-3p and mutant APP cDNA showed reduced levels of fission genes DRP1 and FIS1 relative to mutant APP cells (Table 9). As expected, mRNA levels of mitochondrial fusion genes (OPA1, Mfn1, and Mfn2) were significantly reduce in mutant APP cells, but upregulated in the cells co-transfected with miR-455-3p and mutant APP cDNA (Table 9).

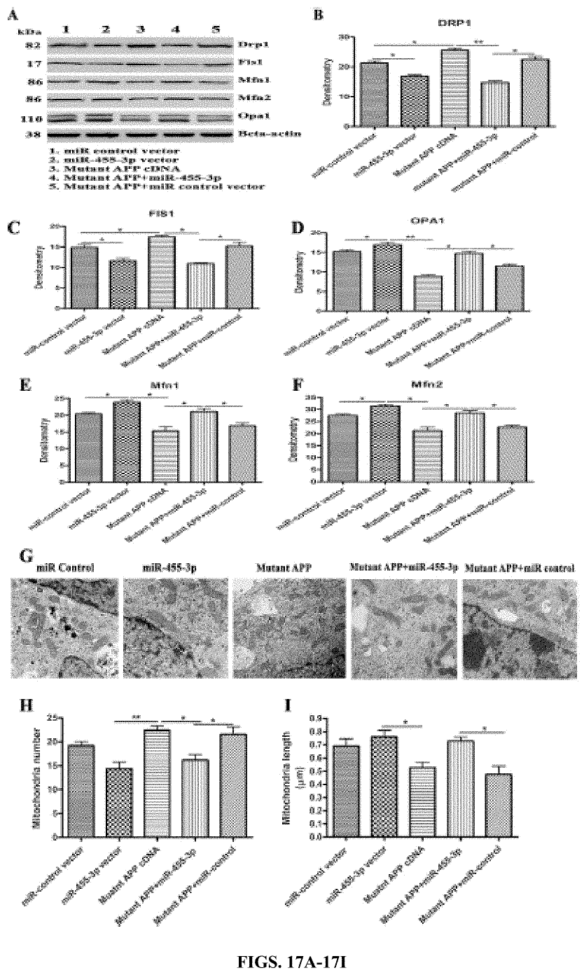

The immunoblotting data agreed with mRNA observations (FIG. 17A-17F). These results conclude that miR-455-3p reduce fission proteins and enhances fusion proteins.

FIGS. 17A to 17I—Immunoblotting for mitochondrial dynamic genes. (FIG. 17A) Representative western blot images for DRP1, FIS1, OPA1, Mfn1, Mfn2 and beta-actin proteins levels in: 1) Neuroblastoma cells transfected with the miR-control vector, 2) Neuroblastoma cells transfected with the miR-455-3p expression vector, 3) Neuroblastoma cells transfected with the mutant APP cDNA, 4) Neuroblastoma cells co-transfected with the miR-455-3p and mutant APP cDNA, and 5) Neuroblastoma cells co-transfected with the mutant APP and miR-control vector. Quantitative measurement of the levels of (FIG. 17B) the DRP1, (FIG. 17C) FIS1, (FIG. 17D) OPA1, (FIG. 17E) Mfn1 and (FIG. 17F) Mfn2 proteins using densitometry in the same groups of cells. (FIG. 17G) Representative TEM images of Neuroblastoma cells transfected with the miR-control vector, the miR-455-3p vector, mutant APP cDNA, co-transfected with mutant APP and miR-455-3p, and co-transfected with mutant APP and miR-control, showing mitochondrial organization (600 nm magnification). (FIG. 17H) Quantification of the number of mitochondria in the Neuroblastoma cells transfected with miR-control vector, the miR-455-3p vector, mutant APP cDNA, co-transfected with mutant APP and miR-455-3p, and co-transfected with mutant APP and miR-control. (FIG. 17I) Quantification of the size of mitochondria (μm) in the neuroblastoma cells transfected with miR-control vector, the miR-455-3p vector, mutant APP cDNA, co-transfected with mutant APP and miR-455-3p, and co-transfected with mutant APP and miR-control. (*P<0.05) (**P<0.01).

Effect of miR-455-3p on mitochondrial morphology. The inventors also studied mitochondrial number and length in mutant APP cells transfected with miR-455-3p in order to determine the effect of miR-455-3p on mitochondrial morphology. As shown in FIG. 17G increased numbers of mitochondria were found in cells transfected with mutant APP cDNA relative to miR-control vector transfected cells, indicating that mutant APP, fragments mitochondria. However, significantly reduced numbers of mitochondria were found in cells co-transfected with miR-455-3p and mutant APP relative to cells transfected with mutant APP alone (FIG. 17H).

Conversely, the mitochondrial length was decreased in the mutant APP cells, whereas, cells co-transfected with miR-455-3p and mutant APP cDNA showed increased in mitochondrial length compared to mutant APP cells alone (FIG. 17I). Taken together, these analyses confirmed that miR-455-3p had the potential to control and/or repair the abnormal mitochondrial dynamics caused by toxic effects of mutant APP.

This example targeted miR-455-3p as a therapeutic candidate for persons with AD. The inventors demonstrate the therapeutic use for miR-455-3p through the modulation of the APP processing. Additionally, it was found that miR-455-3p promote cell survival and activates synaptic genes, also indicating that miR-455-3p may have a protective role in AD pathogenesis through the modulation of defective mitochondrial biogenesis and mitochondrial dynamics.

The bioinformatics analysis showed that miR-455-3p targets several other genes (e.g., BAX, BMF, TGFBR3L, and HIF1 [http://targetscan.org]) that have important roles in apoptotic pathways. However, in current study, a focus was on the regulation of APP processing by miR-455-3p, and how it overcomes the toxic effect of mutant APP on cell survival, mitochondrial biogenesis, mitochondrial dynamics, and synaptic activity.

The two constitutive binding sites of miR-455-3p at the 3′UTR region of the APP gene provides better regulation and modulation of miRNA moieties. Overexpression of miR-455-3p in mutant APP cells, significantly reduced the levels of mutant full-length APP and Aβ₍₁₋₄₀₎ & Aβ₍₁₋₄₂₎. These findings strongly suggest that miR-455-3p is a potential candidate to treat AD. In this study, the inventors used a mutant APP cDNA construct that robustly expressed the human mutant APP protein in HT22 cells [32]. The inventors found that an overexpression of mutant APP, causes mitochondrial, synaptic, and autophagy/mitophagy abnormalities in hippocampal neurons, in addition to reduced cell survival, leading to neuronal dysfunction [32]. mRNA levels of APP were significantly increased (2745-fold) in mutant APP cDNA transfected cells. Despite this huge increase, miR-455-3p was capable of efficiently suppress the toxic effect of mutant APP on cell survival, mitochondrial biogenesis/dynamics, and synaptic activity. It was also found that miR-455-3p not only interact and modulate full-length APP levels, but also suppress C99 fragment of APP, which is responsible for the generation of Aβ₍₁₋₄₀₎ and Aβ₍₁₋₄₂₎ peptides [39].

The inventors also identified a supportive role of miR-455-3p in mitochondrial biogenesis, consistent with Zhang et al. (2015), who reported that miR-455 regulates brown adipogenesis via a novel HIF1an-AMPK-PGC1α signaling network [40]. The inventors confirmed that overexpression of the mutant APP cDNA caused the suppression of PGC1α and other mitochondrial biogenesis genes. In this example, over-expression of miR-455-3p not only increased PGC1α but also increased the expression of their downstream effector genes NRF-1, NRF-2, and TFAM. Therefore, upregulation of mitochondrial biogenesis events eventually improved the overall mitochondrial functions. These findings are supported by the inventors' electron microscopic analysis of neuroblastoma cells transfected with mutant APP cDNA and miR-455-3p, miR-455-3p was associated with an increase in mitochondrial length and a decrease in the number of mitochondria number. Further, analysis of mitochondrial dynamics proteins showed the suppression of fission protein (DRP1 and FIS1) and upregulation of fusion proteins (OPA1, Mfn1, and Mfn2) by miR-455-3p. Based on these observations, it was found that overexpression of miR-455-3p maintain the normal mitochondrial activity by reducing the toxic effects of Aβ on mitochondria.

Additionally, the upregulation of the key synaptic genes synaptophysin and PSD95 in presence of miR-455-3p provides new evidence that miR-455-3p has a role in the regulation of synaptic activity. The improved cell survival and extended dendrites in miR-455-3p overexpressing cells, further supports its possible role in the neuronal proliferation. This example is the first to describe that miR-455-3p has significant role progression and pathogenesis in AD.

To understand the impact of miR-455-3p in the brain, overexpressed (transgenic) and/or (depleted) knockout mouse models for miR-455-3p can be characterized. Further, transgenic and knockout lines of miR-455-3p mice with different mutant APP mice (APP—Tg2576), APP/PS1, 5×FAD and others) and study abnormal APP processing, C-terminal fragments such as C83 & C99, Aβ levels and mitochondrial biogenesis, mitochondrial dynamics and synaptic activities in double mutant mice (Tg miR455-3p×APP Tg mice; KO miR455-3p×APP Tg mice) relative to APP Tg mice alone can be studied.

In summary, miR-455-3p increases cell survival and promotes cell proliferation, and overexpression of miR-455-3p dramatically decreases full-length APP expression, the C-terminal fragment of APP and reduces Aβ₍₁₋₄₀₎ and Aβ₍₁₋₄₂₎ levels. Further, increased levels of miR-455-3p also increase mitochondrial biogenesis, mitochondrial fusion, and synaptic genes. These findings suggest the negative role of miR-455-3p in abnormal APP processing and its positive role in the regulation of mitochondrial biogenesis/dynamics and synaptic plasticity make it a valuable potential molecule worthy of research to identify AD therapy.

### Example 4

MiRNAs are present in small, subcellular compartments of the neuron such as neural dendrites, synaptic vesicles, and synaptosomes are known as synaptic miRNAs. Synaptic miRNAs involved in governing multiple synaptic functions that lead to healthy brain functioning and synaptic activity. However, the precise role of synaptic miRNAs had not previously been determined in AD progression. This example emphasizes the presence of small RNAs and miRNAs at the synapse, synaptic vesicles, and synaptosomes. The inventors summarize some potential synaptic miRNAs, their functions, and how these synaptic miRNAs regulate the levels of synaptic proteins locally at the synapse. Finally, the impact of synaptic miRNAs in AD progression concerning the synaptic ATP production, mitochondrial function, and synaptic activity is discussed.

The purpose of this example was to assess the role of functional and dysfunctional synapse-associated miRNAs in AD progression. The inventors looked into the presence of small RNAs and miRNAs at the synapse, the synaptic vesicles, and synaptosomes and accessed their roles in regulating synaptic activity in AD and other neurodegenerative diseases. In this example, the inventors provide results that has focused on synaptic miRNAs as possible therapeutic agents that capable of preventing or delaying AD onset or progression.

The presence of small RNAs populations was discovered in the synaptic vesicles (SVs). SVs are small neuronal presynaptic organelles that carry the neurotransmitters and release them to the synaptic cleft. Before, it was presumed that synaptic vesicles contain the neurotransmitters only. Li et al (2015), identified the presence of small ribonucleic acids (sRNAs), in the synaptic vesicles isolated from the electric organ of Torpedo californica in addition to the regular neurotransmitters [17]. Most of the sRNAs are transfer RNA molecules termed as tRNA fragments [17]. Not even Torpedo californica, but the SVs of the mouse brain contain abundant levels of sRNAs, including transfer RNA fragments (trfRNAs) and miRNAs [17]. These findings show that SV sRNAs are conserved and are possibly involved in transcriptional and translational regulation of local synaptic proteins. Besides, the release of neurotransmitters, now it is exciting to know whether SV contained sRNAs regulate the synaptic activity or change dendritic excitability. The SVs specific trfRNAs and miRNAs, both could directly regulate local protein synthesis and could have broad implications on neurotransmission and synaptic activity.

MiR-134 and miR-138 are the two potent neural miRNAs that played important roles in synapse development and synaptic activity. MiRNAs present throughout the cells, and some miRNAs are localized to cellular organelles. Subcellular compartmentalization and localization of miRNAs, microRNA induced silencing complex, and target mRNA have been observed to localize in multiple subcellular compartments, including rough endoplasmic reticulum, P-bodies, stress granules in the trans-Golgi network, early and late endosomes, multivesicular bodies, lysosomes, mitochondria, and the nucleus.

The synaptic vesicles extracted from mouse CNS that contains several small RNAs, transfer-RNA, and miRNAs (miR-128-1, miR-99a, miR-100, miR-22, and miR-127) included high copy numbers of miR-128-1 and miR-99a relative to other miRNAs. The most abundant miRNA, miR-128-1, is important for neuronal development, synaptogenesis, and post-mitotic neuronal functioning. The members of the miR-99 family (miR-99a) have been shown to co-enrich with polyribosomes in mammalian neurons and to regulate the mammalian target of the rapamycin (mTOR) pathway. Other than miR-128-1 and miR-99a, several other miRNAs (miR-124a-3p, miR-136-5p, and miR-376a-3p) were also abundantly expressed within synaptoneurosomes isolated from the prion-infected forebrain of animal model. These synaptoneurosomes associated or synaptosome-specific miRNAs (termed synapto-miRs) could be very important in AD research and other neurodegenerative diseases. The potential synaptic miRNAs and their main functions are summarized in Table 10.

TABLE 10
Summary of synaptic miRNAs, their location and molecular functions.

| Synaptic miRNAs | Location | Functions | Target genes | References |
|---|---|---|---|---|
| MiR-134 | Synaptodendritic | Synaptic development, synaptic | Limk1 | [59] |
| | compartments | maturation, and synaptic | | |
| | | plasticity | | |
| MiR-138 | Brain | Local protein translation at the | Lypla1 | [62] |
| | | synapse and synaptic plasticity. | | |
| MiR-146 and MiR- | Synaptic fraction | Synaptic plasticity | | [15] |
| 125a | | | | |
| MiR-125a | Synaptosome | Suppression and/or degradation | P5D95 | [16] |
| | | of target mRNAs at local | | |
| | | synapses | | |
| MiR-128-1, | Neuron (Synaptic | Regulates motor behavior by | extracellular | [17, 63] |
| | vesicles) | modulating neuronal signaling | signal-regulated | |
| | | networks and excitability. | kinase ERK2 | |
| | | | network’ | |
| MiR-99a | Polyribosomes in | Regulate the mammalian target of | | [17, 64] |
| | mammalian | the rapamycin (mTOR) pathway | | |
| | neurons | | | |
| MiR-338 | Brain | Mitochondrial activity and ATP | COXIV | [79] |
| | | production | | |
| MiR-151a-5p | | Cellular respiration and ATP | Cytochrome b | [81] |
| | | production | (Cytb) | |
| MiR-423-3p | | ATP production and energy | Cox6a2 | [82] |
| | | metabolism | | |
| MiR-125b | Brain | Synaptic deficits, neurotrophic | Synapsin-2 (SYN- | [83] |
| | | deficits, and astrogliosis | 2) and 15- | |
| | | | lipoxygenase (15- | |
| | | | LOX) | |
| MiR-124 | Brain | Synaptic dysfunction and | PTPN1 | [19] |
| | | memory loss in AD | | |
| MiR-30b | | Synaptic integrity in AD | Ephrin type-B | [20] |
| | | | receptor 2 (ephB2), | |
| | | | Sirtuin1 (sirt1), | |
| | | | and Glutamate | |
| | | | ionotropic receptor | |
| | | | AMPA type | |
| | | | subunit 2 (GluA2). | |

The role of miRNAs has been determined in the regulation of synaptic activity in AD and other neurodegenerative diseases. MiRNA plays an important role in regulating synaptic plasticity, including synaptogenesis, alteration of synaptic morphology, and modification of synaptic functions. However, the function of synapse enriched miRNAs is not determined in great detail in AD, although miRNAs, enriched at synaptic compartments, directly regulate local protein synthesis (synaptic proteins) and play a crucial role in neurotransmission. For a long time, synaptosomes were prepared from the postmortem brains of AD patients and studied for AD-associated deficits such as neurotransmission, including acetylcholine, glutamate, and c-aminobutyric acid (GABA) systems. These synaptosome studies identified decreased levels of neprilysin in AD patients. In other studies, synaptosomes were prepared from the frontal cortical region of AD postmortem brains to study Aβ aggregates in a synaptosomal fraction. Therefore, the study on the synaptosomal miRNAs was needed in AD.

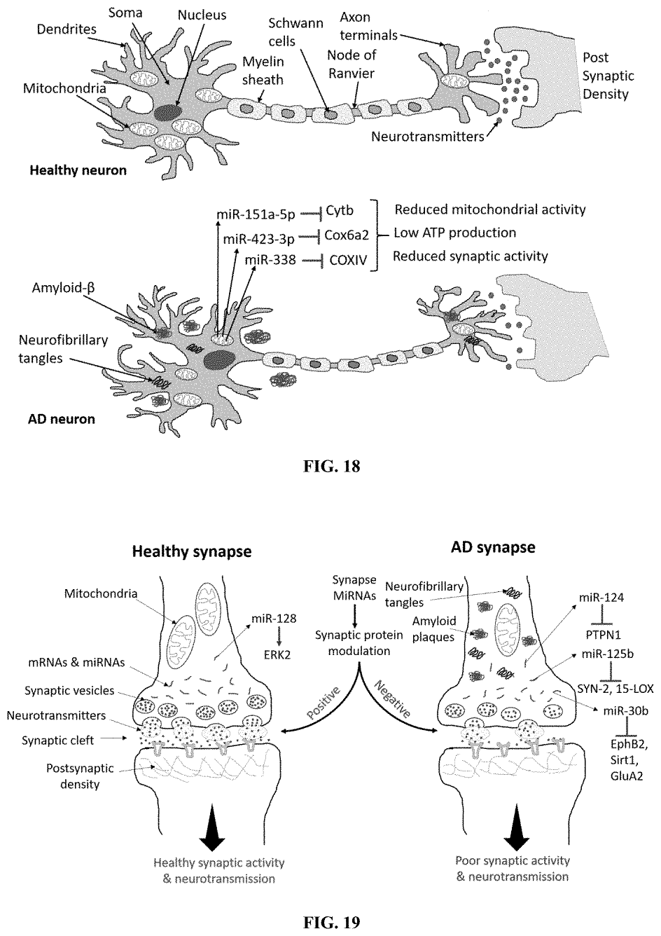

FIG. 18 shows a proposed mechanism of action mt miRNAs on the mitochondrial function, ATP production, and synaptic activity, which mechanism is not a limitation of the present invention. FIG. 18 is a pictorial representation of miRNAs action on mitochondrial function and ATP production in the neuron. Healthy neurons showed the normal synaptic activity and neurotransmission, whereas in diseases state such as AD, Aβ, and tau toxicities cause miRNAs alteration in the neuron. Increased level of miRNAs reduces their target gene expression, those are important for mitochondrial function. Reduction of mitochondrial proteins leads to impaired mitochondrial function, reduced ATP, and poor synaptic activity.

Although definitive proof that miRNAs function in a localized manner in the neurons is still lacking, it is quite possible that miRNAs localized in dendrites and axon terminals could modulate the expression of local mRNAs and proteins. As shown in FIG. 19, the positive and negative regulation of proteins at the synapse by local miRNAs could alter synaptic functions. In a healthy state, cell homeostasis maintains balanced miRNAs levels, where miRNAs function in a positive way to maintain healthy cells functioning, e.g., miR-128 regulates the ERK2 pathway [63]. Healthy neurons showed normal synaptic activity and neurotransmission. In AD state, the deregulation of synaptic miRNAs caused by the Aβ and tau toxicities. Increased level of miRNAs-miR-124, miR-125b, and miR-30b leads to the downregulation of their target gene/proteins (PTPN1, SYN-2, 15-LOX, EphB2, Sirt1, and GluA2) which are important for synaptic plasticity [19,20,83]. Suppression of synaptic and other proteins could impair synapse activity and neurotransmission (FIG. 19).

FIG. 19 is a working model of synaptic miRNAs: miRNAs localized in dendrites and axon terminals could modulate the expression of local mRNAs and proteins. Positive and negative regulation of synaptic proteins by local miRNAs could alter synaptic function. Neurons not affected by AD (healthy neuron) showed balanced miRNAs levels and normal synaptic activity and synaptic neurotransmission. Brain-specific miR-128 showed the positive modulation of synaptic activity via the ERK2 pathway. On the other hand in AD state, the deregulation of the brain-specific miRNAs (miR-124 and miR-125b) and other miR-30b negatively modulate the synaptic activity via suppression of their target genes. The suppression of synaptic and other proteins leads to impaired synaptic activity and neurotransmission.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES—EXAMPLE 1

1. Lukiw J (2013) Circular RNA (circRNA) in Alzheimer's Disease (AD). Frontiers in Genetics 4:307.
2. Reddy P H, et al. (2017) A critical evaluation of neuroprotective and neurodegenerative MicroRNAs in Alzheimer's disease. Biochem Biophys Res Commun 483 (4): 1156-1165.
3. LaFerla F M, Green K N, Oddo S (2007) Intracellular amyloid-β in Alzheimer's disease. Nat Rev Neuro 8(7):449-509.
4. Mattson MP (2004) Pathways towards and away from Alzheimer's disease. Nature 430(7000):631-639.
5. Reddy P H, et al. (2010) Amyloid-β and mitochondria in aging and Alzheimer's disease: Implications for synaptic damage and cognitive decline. J Alz Dis 20(2010):S499-S512.
6. Kumar P, et al. (2013) Circulating miRNA biomarkers for Alzheimer's disease. Plos One 8(7):e69807.
7. Zafari S, et al. (2015) Circulating biomarkers panels in Alzheimer's disease. Gerontology 61(6):497-503.
8. Zhao Y, et al. (2015) microRNA-based biomarkers and the diagnosis of Alzheimer's disease. Frontiers in Neurology 6:162.
9. Bartel D P (2007) MicroRNAs: Genomics, Biogenesis, Mechanism and Function. Cell 116(2):281-297.
10. Adlakha Y K, Saini N (2014) Brain microRNAs and insights into biological functions and therapeutic potential of brain enriched miRNA-128. Mol Cancer 13:33.
11. Kumar S, Reddy P H (2016) Are circulating microRNAs peripheral biomarkers for Alzheimer's disease? Biochim Biophys Acta 1862(9):1617-1627.
12. Ha M, Kim V N (2015) Regulation of microRNA biogenesis. Nat Rev Mol Cell Biol 15(8):509-524.
13. Boon R A, Vickers K C (2007) Intercellular Transport of MicroRNAs. Arterioscler Thromb Vasc Biol 33(2)186-192.
14. Schipper H M, et al. (2007) microRNA expression in Alzheimer blood mononuclear cells. Gene Regul Syst Biol 20(1):263-274.
15. Alexandrov P N, et al. (2012) microRNA (miRNA) speciation in Alzheimer's disease (AD) cerebrospinal fluid (CSF) and extracellular fluid (ECF). Int J Biochem Mol Biol 3(4)365-373.
16. Geekiyanage H, et al. (2012) Blood serum miRNA: Non-invasive biomarkers for Alzheimer's disease. Exp Neurol 235(2):491-496.
17. Cheng L, et al. (2015) Prognostic serum miRNA biomarker associated with Alzheimer's disease shows concordance with neuropsychological and neuroimaging assessment. Mol Psychiatry 20 (10):1188-1196.
18. Galimberti D, et al. (2014) Circulating miRNAs as potential biomarkers in Alzheimer's disease. J Alz Dis 42(4):1261-1267.
19. Tan L, et al. (2014) Circulating miR-125 as a biomarker of Alzheimer's disease. J Neurol Sci 336(1-2):52-56.
20. Satoh J, Kino Y, Niida S (2015) MicroRNA-Seq data analysis pipeline to identify blood biomarkers for Alzheimer's disease from public data. Biomarker Insights 10:21-31.
21. Dong H, et al. (2015) Serum microRNA profiles serve as novel biomarkers for the diagnosis of Alzheimer's disease. Dis. Markers (2015):625659.
22. Williams J, et al. (2016) Are microRNAs true sensors of ageing and cellular senescence?. Ageing Res Rev 51568-1637(16):30168-30164.
23. Wu H Z Y, et al. (2016) Circulating microRNAs as biomarkers of Alzheimer's disease: A systemic review. J Alz Dis 49(3):755-766.
24. Liu C C, et al. (2013) Apolipoprotein E and Alzheimer disease: risk, mechanisms and therapy. Nat Rev Neurol 9(2):106-118.
25. Zheng J, et al. (2016) MicroRNA-455-3p Inhibits Tumor Cell Proliferation and Induces Apoptosis in HCT116 Human Colon Cancer Cells. Med Sci Monit 22:4431-4437.
26. Zhang Z, et al. (2015) MiR-455-3p regulates early chondrogenic differentiation via inhibiting Runx2. FEBS Lett 589(23):3671-3678.
27. Chen W, et al. (2016) MicroRNA-455-3p modulates cartilage development and degeneration through modification of histone H3 acetylation. Biochim Biophys Acta 1863(12):2881-2891.
28. Lalevée S, Lapaire O, Büler M (2014) miR455 is linked to hypoxia signaling and is deregulated in preeclampsia. Cell Death Dis 5:e1408.
29. Zhang H, et al. (2015) MicroRNA-455 regulates brown adipogenesis via a novel HIF1an-AMPK-PGC1α signaling network. EMBO Rep 16(10):1378-1393.
30. Freischmidt A, et al. (2014) Serum microRNAs in patients with genetic amyotrophic lateral sclerosis and pre-manifest mutation carriers. Brain 137(Pt 11):2938-2950.
31. Wang N, et al. (2015) Profiling and initial validation of urinary microRNAs as biomarkers in IgA nephropathy. PeerJ 3:e990.
32. Yan S, et al. (2017) Altered microRNA profiles in plasma exosomes from mesial temporal lobe epilepsy with hippocampal sclerosis. Oncotarget 8(3):4136-4146.
33. Lugli G, et al. (2015) Plasma Exosomal miRNAs in Persons with and without Alzheimer Disease: Altered Expression and Prospects for Biomarkers. PLoS One10(10):e0139233.
34. Knyazev E N, et al. (2016) MicroRNA hsa-miR-4674 in Hemolysis-Free Blood Plasma Is Associated with Distant Metastases of Prostatic Cancer. Bull Exp Biol Med 161(1):112-115.
35. Hsiao K, et al. (1996) Correlative memory deficits, Abeta elevation, and amyloid plaques in transgenic mice. Science 274(5284):99-102.
36. Manczak M, et al. (2016) Protective effects of reduced dynamin-related protein 1 against amyloid beta-induced mitochondrial dysfunction and synaptic damage in Alzheimer's disease. Hum Mol Genet 25(22):4881-4897.
37. Swingler T E, et al. (2012) The expression and function of microRNAs in chondrogenesis and osteoarthritis. Arthritis Rheum 64(6):1909-1919.
38. Das P, Golde T (2006) Dysfunction of TGF-beta signaling in Alzheimer's disease. J Clin Invest 116(11):2855-2857.
39. von Bernhardi R, et al. (2015) Role of TGFβ signaling in the pathogenesis of Alzheimer's disease. Front Cell Neurosci 9:426.
40. Ferrer I, Blanco R (2000) N-myc and c-myc expression in Alzheimer disease, Huntington disease and Parkinson disease. Brain Res Mol Brain Res. 77(2):270-276.
41. Rosenmann H, et al. (2004) An association study of a polymorphism in the heparin sulfate proteoglycan gene (perlecan, HSPG2) and Alzheimer's disease. Am J Med Genet B Neuropsychiatr Genet 128B(1):123-125.
42. Lee H G, et al. (2006) Ectopic expression of phospho-Smad2 in Alzheimer's disease: uncoupling of the transforming growth factor-beta pathway?. J Neurosci Res 84(8):1856-1861.
43. Lee H G, et al. (2009) The neuronal expression of MYC causes a neurodegenerative phenotype in a novel transgenic mouse. Am J Pathol 174(3):891-897.
44. Cheng J S, et al. (2009) Collagen VI protects neurons against Abeta toxicity. Nat Neurosci 12(2):119-121.
45. Donovan L E, et al. (2013) Exploring the potential of the platelet membrane proteome as a source of peripheral biomarkers for Alzheimer's disease. Alzheimers Res Ther 5(3):32.
46. Xie K, et al. (2013) Tenascin-C deficiency ameliorates Alzheimer's disease-related pathology in mice. Neurobiol Aging 34(10):2389-2398.
47. Mastroeni D, et al. (2013) Reduced RAN expression and disrupted transport between cytoplasm and nucleus; a key event in Alzheimer's disease pathophysiology. PLoS One 8(1):e53349.
48. Godfrey A C, et al. (2013) Serum microRNA expression as an early marker for breast cancer risk in prospectively collected samples from the Sister Study cohort. Breast Cancer Res 15(3):R42.
49. Shi W L, et al. (2016) Integrated miRNA and mRNA expression profiling in fetal hippocampus with Downsyndrome. J Biomed Sci 23(1):48.
50. Kumar S, et al. (2014) Severity of hepatitis C virus (genotype-3) infection positively correlates with circulating microRNA-122 in patients sera. Dis Markers (2014):435476.
51. Livak K J, Schmittgen T D (2001) Analysis of relative gene expression data using real-time quantitative PCR and the 2−ΔΔCT method. Methods 25(4):402-408.
52. Alhasan A H, et al. (2016) Circulating microRNA signature for the diagnosis of very high-risk prostate cancer. Proc Natl Acad Sci USA 113(38):10655-10660.

### REFERENCES—EXAMPLE 2

Bertoni-Freddari, C., Fattoretti, P., Casoli, T., Caselli, U., and Meier-Ruge, W. (1996). Deterioration threshold of synaptic morphology in aging and senile dementia of Alzheimer's type. Anal. Quant. Cytol. Histol. 18, 209-213.
Burton, T., Liang, B., Dibrov, A., and Amara, F. (2002). Transforming growth factor-beta-induced transcription of the Alzheimer beta-amyloid precursor protein gene involves interaction between the CTCFcomplex and Smads. Biochem. Biophys. Res. Commun. 295, 713-723. doi: 10.1016/S0006-291X(02)00725-8
Chen, W., Chen, L., Zhang, Z., Meng, F., Huang, G., Sheng, P., et al. (2016). MicroRNA-455-3p modulates cartilage development and degeneration through modification of histone H3 acetylation. Biochim. Biophys. Acta 1863, 2881-2891. doi: 10.1016/j.bbamcr.2016.09.010
Cheng, C. M., Shiah, S. G., Huang, C. C., Hsiao, J. R., and Chang, J. Y. (2016). Upregulation of miR-455-5p by the TGF-b-SMAD signalling axis promotes the proliferation of oral squamous cancer cells by targeting UBE2B. J. Pathol. 240, 38-49. doi: 10.1002/path.4752
DeKosky, S. T., Scheff, S. W., and Styrene, S. D. (1996). Structural correlates of cognition in dementia: quantification and assessment of synapse change. Neurodegeneration 5, 417-421. doi: 10.1006/neur.1996.0056
De Rossi, P., Buggia-Prévot, V., Clayton, B. L., Vasquez, J. B., van Sanford, C., Andrew, R. J., et al. (2016). Predominant expression of Alzheimer's disease associated BIN1 in mature oligodendrocytes and localization to white matter tracts. Mol. Neurodegener. 11:59. doi: 10.1186/s13024-016-0124-1
Du, H., Guo, L., Yan, S., Sosunov, A. A., McKhann, G. M., and Yan, S. S. (2010). Early deficits in synaptic mitochondria in an Alzheimer's disease mouse model. Proc. Natl. Acad. Sci. U.S.A. 107, 18670-18675. doi: 10.1073/pnas.1006586107
Ferguson, S. A., Panos, J. J., Sloper, D., and Varma, V. (2017). Neurodegenerative markers are increased in postmortemBA21 tissue from African Americans with Alzheimer's disease. J. Alzheimers Dis. 59, 57-66. doi: 10.3233/JAD-170204
Hamam, R., Ali, A. M., Alsaleh, K. A., Kassem, M., Alfayez, M., Aldahmash, A., et al. (2016). microRNA expression profiling on individual breast cancer patients identifies novel panel of circulating microRNA for early detection. Sci. Rep. 6:25997. doi: 10.1038/srep25997
Jung, E. S., Choi, H., Song, H., Hwang, Y. J., Kim, A., Ryu, H., et al. (2016). p53-dependent SIRT6 expression protects Ab42-induced DNA damage. Sci. Rep. 6:25628. doi:10.1038/srep25628
Jung, E. S., Hong, H., Kim, C., and Mook-Jung, I. (2015). Acute ER stress regulates amyloid precursor protein processing through ubiquitin-dependent degradation. Sci. Rep. 5:8805. doi: 10.1038/srep08805
Khan, T. K., and Alkon, D. L. (2016). An internally controlled peripheral biomarker for Alzheimer's disease: Erk1 and Erk2 responses to the inflammatory signal bradykinin. Proc. Natl. Acad. Sci. U.S.A. 103, 13203-13207. doi: 10.1073/pnas.0605411103
Kumar, S., Chawla, Y. K., Ghosh, S., and Chakraborti, A. (2014). Severity of hepatitis C virus (genotype-3) infection positively correlates with circulating microRNA-122 in patients sera. Dis. Markers 2014:435476. doi: 10.1155/2014/435476
Kumar, S., and Reddy, P. H. (2016). Are circulating microRNAs peripheral biomarkers for Alzheimer's disease?. Biochim. Biophys. Acta 1862, 1617-1627. doi: 10.1016/j.bbadis.2016.06.001
Kumar, S., Vijayan, M., and Reddy, P. H. (2017). MicroRNA-455-3p as a potential peripheral biomarker for Alzheimer's disease. Hum. Mol. Genet. 26, 3808-3822. doi: 10.1093/hmg/ddx267
Kuruva, C. S., Manczak, M., Yin, X., Ogunmokun, G., Reddy, A. P., and Reddy, P. H. (2017). Aqua-soluble DDQ reduces the levels of Drp1 and Ab and inhibits abnormal interactions between Ab and Drp1 and protects Alzheimer's disease neurons from Ab- and Drp1-induced mitochondrial and synaptic toxicities. Hum. Mol. Genet. 26, 3375-3395. doi: 10.1093/hmg/ddx226
LaFerla, F. M., Green, K. N., and Oddo, S. (2007). Intracellular amyloid-beta in Alzheimer's disease. Nat. Rev. Neurosci. 8, 499-509. doi: 10.1038/nrn2168
Li, Q., Athan, E. S., Wei, M., Yuan, E., Rice, S. L., Vonsattel, J. P., et al. (2004). TP73 allelic expression in human brain and allele frequencies in Alzheimer's disease. BMC Med. Genet. 5:14. doi: 10.1186/1471-2350-5-14
Li, Y. J., Ping, C., Tang, J., and Zhang, W. (2016). MicroRNA-455 suppresses non-small cell lung cancer through targeting ZEB1. Cell Biol. Int. 40, 621-628. doi: 10.1002/cbin.10584
Liu, J., Zhang, J., Li, Y., Wang, L., Sui, B., and Dai, D. (2016). MiR-455-5p acts as a novel tumor suppressor in gastric cancer by down-regulating RAB18. Gene 592, 308-315. doi: 10.1016/j.gene.2016.07.034
Malkki, H. (2015). Alzheimer disease: NGF gene therapy activates neurons in the AD patient brain. Nat. Rev. Neurol. 11:548. doi: 10.1038/nrneuro1.2015.170
Mattson, M. P. (2004). Pathways towards and away from Alzheimer's disease. Nature 430, 631-639. doi: 10.1038/nature02621
McGeer, P. L., and McGeer, E. G. (1995). The inflammatory response system of brain: implications for therapy of Alzheimer and other neurodegenerative diseases. Brain Res. Rev. 21, 195-218. doi: 10.1016/0165-0173(95)00011-9
Nunomura, A., Perry, G., Aliev, G., Hirai, K., Takeda, A., Balraj, E. K., et al. (2001). Oxidative damage is the earliest event in Alzheimer disease. J. Neuropathol. Exp. Neurol. 60, 759-767. doi: 10.1093/jnen/60.8.759
Qin, L., Zhang, Y., Lin, J., Shentu, Y., and Xie, X. (2016). MicroRNA-455 regulates migration and invasion of human hepatocellular carcinoma by targeting Runx2. Oncol. Rep. 36, 3325-3332. doi: 10.3892/or.2016.5139
Reddy, P. H. (2006). Amyloid precursor protein-mediated free radicals and oxidative damage: implications for the development and progression of Alzheimer's disease. J. Neurochem. 96, 1-13. doi: 10.1111/j.1471-4159.2005.03530.x
Reddy, P. H., and Beal, M. F. (2008). Amyloid beta, mitochondrial dysfunction and synaptic damage: implications for cognitive decline in aging and Alzheimer's disease. Trends Mol. Med. 14, 45-53. doi: 10.1016/j.molmed.2007.12.002
Reddy, P. H., Manczak, M., Mao, P., Calkins, M. J., Reddy, A. P., and Shirendeb, U. (2010). Amyloid-beta and mitochondria in aging and Alzheimer's disease: implications for synaptic damage and cognitive decline. J. Alzheimers Dis. 20, S499-S512. doi: 10.3233/JAD-2010-100504
Reddy, P. H., Tonk, S., Kumar, S., Vijayan, M., Kandimalla, R., Kuruva, C. S., et al. (2017). A critical evaluation of neuroprotective and neurodegenerative MicroRNAs in Alzheimer's disease. Biochem. Biophys. Res. Commun. 483, 1156-1165. doi: 10.1016/j.bbrc.2016.08.067
Reddy, P. H., Tripathi, R., Troung, Q., Tirumala, K., Reddy, T. P., Anekonda, V., et al. (2012). Abnormal mitochondrial dynamics and synaptic degeneration as early events in Alzheimer's disease: implications to mitochondria targeted antioxidant therapeutics. Biochim. Biophys. Acta 1822, 639-649. doi: 10.1016/j.bbadis.2011.10.011
Shackleton, B., Crawford, F., and Bachmeier, C. (2017). Apolipoprotein Emediated modulation of ADAM10 in Alzheimer's disease. Curr. Alzheimer Res. 14, 578-585. doi: 10.2174/1567205014666170203093219
Sindi, I. A., Tannenberg, R. K., and Dodd, P. R. (2014). Role for the neurexinneuroligin complex in Alzheimer's disease. Neurobiol. Aging 35, 746-756. doi: 10.1016/j.neurobiolaging.2013.09.032
Slifer, M. A., Martin, E. R., Bronson, P. G., Browning-Large, C., Doraiswamy, P. M., Welsh-Bohmer, K. A., et al. (2006). Lack of association between UBQLN1 and Alzheimer disease. Am. J. Med. Genet. B Neuropsychiatr. Genet. 141B, 208-213. doi: 10.1002/ajmg.b.30298
Swerdlow, R. H. (2011). Brain aging, Alzheimer's disease, and mitochondria. Biochim. Biophys. Acta 1812, 1630-1639. doi: 10.1016/j.bbadis.2011. 08.012
Tampellini, D., and Gouras, G. K. (2010). Synapses, synaptic activity and intraneuronal abeta in Alzheimer's disease. Front. Aging Neurosci. 2:13. doi: 10.3389/fnagi.2010.00013
Terry, R. D., Masliah, E., Salmon, D. P., Butters, N., DeTeresa, R., Hill, R., et al. (1991). Physical basis of cognitive alterations in Alzheimer's disease: synapse loss is the major correlate of cognitive impairment. Ann. Neurol. 30, 572-580. doi: 10.1002/ana.410300410
Tóth, M. E., Szegedi, V., Varga, E., Juhász, G., Horvath, J., Borbély, E., et al. (2013). Overexpression of Hsp27 ameliorates symptoms of Alzheimer's disease in APP/PS1 mice. Cell Stress Chaperones 18, 759-771. doi: 10.1007/s12192-013-0428-9
Vallortigara, J., Whitfield, D., Quelch, W., Alghamdi, A., Howlett, D., Hortobágyi, T., et al. (2016). Decreased levels of VAMP2 and monomeric alphasynuclein correlate with duration of dementia. J. Alzheimers Dis. 50, 101-110. doi: 10.3233/JAD-150707
Wilcock, D. M., Hurban, J., Helman, A. M., Sudduth, T. L., McCarty, K. L., Beckett, T. L., et al. (2015). Down syndrome individuals with Alzheimer's disease have a distinct neuroinflammatory phenotype compared to sporadic Alzheimer's disease. Neurobiol. Aging 36, 2468-2474. doi: 10.1016/j.neurobiolaging.2015.05.016
Williams, J., Smith, F., Kumar, S., Vijayan, M., and Reddy, P. H. (2016). Are microRNAs true sensors of ageing and cellular senescence?. Ageing Res. Rev. 35, 350-363. doi:10.1016/j.arr.2016.11.008
World Alzheimer Report (2015). Publication Alzheimer's Association. Zhao, Y., Yan, M., Yun, Y., Zhang, J., Zhang, R., Li, Y., et al. (2017). MicroRNA-455-3p functions as a tumor suppressor by targeting eIF4E in prostate cancer. Oncol. Rep. 37, 2449-2458. doi: 10.3892/or.2017.5502
Zheng, J., Lin, Z., Zhang, L., and Chen, H. (2016). MicroRNA-455-3p inhibits tumor cell proliferation and induces apoptosis in HCT116 human colon cancer cells. Med. Sci. Monit. 18, 4431-4437. doi: 10.12659/MSM.898452
Zhu, X., Perry, G., Smith, M. A., and Wang, X. (2013). Abnormal mitochondrial dynamics in the pathogenesis of Alzheimer's disease. J. Alzheimers Dis. 33, S253-S262. doi: 10.3233/JAD-2012-129005

### REFERENCES—EXAMPLE 3

1. Selkoe. DJ, Alzheimer's disease: genes, proteins, and therapy, Physiol. Rev. 81 (2001) 741-766.
2. Mattson. MP, Pathways towards and away from Alzheimer's disease, Nature. 430 (2004) 631-639.
3. LaFerla. F M, Green. KN, Oddo. S, Intracellular amyloid-beta in Alzheimer's disease, Nat. Rev. Neurosci. 8 (2007) 499-509.
4. Reddy. PH, Manczak. M, Mao. P, Calkins. MJ, Reddy. AP, Shirendeb. U, Amyloid-beta and mitochondria in aging and Alzheimer's disease: implications for synaptic damage and cognitive decline, J. Alzheimers Dis. 20 (2010) S499-S512.
5. Mao. P, Reddy. PH, Aging and amyloid beta-induced oxidative DNA damage and mitochondrial dysfunction in Alzheimer's disease: implications for early intervention and therapeutics, Biochim. Biophys. Acta. 1812 (2011) 1359-1370.
6. Terry. RD, Masliah. E, Salmon. DP, Butters. N, DeTeresa. R, Hill. R, Hansen. L A, Katzman. R, Physical basis of cognitive alterations in Alzheimer's disease: synapse loss is the major correlate of cognitive impairment, Ann. Neurol. 30 (1991) 572-580.
7. DeKosky. ST, Scheff. SW, Styrene. S D, Structural correlates of cognition in dementia: quantification and assessment of synapse change, Neurodegeneration. 5 (1996) 417-421.
8. Nunomura. A, Perry. G, Aliev. G, Hirai. K, Takeda. A, Balraj. EK, Jones. PK, Ghanbari. H, Wataya. T, Shimohama. S, Chiba. S, Atwood. CS, Petersen. RB, Smith. M A, Oxidative damage is the earliest event in Alzheimer disease, J. Neuropathol. Exp. Neurol. 60 (2001) 759-767.
9. McGeer. P L, McGeer. EG, Inflammation, autotoxicity, and Alzheimer disease, Neurobiol. Aging. 22 (2001) 799-809.
10. Reddy. PH, Amyloid precursor protein-mediated free radicals and oxidative damage: implications for the development and progression of Alzheimer's disease, J. Neurochem. 96 (2006) 1-13.
11. Reddy. PH, Beal. MF, Amyloid beta, mitochondrial dysfunction and synaptic damage: implications for cognitive decline in aging and Alzheimer's disease, Trends Mol. Med. 14 (2008) 45-53.
12. Manczak. M, Mao. P, Nakamura. K, Bebbington. C, Park. B, Reddy. PH, Neutralization of granulocyte macrophage colony-stimulating factor decreases amyloid beta 1-42 and suppresses microglial activity in a transgenic mouse model of Alzheimer's disease, Hum. Mol. Genet. 18 (2009) 3876-3893.
13. Du. H, Guo. L, Yan. S, Sosunov. A A, McKhann, G M, Yan, S S, Early deficits in synaptic mitochondria in an Alzheimer's disease mouse model, Proc. Natl. Acad. Sci. USA. 107 (2010) 18670-18675.
14. Swerdlow. RH, Brain aging, Alzheimer's disease, and mitochondria, Biochim. Biophys. Acta. 1812 (2011) 1630-1639.
15. Reddy. PH, Tripathi. R, Troung. Q, Tirumala. K, Reddy. TP, Anekonda. V, Shirendeb. UP, Calkins. MJ, Reddy. AP, Mao. P, Manczak. M, Abnormal mitochondrial dynamics and synaptic degeneration as early events in Alzheimer's disease: implications to mitochondria-targeted antioxidant therapeutics, Biochim. Biophys. Acta. 1822 (2012) 639-649.
16. Zhu. X, Perry. G, Smith. M A, Wang. X, Abnormal mitochondrial dynamics in the pathogenesis of Alzheimer's disease, J. Alzheimers Dis. 33 (2013) S253-S262.
17. Reddy. PH, Tonk. S, Kumar. S, Vijayan. M, Kandimalla. R, Kuruva. CS, Reddy. AP, A critical evaluation of neuroprotective and neurodegenerative MicroRNAs in Alzheimer's disease, Biochem. Biophys. Res. Commun. 483 (2017) 1156-1165.
18. Kumar. S, Chawla. YK, Ghosh. S, Chakraborti. A, Severity of hepatitis C virus (genotype-3) infection positively correlates with circulating microRNA-122 in patients sera, Dis. Markers. (2014) 435476.
19. Kumar. S, Reddy. PH, Are circulating microRNAs peripheral biomarkers for Alzheimer's disease?, Biochim. Biophys. Acta. 1862 (2016) 1617-1627.
20. Vijayan. M, Reddy. PH, Peripheral biomarkers of stroke: Focus on circulatory microRNAs, Biochim. Biophys. Acta. 1862 (2016) 1984-1993.
21. Kumar. S, Vijayan. M, Reddy. PH, MicroRNA-455-3p as a potential peripheral biomarker for Alzheimer's disease, Hum. Mol. Genet. 26 (2017) 3808-3822.
22. Williams. J, Smith. F, Kumar. S, Vijayan. M, Reddy. PH, Are microRNAs true sensors of aging and cellular senescence?, Ageing Res. Rev. 35 (2017) 350-363.
23. Reddy. PH, Williams. J, Smith. F, Bhatti. J S, Kumar. S, Vijayan. M, Kandimalla. R, Kuruva. CS, Wang. R, Manczak. M, Yin. X, Reddy. AP, MicroRNAs, Aging, Cellular Senescence, and Alzheimer's Disease, Prog. Mol. Biol. Transl. Sci. 2017; 146:127-71.
24. Vijayan. M, Kumar. S, Yin. X, Zafer. D, Chanana. V, Cengiz. P, Reddy. PH, Identification of novel circulatory microRNA signatures linked to patients with ischemic stroke, Hum. Mol. Genet. 27 (2018) 2318-2329.
25. Zhao. J, Yue. D, Zhou. Y, Jia. L, Wang. H, Guo. M, Xu. H, Chen. C, Zhang. J, Xu. L, The Role of MicroRNAs in Aβ Deposition and Tau Phosphorylation in Alzheimer's Disease, Front. Neurol. 8 (2017) 342.
26. Vilardo. E, Barbato. C, Ciotti. M, Cogoni. C, Ruberti. F, MicroRNA-101 regulates amyloid precursor protein expression in hippocampal neurons, J. Biol. Chem. 285 (2010) 18344-18351.
27. Long. J M, Lahiri. DK, MicroRNA-101 downregulates Alzheimer's amyloid-β precursor protein levels in human cell cultures and is differentially expressed, Biochem. Biophys. Res. Commun. 404 (2011) 889-895.
28. Kim. J, Yoon. H, Horie. T, Burchett. J M, Restivo. J L, Rotllan. N, Ramirez. CM, Verghese. PB, Ihara. M, Hoe. HS, Esau. C, Fernandez-Hernando. C, Holtzman. DM, Cirrito. JR, Ono. K, Kim. J, microRNA-33 Regulates ApoE Lipidation and Amyloid-β Metabolism in the Brain, J. Neurosci. 35 (2015) 14717-14726.
29. Ma. X, Liu. L, Meng. J, MicroRNA-125b promotes neurons cell apoptosis and Tau phosphorylation in Alzheimer's disease, Neurosci. Lett. 661 (2017) 57-62.
30. Long. J M, Maloney. B, Rogers. JT, Lahiri. DK, Novel upregulation of amyloid-β precursor protein (APP) by microRNA-346 via targeting of APP mRNA 5′-untranslated region: Implications in Alzheimer's disease, Mol. Psychiatry. 3 (2019) 345-363.
31. Kumar. S, Reddy. PH, MicroRNA-455-3p as a Potential Biomarker for Alzheimer's Disease: An Update, Front. Aging Neurosci. 10 (2018) 41.
32. Reddy. PH, Yin. X, Manczak. M, Kumar. S, Pradeepkiran. JA, Vijayan. M, Reddy. AP, Mutant APP and amyloid beta-induced defective autophagy, mitophagy, mitochondrial structural and functional changes and synaptic damage in hippocampal neurons from Alzheimer's disease, Hum. Mol. Genet. 27 (2018) 2502-2516.
33. Reddy. PH, Manczak. M, Yin. X, Grady. MC, Mitchell. A, Kandimalla. R, Reddy. AP, Protective effects of a natural product, curcumin, against amyloid β induced mitochondrial and synaptic toxicities in Alzheimer's disease, J. Investig. Med. 64 (2016) 1220-1234.
34. Manczak. M, Kandimalla. R, Fry. D, Sesaki. H, Reddy. PH, Protective effects of reduced dynamin-related protein 1 against amyloid beta-induced mitochondrial dysfunction and synaptic damage in Alzheimer's disease, Hum. Mol. Genet. 25 (2016) 5148-5166.
35. Gupta. P, Bhattacharjee. S, Sharma. A R, Sharma. G, Lee. SS, Chakraborty. C, miRNAs in Alzheimer Disease—A Therapeutic Perspective, Curr. Alzheimer Res. 14 (2017) 1198-1206.
36. Zhang. S, Wu. W, Jiao. G, Li. C, Liu. H, MiR-455-3p activates Nrf2/ARE signaling via HDAC2 and protects osteoblasts from oxidative stress, Int. J. Biol. Macromol. 107 (2018) 2094-2101.
37. Zhan. T, Huang. X, Tian. X, Chen. X, Ding. Y, Luo. H, Zhang. Y, Downregulation of MicroRNA-455-3p Links to Proliferation and Drug Resistance of Pancreatic Cancer Cells via Targeting TAZ, Mol. Ther. Nucleic Acids. 10 (2018) 215-226.
38. Li. Z, Meng. Q, Pan. A, Wu. X, Cui. J, Wang. Y, Li. L, MicroRNA-455-3p promotes invasion and migration in triple negative breast cancer by targeting tumor suppressor EI24, Oncotarget. 8 (2017) 19455-19466.
39. Multhaup. G, Huber. O, Buée. L, Galas. MC, Amyloid Precursor Protein (APP) Metabolites APP Intracellular Fragment (AICD), Aβ42, and Tau in Nuclear Roles, J. Biol. Chem. 290 (2015) 23515-23522.
40. Zhang. H, Guan. M, Townsend. KL, Huang. TL, An. D, Yan. X, Xue. R, Schulz. TJ, Winnay. J, Mori. M, Hirshman. MF, Kristiansen. K, Tsang. J S, White. AP, Cypess. A M, Goodyear. LJ, Tseng. YH, MicroRNA-455 regulates brown adipogenesis via a novel HIF1an AMPK-PGC1α signaling network, EMBO Rep. 16 (2015) 1378-1393.

What is claimed is:

1. A method of protecting neuronal cells from Aβ-induced toxicities in a subject comprising:
providing the subject in need of treatment for an Aβ-induced toxicity with an agent that increases the miRNA-445-3p in the subject, wherein an increase in miR-455-3p expression in the neuronal cells enhances neuronal cell survival.

2. The method of claim 1, wherein the agent that increases the miRNA-445-3p in the subject is a nucleic acid vector that expresses miRNA-445-3p in neuronal cells of the subject.

3. The method of claim 1, wherein the agent enhances at least one of: mitochondrial biogenesis, enhances synaptic activity or mitochondrial health.

4. The method of claim 1, further comprising providing the subject with a cyclooxygenase inhibitor, a Catecholamine transferase inhibitor, a protein kinases inhibitor, a Neurotransmitter transporter inhibitor, a Renin-angiotensin system inhibitor, a EGFR tyrosine kinase inhibitor, or a HMG-CoA reductase inhibitor.

5. The method of claim 1, further comprising the step of identifying a subject in need of treatment for the Aβ-induced toxicity by detecting the presence or the increase in miRNA-445-3p to produce a score that is indicative of a likelihood of developing AD, wherein a higher score relative to a healthy control indicates that the patient is likely to have the prognosis for transitioning to classified AD, wherein the healthy control is derived from a non-AD patient with no clinical evidence of AD.

6. The method of claim 5, wherein the patient is identified at least 0.1, 0.9, 2.0, 3.5, or greater than 3.5 years prior to reaching clinical disease classification.

7. The method of claim 5, wherein the step of assessing comprises RT-PCR, qRT-PCR, biochip, singleplexed or multiplexed RT-PCR.

8. The method of claim 5, further comprising assessing at least one additional biomarker selected from: hsa-miR-3613-3p and hsa-miR-4668-5p, which is up-regulated.

9. The method of claim 5, further comprising assessing at least one additional biomarker selected from: hsa-mir-320d-2, hsa-miR-378h, hsa-miR-3921, hsa-miR-6805-5p, hsa-miR-92a-3p, hsa-miR-3613-5p, which is down-regulated.

10. The method of claim 5, wherein obtaining the dataset associated with the sample comprises obtaining the sample and processing the sample to experimentally determine the dataset, or wherein obtaining the dataset associated with the sample comprises receiving the dataset from a third party that has processed the sample to experimentally determine the dataset.

11. The method of claim 5, further comprising identifying a relative of the patient at risk for AD by obtaining a score from a dataset associated with a blood, serum, or plasma sample from a relative of the AD patient prior to reaching clinical disease classification.

12. The method of claim 5, wherein the healthy control is a pre-determined average level derived from a healthy individual with no clinically documented evidence of AD.

13. The method of claim 5, wherein at least one of:
the miR-455-3p has a greater that 20-fold increase in expression in Braak stage V and VI compared to controls;
the expression of miR-3613-3p is higher in the brain tissues at Braak stage V when compared to controls;
the expression of miR-4668-5p up-regulated in AD brains at Braak stages IV, V, and VI, but less than miR-455-3p or MiR-4674;
the expression of mir-6722 was down-regulated in AD serum samples Braak stages I to III, but increased in the AD patients at Braak stage VI, V, and VI;
the upregulation of miR-455-3p was significantly higher in postmortem brains from AD patients at Braak stage V having an ApoE (3/4) genotype when compared to controls; or
the Braak stage can be differentiated between Stage I to III versus Brask Stage IV to VI by comparing the expression of miR-455-3p, miR-3613-3p, miR-4668-5p, and mir-6722.

14. A method for treating at least one of amyloid precursor protein (APP) processing, amyloid beta (Aβ) formation, defective mitochondrial biogenesis/dynamics and synaptic damage in AD progression in a subject suspected of having Alzheimer's disease (AD) comprising:
providing the subject in need of treatment for an Aβ-induced toxicity with an agent that increases the miRNA-445-3p in the subject, wherein an increase in miR-455-3p expression in the neuronal cells enhances neuronal cell survival, wherein the amount is sufficient to at least one of correct amyloid precursor protein (APP) processing, prevent amyloid beta (Aβ) formation, decrease defective mitochondrial biogenesis/dynamics or reduce or prevent synaptic damage in AD progression.

15. The method of claim 14, wherein the expression of miRNA-445-3p in neuronal cells reduces a level of mutant APP, Aβ(1-40) and Aβ(1-42), and C99 by miR-455-3p.

16. The method of claim 14, wherein the agent that increases the expression of miRNA-455-3p in a subject is a nucleic acid vector that expresses miRNA-445-3p in neuronal cells of the subject, wherein an increase in miR-455-3p expression in the neuronal cells enhances neuronal cell survival, wherein the amount is sufficient to at least one of correct amyloid precursor protein (APP) processing, prevent amyloid beta (Aβ) formation, decrease defective mitochondrial biogenesis/dynamics or reduce or prevent synaptic damage in AD progression.

17. The method of claim 14, further comprising administering a cyclooxygenase inhibitor, a Catecholamine transferase inhibitor, a protein kinases inhibitor, a Neurotransmitter transporter inhibitor, a Renin-angiotensin system inhibitor, a EGFR tyrosine kinase inhibitor, or a HMG-CoA reductase inhibitor.

18. The method of claim 14, further comprising assessing at least one additional biomarker selected from: hsa-miR-3613-3p and hsa-miR-4668-5p, which is up-regulated.

19. The method of claim 14, further comprising assessing at least one additional biomarker selected from: hsa-mir-320d-2, hsa-miR-378h, hsa-miR-3921, hsa-miR-6805-5p, hsa-miR-92a-3p, hsa-miR-3613-5p, which is down-regulated.

20. The method of claim 14, wherein the patient is identified at least 0.1, 0.9, 2.0, 3.5, or greater than 3.5 years prior to reaching clinical disease classification.

21. The method of claim 14, wherein the step of assessing comprises RT-PCR, qRT-PCR, biochip, singleplexed or multiplexed RT-PCR.

22. A method for treating a subject with Alzheimer's Disease (AD) comprising:
obtaining a blood, serum, cerebrospinal fluid, or plasma sample from the AD patient;
assessing the dataset for a presence or an increase in an amount of miRNA-445-3p; and
if the subject has a decrease in miRNA-445-3p expression or is in need of an increase in the expression of miRNA-445-3p, providing the subject in need of treatment for an Aβ-induced toxicity an agent that increases the miRNA-445-3p in the subject, wherein an increase in miR-455-3p expression in the neuronal cells enhances neuronal cell survival.

23. The method of claim 22, further comprising administering a cyclooxygenase inhibitor, a Catecholamine transferase inhibitor, a protein kinases inhibitor, a Neurotransmitter transporter inhibitor, a Renin-angiotensin system inhibitor, a EGFR tyrosine kinase inhibitor, or a HMG-CoA reductase inhibitor.

24. The method of claim 22, wherein the agent that increases the miRNA-445-3p in the subject is a nucleic acid vector that expresses miRNA-445-3p in neuronal cells of the subject.

25. The method of claim 22, wherein the step of assessing comprises RT-PCR, qRT-PCR, biochip, singleplexed or multiplexed RT-PCR.

26. The method of claim 22, further comprising assessing at least one additional biomarker selected from: hsa-miR-3613-3p and hsa-miR-4668-5p, which is up-regulated.

27. The method of claim 22, further comprising assessing at least one additional biomarker selected from: hsa-mir-320d-2, hsa-miR-378h, hsa-miR-3921, hsa-miR-6805-5p, hsa-miR-92a-3p, hsa-miR-3613-5p, which is down-regulated.

28. The method of claim 22, wherein obtaining the dataset associated with the sample comprises obtaining the sample and processing the sample to experimentally determine the dataset, or wherein obtaining the dataset associated with the sample comprises receiving the dataset from a third party that has processed the sample to experimentally determine the dataset.

29. The method of claim 22, further comprising identifying a relative of the patient at risk for AD by obtaining a score from a dataset associated with a blood, serum, or plasma sample from a relative of the AD patient prior to reaching clinical disease classification.

30. The method of claim 22, wherein the healthy control is a pre-determined average level derived from a healthy individual with no clinically documented evidence of AD.

31. A composition comprising a recombinant miRNA-445-3p nucleic acid sufficient to upregulate the expression of miRNA-445-3p.

32. A method of treating a subject in need of therapy for Alzheimer's Disease comprising:
identifying a subject suspected of having Alzheimer's Disease; and
providing the subject with an effective amount of an agent that upregulates the expression of miRNA-445-3p in the subject in need of therapy for Alzheimer's Disease.

33. The method of claim 32, wherein the recombinant miRNA-455-3p nucleic acid is a nucleic acid vector that expresses miRNA-445-3p in neuronal cells of a subject.

34. The method of claim 32, wherein the agent is an expression vector that expresses miRNA-455-3p.

35. The method of claim 32, wherein the agent is a recombinant or synthetic miRNA that comprises miRNA-445-3p.

36. The method of claim 32, wherein the agent is a miRNA-445-3p miRNA in a liposome, vesicle, polymer, or nanoparticles.


SEQUENCE LISTING (10 sequences)

SEQ ID NO: 1 - DNA, 21 bp, Artificial Sequence
  Synthetic primer
gcagtccatgggcatatacac

SEQ ID NO: 2 - DNA, 21 bp, Artificial Sequence
  Synthetic primer
gcagtccacgggcatatacac

SEQ ID NO: 3 - DNA, 21 bp, Artificial Sequence
  Synthetic primer
ctgggctcgggacgcgcggct

SEQ ID NO: 4 - DNA, 23 bp, Artificial Sequence
  Synthetic primer
agggaaaaaaaaaaggatttgtc

SEQ ID NO: 5 - DNA, 24 bp, Artificial Sequence
  Synthetic primer
acaaaaaaaaaagcccaacccttc

SEQ ID NO: 6 - DNA, 22 bp, Artificial Sequence
  Synthetic primer
ggcctcaggcaggcgcacccga

SEQ ID NO: 7 - DNA, 22 bp, Artificial Sequence
  Synthetic primer
gggtgggccaggctgtggggcg

SEQ ID NO: 8 - DNA, 23 bp, Artificial Sequence
  Synthetic primer
cgcttcggcagcacatatactaa

SEQ ID NO: 9 - DNA, 23 bp, Artificial Sequence
  Synthetic primer
tatggaacgcttcacgaatttgc

SEQ ID NO: 10 - DNA, 22 bp, Artificial Sequence
  Synthetic primer
agtacttttgaacccttttcca